SEQUENCE LISTING (130 sequences)

SEQ ID NO: 1 - protein, 6 aa, synthetic construct
SIFSSA

SEQ ID NO: 2 - protein, 6 aa, synthetic construct
IGSSDT

SEQ ID NO: 3 - protein, 9 aa, synthetic construct
KYGLGGFVY

SEQ ID NO: 4 - protein, 118 aa, synthetic construct
QVQLQESGGGLVQAGGSLRLSCATSGSIFSSAVMGWYRQAPGNERELVALIGSSDTTDYSNSVKGRFTISRDNAKNTAYLRMNSLKPEDTAVYYCTAVKYGLGGFVYWGQGTQVTVSS

SEQ ID NO: 5 - DNA, 18 bp, synthetic construct
  source 1..18: mol_type = other DNA
agcatcttcagtagcgct

SEQ ID NO: 6 - DNA, 18 bp, synthetic construct
  source 1..18: mol_type = other DNA
attggcagtagcgatacc

SEQ ID NO: 7 - DNA, 27 bp, synthetic construct
  source 1..27: mol_type = other DNA
aagtacgggctggggggatttgtctac

SEQ ID NO: 8 - DNA, 354 bp, synthetic construct
  source 1..354: mol_type = other DNA
caggtgcagctgcaggagtctgggggaggcttggtgcaggctggggggtctctgagactctcctgtgcaacctctggaagcatcttcagtagcgctgtcatgggctggtaccgccaggctccagggaatgagcgcgagttggtcgcactcattggcagtagcgataccacagactattcaaactctgtgaagggccgattcaccatctccagagacaacgccaagaacacggcctatctgcgcatgaatagcctgaaacctgaggacacagccgtctattactgtactgccgtcaagtacgggctggggggatttgtctactggggccaggggacccaggtcaccgtctcctca

SEQ ID NO: 9 - protein, 6 aa, synthetic construct
LTFDSY

SEQ ID NO: 10 - protein, 7 aa, synthetic construct
SIWSGDS

SEQ ID NO: 11 - protein, 17 aa, synthetic construct
ASFLHSANYHMRAKWGY

SEQ ID NO: 12 - protein, 127 aa, synthetic construct
QVQLQESGGGLVQPEGSLRLSCAASGLTFDSYAIGWFRQAPGKEREFVAASIWSGDSGHYTGSVKGRFTISRDNAKNTVDLQMNSLKPEDTAVYYCAASASFLHSANYHMRAKWGYWGQGTQVTVSS

SEQ ID NO: 13 - DNA, 18 bp, synthetic construct
  source 1..18: mol_type = other DNA
ctcaccttcgatagctat

SEQ ID NO: 14 - DNA, 21 bp, synthetic construct
  source 1..21: mol_type = other DNA
agtatctggagtggtgatagc

SEQ ID NO: 15 - DNA, 51 bp, synthetic construct
  source 1..51: mol_type = other DNA
gcgtctttcttgcacagtgctaattaccacatgcgggcaaaatggggttac

SEQ ID NO: 16 - DNA, 381 bp, synthetic construct
  source 1..381: mol_type = other DNA
caggtgcagctgcaggagtctgggggaggattggtgcagcctgagggctctctgagactctcctgtgcagcctctggactcaccttcgatagctatgccatcggctggttccgccaggctccagggaaggagcgtgagtttgtagcagcaagtatctggagtggtgatagcggacactatacaggttccgtgaaaggccgattcaccatctcccgagacaacgccaagaacacggtggatctgcaaatgaacagcctgaaacccgaggacacggccgtttattactgtgcagccagcgcgtctttcttgcacagtgctaattaccacatgcgggcaaaatggggttactggggccaggggacccaggtcaccgtctcctca

SEQ ID NO: 17 - protein, 6 aa, synthetic construct
RTFSTY

SEQ ID NO: 18 - protein, 6 aa, synthetic construct
RWSGGT

SEQ ID NO: 19 - protein, 14 aa, synthetic construct
RGSGSYSPTYRWDY

SEQ ID NO: 20 - protein, 123 aa, synthetic construct
QVQLQESGGGLVQTGDSLRLSCAVSGRTFSTYSVGWFRQAPGKEREFVALRWSGGTTYYADSVVGRFTVSRDNAKNTVYLEMNSLKPEDTAVYYCAADRGSGSYSPTYRWDYWGQGTQVTVSS

SEQ ID NO: 21 - DNA, 18 bp, synthetic construct
  source 1..18: mol_type = other DNA
cgcaccttcagtacctat

SEQ ID NO: 22 - DNA, 18 bp, synthetic construct
  source 1..18: mol_type = other DNA
aggtggagtggtggtacc

SEQ ID NO: 23 - DNA, 42 bp, synthetic construct
  source 1..42: mol_type = other DNA
cggggtagtggtagttactccccgacatatcgctgggactat

SEQ ID NO: 24 - DNA, 369 bp, synthetic construct
  source 1..369: mol_type = other DNA
caggtgcagctgcaggagtctgggggagggttggtgcaaactggggactctctgagactctcctgtgcagtgtctggacgcaccttcagtacctattccgtgggctggttccgccaggctccaggaaaggagcgtgagtttgtagcgcttaggtggagtggtggtaccacatactatgcagactccgtggtgggccggttcaccgtctccagagacaatgccaagaacacggtgtatctggaaatgaacagcctgaaacctgaggacacggccgtttattactgtgcagcagatcggggtagtggtagttactccccgacatatcgctgggactattggggccaggggacccaggtcaccgtctcctca

SEQ ID NO: 25 - protein, 6 aa, synthetic construct
LAFSMY

SEQ ID NO: 26 - protein, 7 aa, synthetic construct
IISSGDS

SEQ ID NO: 27 - protein, 15 aa, synthetic construct
KFRYYFSTSPGDFDS

SEQ ID NO: 28 - protein, 125 aa, synthetic construct
QVQLQESGGGLVQPGGSLRLSCAASGLAFSMYTMGWFRQAPGKEREFVAMIISSGDSTDYADSVKGRFTISRDNGKNTVYLQMDSLKPEDTAVYYCAAPKFRYYFSTSPGDFDSWGQGTQVTVSS

SEQ ID NO: 29 - DNA, 18 bp, synthetic construct
  source 1..18: mol_type = other DNA
ctcgcctttagtatgtat

SEQ ID NO: 30 - DNA, 18 bp, synthetic construct
  source 1..18: mol_type = other DNA
attatttcaagtggtgat

SEQ ID NO: 31 - DNA, 45 bp, synthetic construct
  source 1..45: mol_type = other DNA
aagtttcgttactactttagcacctctccaggtgattttgattcc

SEQ ID NO: 32 - DNA, 375 bp, synthetic construct
  source 1..375: mol_type = other DNA
caggtgcagctgcaggagtctggaggaggattggtacagcctgggggctctctgagactctcctgtgcagcctctggactcgcctttagtatgtataccatgggctggttccgccaggctccagggaaggagcgtgagtttgtagcaatgattatttcaagtggtgatagcaccgactacgcagactccgtgaagggccgattcaccatctccagggacaacggcaagaacacggtgtatctgcaaatggacagcctgaaacctgaggacacggccgtttattactgtgcagccccaaagtttcgttactactttagcacctctccaggtgattttgattcctggggccaggggacccaggtcaccgtctcctca

SEQ ID NO: 33 - protein, 6 aa, synthetic construct
SILSIN

SEQ ID NO: 34 - protein, 6 aa, synthetic construct
ITSGGS

SEQ ID NO: 35 - protein, 7 aa, synthetic construct
TGWGPLD

SEQ ID NO: 36 - protein, 116 aa, synthetic construct
QVQLQESGGGLVQVGGSLRLSCAASGSILSINAMGWYRQAPGKQRELVAAITSGGSTNYADSVKGRFTISRDNAKNMLYLQMNSLKPEDTAVYYCHVVTGWGPLDWGQGTQVTVSS

SEQ ID NO: 37 - DNA, 18 bp, synthetic construct
  source 1..18: mol_type = other DNA
agcatcctcagtatcaat

SEQ ID NO: 38 - DNA, 18 bp, synthetic construct
  source 1..18: mol_type = other DNA
attactagtggtggtagc

SEQ ID NO: 39 - DNA, 21 bp, synthetic construct
  source 1..21: mol_type = other DNA
acgggttggggtcccctagac

SEQ ID NO: 40 - DNA, 348 bp, synthetic construct
  source 1..348: mol_type = other DNA
caggtgcagctgcaggagtctgggggaggcttggtgcaggttggggggtctctacgactctcctgtgcagcctctggaagcatcctcagtatcaatgccatgggctggtaccgccaggctccagggaagcagcgcgagttggtcgcagctattactagtggtggtagcacaaactatgcagactccgtgaagggccgattcaccatctccagagacaacgccaagaacatgctatatctgcaaatgaacagcctgaaacctgaggacacagccgtctattactgccacgtcgttacgggttggggtcccctagactggggccaggggacccaggtcaccgtctcctca

SEQ ID NO: 41 - protein, 6 aa, synthetic construct
FTLSSN

SEQ ID NO: 42 - protein, 6 aa, synthetic construct
ITSGLS

SEQ ID NO: 43 - protein, 9 aa, synthetic construct
RGWGPPRDY

SEQ ID NO: 44 - protein, 118 aa, synthetic construct
QVQLQESGGGLVQAGGSLRLSCAASGFTLSSNDMGWYRQAPGKQRELVATITSGLSTNYADSVKGRFTISRDNAKNTVFLQMNSLKIEDTAVYYCEVERGWGPPRDYWGHGTQVTVSS

SEQ ID NO: 45 - DNA, 18 bp, synthetic construct
  source 1..18: mol_type = other DNA
tttaccttaagtagcaat

SEQ ID NO: 46 - DNA, 18 bp, synthetic construct
  source 1..18: mol_type = other DNA
attactagtggtctgagc

SEQ ID NO: 47 - DNA, 27 bp, synthetic construct
  source 1..27: mol_type = other DNA
aggggttggggaccgccgagggactac

SEQ ID NO: 48 - DNA, 354 bp, synthetic construct
  source 1..354: mol_type = other DNA
caggtgcagctgcaggagtctgggggaggcttggtgcaagctggggggtctctgagactctcctgcgcagcctctggatttaccttaagtagcaatgacatgggctggtaccgccaggctccagggaagcagcgcgaattggtcgcaactattactagtggtctgagcacaaactatgcagactccgtgaagggccgattcaccatctccagagacaacgccaagaacacggtgtttctgcaaatgaatagcctgaaaattgaggacacagccgtctattactgtgaggtagagaggggttggggaccgccgagggactactggggccacgggacccaggtcaccgtctcctca

SEQ ID NO: 49 - protein, 6 aa, synthetic construct
RAFSSY

SEQ ID NO: 50 - protein, 7 aa, synthetic construct
IHWTGAA

SEQ ID NO: 51 - protein, 13 aa, synthetic construct
PSGSYWPPKRYDY

SEQ ID NO: 52 - protein, 123 aa, synthetic construct
QVQLQESGGGLVQAGGSLRLSCAASGRAFSSYVMGWFRQAPGKEREFVAAIHWTGAATVYVDSVKGRFAISRHNAKNTVYLEMNSLKPEDTAVYYCAADPSGSYWPPKRYDYWGQGTQVTVSS

SEQ ID NO: 53 - DNA, 18 bp, synthetic construct
  source 1..18: mol_type = other DNA
cgcgccttcagtagttat

SEQ ID NO: 54 - DNA, 21 bp, synthetic construct
  source 1..21: mol_type = other DNA
attcattggactggtgctgct

SEQ ID NO: 55 - DNA, 39 bp, synthetic construct
  source 1..39: mol_type = other DNA
cctagcggtagttactggccccccaagaggtatgactac

SEQ ID NO: 56 - DNA, 369 bp, synthetic construct
  source 1..369: mol_type = other DNA
caggtgcagctgcaggagtctggaggaggattggtgcaggctgggggctctctgagactctcctgtgcagcctctggacgcgccttcagtagttatgttatgggctggtttcgccaggctccaggaaaggagcgtgagtttgtagcagctattcattggactggtgctgctacagtctatgtagactccgtgaagggtcgattcgccatctccagacacaacgccaagaacacggtgtatctggaaatgaacagcctgaaacctgaggacacggccgtttattactgtgcagcagaccctagcggtagttactggccccccaagaggtatgactactggggccaggggacccaggtcaccgtctcctca

SEQ ID NO: 57 - protein, 6 aa, synthetic construct
RTFSSV

SEQ ID NO: 58 - protein, 8 aa, synthetic construct
ISGSTGSV

SEQ ID NO: 59 - protein, 13 aa, synthetic construct
FTGTFNYQGLYDY

SEQ ID NO: 60 - protein, 124 aa, synthetic construct
QVQLQESGGGLVRAGDSLRLSCAVSGRTFSSVAMGWFRQAPGKEREFVAFISGSTGSVTYYADSVKGRFAISRDNAKNTVYLQMNSLKPEDTAVYDCAAKFTGTFNYQGLYDYWGQGTQVTVSS

SEQ ID NO: 61 - DNA, 18 bp, synthetic construct
  source 1..18: mol_type = other DNA
cgcaccttcagtagcgtt

SEQ ID NO: 62 - DNA, 24 bp, synthetic construct
  source 1..24: mol_type = other DNA
attagcgggagtactggtagtgtt

SEQ ID NO: 63 - DNA, 39 bp, synthetic construct
  source 1..39: mol_type = other DNA
tttactggtactttcaactaccaaggtctatatgactac

SEQ ID NO: 64 - DNA, 372 bp, synthetic construct
  source 1..372: mol_type = other DNA
caggtgcagctgcaggagtctgggggaggattggtccgcgctggggactctctgagactctcctgtgcagtctctggacgcaccttcagtagcgttgccatgggctggttccgccaggctccagggaaggagcgtgaatttgtagcgtttattagcgggagtactggtagtgttacatactatgcagactccgtgaagggccgattcgccatctccagagacaacgccaagaacacggtgtatctacaaatgaacagcctgaagcctgaggacacggccgtttatgactgtgcagctaagtttactggtactttcaactaccaaggtctatatgactactggggccaggggacccaggtcaccgtctcctca

SEQ ID NO: 65 - protein, 6 aa, synthetic construct
RTFSRY

SEQ ID NO: 66 - protein, 7 aa, synthetic construct
INWSGTS

SEQ ID NO: 67 - protein, 16 aa, synthetic construct
LVRNYRLGWGDGVYDH

SEQ ID NO: 68 - protein, 126 aa, synthetic construct
QVQLQESGGGLVQAGGSLRLSCAASGRTFSRYAMGWFRQAPGKERESVAAINWSGTSIFYANSVEGRFTISRDNAKNTVYLQLNSLKPEDTAVYYCAADLVRNYRLGWGDGVYDHYGQGTQVTVSS

SEQ ID NO: 69 - DNA, 18 bp, synthetic construct
  source 1..18: mol_type = other DNA
cgcaccttcagtaggtat

SEQ ID NO: 70 - DNA, 21 bp, synthetic construct
  source 1..21: mol_type = other DNA
attaattggagtggtactagc

SEQ ID NO: 71 - DNA, 48 bp, synthetic construct
  source 1..48: mol_type = other DNA
ttggttcggaactacagattgggctggggagatggcgtctatgaccac

SEQ ID NO: 72 - DNA, 378 bp, synthetic construct
  source 1..378: mol_type = other DNA
caggtgcagctgcaggagtctgggggaggattggtgcaggctgggggctctctgagactctcctgtgcagcctctggacgcaccttcagtaggtatgctatgggctggttccgccaggctccagggaaggagcgtgagtctgtagcagctattaattggagtggtactagcatattctatgcaaactccgtggagggccgattcaccatctccagagacaacgccaagaacacggtgtatctgcaactgaacagcctgaaacctgaggacacggccgtttattactgtgcagcagacttggttcggaactacagattgggctggggagatggcgtctatgaccactacggccaggggacccaggtcaccgtctcctca

SEQ ID NO: 73 - protein, 6 aa, synthetic construct
RTFSRY

SEQ ID NO: 74 - protein, 7 aa, synthetic construct
INWSGTS

SEQ ID NO: 75 - protein, 6 aa, synthetic construct
LNGMPY

SEQ ID NO: 76 - protein, 116 aa, synthetic construct
QVQLQESGGGLVQAGGSLRLSCAASGRTFSRYAMGWFRQAPGKERESVAAINWSGTSIFYANSVEGRFTISRDNAKNTVYLQLNSLKPEDTAVYYCGVVLNGMPYWGRGTQVTVSS

SEQ ID NO: 77 - DNA, 18 bp, synthetic construct
  source 1..18: mol_type = other DNA
cgcaccttcagtaggtat

SEQ ID NO: 78 - DNA, 21 bp, synthetic construct
  source 1..21: mol_type = other DNA
attaattggagtggtactagc

SEQ ID NO: 79 - DNA, 18 bp, synthetic construct
  source 1..18: mol_type = other DNA
ctgaacggcatgccctac

SEQ ID NO: 80 - DNA, 348 bp, synthetic construct
  source 1..348: mol_type = other DNA
caggtgcagctgcaggagtctgggggagggctggtgcaggctgggggctctctgagactctcctgtgcagcctctggacgcaccttcagtaggtatgctatgggctggttccgccaggctccagggaaggagcgtgagtctgtagcagctattaattggagtggtactagcatattctatgcaaactccgtggagggccgattcaccatctccagagacaacgccaagaacacggtgtatctgcaactgaacagcctgaaacctgaggacacggccgtttattactgtggagttgtcctgaacggcatgccctactggggcagagggacccaggtcaccgtctcctca

SEQ ID NO: 81 - protein, 6 aa, synthetic construct
SIFSIN

SEQ ID NO: 82 - protein, 6 aa, synthetic construct
ITSGGS

SEQ ID NO: 83 - protein, 9 aa, synthetic construct
EGWGPPVGY

SEQ ID NO: 84 - protein, 118 aa, synthetic construct
QVQLQESGGGLVQAGGSLRLSCAASGSIFSINTMGWYRQAPGKQRELVAAITSGGSTNYADSVKGRFTISRDNAKNTVHLQMNSLKPEDTAVYYCNVVEGWGPPVGYWGQGTQVTVSS

SEQ ID NO: 85 - DNA, 18 bp, synthetic construct
  source 1..18: mol_type = other DNA
agcatcttcagtatcaat

SEQ ID NO: 86 - DNA, 18 bp, synthetic construct
  source 1..18: mol_type = other DNA
attactagtggtggtagc

SEQ ID NO: 87 - DNA, 27 bp, synthetic construct
  source 1..27: mol_type = other DNA
gagggttggggaccacctgtaggctac

SEQ ID NO: 88 - DNA, 354 bp, synthetic construct
  source 1..354: mol_type = other DNA
caggtgcagctgcaggagtctgggggaggcttggtgcaggctggggggtctctgagactctcctgtgcagcctctggaagcatcttcagtatcaataccatgggctggtaccgccaggctccagggaagcagcgcgagttggtcgcagctattactagtggtggtagcacaaactatgcagactccgtgaagggccgattcaccatctccagagacaacgccaaaaacacggtacatctgcaaatgaacagcctgaaacctgaggacacagccgtctattactgtaatgtagttgagggttggggaccacctgtaggctactggggccaggggacccaggtcaccgtctcctca

SEQ ID NO: 89 - protein, 6 aa, synthetic construct
IMFGIN

SEQ ID NO: 90 - protein, 5 aa, synthetic construct
ITSSG

SEQ ID NO: 91 - protein, 9 aa, synthetic construct
AGWGPPPGY

SEQ ID NO: 92 - protein, 118 aa, synthetic construct
QVQLQESGGGLVQAGGSLRVSCEVSGIMFGINTMGWYRQAPGKQRELVAHITSSGSTNYADSVKGRFTISRDNAKKTVYLQMNSLKPEDTAVYYCNVVAGWGPPPGYWGQGTQVTVSS

SEQ ID NO: 93 - DNA, 18 bp, synthetic construct
  source 1..18: mol_type = other DNA
atcatgtttggaattaat

SEQ ID NO: 94 - DNA, 18 bp, synthetic construct
  source 1..18: mol_type = other DNA
atcactagtagtggtagc

SEQ ID NO: 95 - DNA, 27 bp, synthetic construct
  source 1..27: mol_type = other DNA
gcaggttggggtccgccacccggctac

SEQ ID NO: 96 - DNA, 354 bp, synthetic construct
  source 1..354: mol_type = other DNA
caggtgcagctgcaggagtctggaggaggcttggtgcaggctggggggtctctgagagtctcctgtgaagtctctggaatcatgtttggaattaataccatgggctggtaccgccaggctccagggaagcagcgcgaattggtcgcacacatcactagtagtggtagcacaaattatgcagactccgtgaagggccgattcaccatctccagagacaacgccaagaaaacggtgtatctgcaaatgaacagtctgaaacctgaggacacagccgtgtactactgcaatgtagttgcaggttggggtccgccacccggctactggggccaggggacccaggtcaccgtctcctca

SEQ ID NO: 97 - DNA, 375 bp, synthetic construct
  source 1..375: mol_type = other DNA
catatgaacaacgaactgagtccggtggcactgcgtcagatgagtagtgccgccggcaccacccagaccgccagtacagatgataatgccctggcctattataataccaccaaaggtggtcgctttgttctggcactgctgagcgatctgcaggatctgaaatgggcacgttttccgaaaagcgatggtaccggcaccatctataccgaactggaaccgccgagtcgttttgtgaccgataccccgaaaggtccgaaagtgaaatatctgtattttattaagggtctgaacaacctgaatcgtggtatggtgctgggtagtctggcagcaaccgttcgcctgcagtaactcgagcaccaccaccaccaccactga

SEQ ID NO: 98 - protein, 19 aa, synthetic construct
AAAYPYDVPDYGSHHHHHH

SEQ ID NO: 99 - DNA, 20 bp, synthetic construct
  source 1..20: mol_type = other DNA
ttacaaacattggccgcaaa

SEQ ID NO: 100 - DNA, 18 bp, synthetic construct
  source 1..18: mol_type = other DNA
gcgcgacattccgaagaa

SEQ ID NO: 101 - DNA, 23 bp, synthetic construct
  source 1..23: mol_type = other DNA
acaatttgcccccagcgcttcag

SEQ ID NO: 102 - DNA, 19 bp, synthetic construct
  source 1..19: mol_type = other DNA
agatttggacctgcgagcg

SEQ ID NO: 103 - DNA, 20 bp, synthetic construct
  source 1..20: mol_type = other DNA
gagcggctgtctccacaagt

SEQ ID NO: 104 - DNA, 23 bp, synthetic construct
  source 1..23: mol_type = other DNA
ttctgacctgaaggctctgcgcg

SEQ ID NO: 105 - protein, 137 aa, synthetic construct
QVQLQESGGGLVQAGGSLRLSCATSGSIFSSAVMGWYRQAPGNERELVALIGSSDTTDYSNSVKGRFTISRDNAKNTAYLRMNSLKPEDTAVYYCTAVKYGLGGFVYWGQGTQVTVSSAAAYPYDVPDYGSHHHHHH

SEQ ID NO: 106 - DNA, 411 bp, synthetic construct
  source 1..411: mol_type = other DNA
caggtgcagctgcaggagtctgggggaggcttggtgcaggctggggggtctctgagactctcctgtgcaacctctggaagcatcttcagtagcgctgtcatgggctggtaccgccaggctccagggaatgagcgcgagttggtcgcactcattggcagtagcgataccacagactattcaaactctgtgaagggccgattcaccatctccagagacaacgccaagaacacggcctatctgcgcatgaatagcctgaaacctgaggacacagccgtctattactgtactgccgtcaagtacgggctggggggatttgtctactggggccaggggacccaggtcaccgtctcctcagcggccgcatacccgtacgacgttccggactacggttcccaccaccatcaccatcac

SEQ ID NO: 107 - protein, 146 aa, synthetic construct
QVQLQESGGGLVQPEGSLRLSCAASGLTFDSYAIGWFRQAPGKEREFVAASIWSGDSGHYTGSVKGRFTISRDNAKNTVDLQMNSLKPEDTAVYYCAASASFLHSANYHMRAKWGYWGQGTQVTVSSAAAYPYDVPDYGSHHHHHH

SEQ ID NO: 108 - DNA, 438 bp, synthetic construct
  source 1..438: mol_type = other DNA
caggtgcagctgcaggagtctgggggaggattggtgcagcctgagggctctctgagactctcctgtgcagcctctggactcaccttcgatagctatgccatcggctggttccgccaggctccagggaaggagcgtgagtttgtagcagcaagtatctggagtggtgatagcggacactatacaggttccgtgaaaggccgattcaccatctcccgagacaacgccaagaacacggtggatctgcaaatgaacagcctgaaacccgaggacacggccgtttattactgtgcagccagcgcgtctttcttgcacagtgctaattaccacatgcgggcaaaatggggttactggggccaggggacccaggtcaccgtctcctcagcggccgcatacccgtacgacgttccggactacggttcccaccaccatcaccatcac

SEQ ID NO: 109 - protein, 142 aa, synthetic construct
QVQLQESGGGLVQTGDSLRLSCAVSGRTFSTYSVGWFRQAPGKEREFVALRWSGGTTYYADSVVGRFTVSRDNAKNTVYLEMNSLKPEDTAVYYCAADRGSGSYSPTYRWDYWGQGTQVTVSSAAAYPYDVPDYGSHHHHHH

SEQ ID NO: 110 - DNA, 426 bp, synthetic construct
  source 1..426: mol_type = other DNA
caggtgcagctgcaggagtctgggggagggttggtgcaaactggggactctctgagactctcctgtgcagtgtctggacgcaccttcagtacctattccgtgggctggttccgccaggctccaggaaaggagcgtgagtttgtagcgcttaggtggagtggtggtaccacatactatgcagactccgtggtgggccggttcaccgtctccagagacaatgccaagaacacggtgtatctggaaatgaacagcctgaaacctgaggacacggccgtttattactgtgcagcagatcggggtagtggtagttactccccgacatatcgctgggactattggggccaggggacccaggtcaccgtctcctcagcggccgcatacccgtacgacgttccggactacggttcccaccaccatcaccatcac

SEQ ID NO: 111 - protein, 144 aa, synthetic construct
QVQLQESGGGLVQPGGSLRLSCAASGLAFSMYTMGWFRQAPGKEREFVAMIISSGDSTDYADSVKGRFTISRDNGKNTVYLQMDSLKPEDTAVYYCAAPKFRYYFSTSPGDFDSWGQGTQVTVSSAAAYPYDVPDYGSHHHHHH

SEQ ID NO: 112 - DNA, 432 bp, synthetic construct
  source 1..432: mol_type = other DNA
caggtgcagctgcaggagtctggaggaggattggtacagcctgggggctctctgagactctcctgtgcagcctctggactcgcctttagtatgtataccatgggctggttccgccaggctccagggaaggagcgtgagtttgtagcaatgattatttcaagtggtgatagcaccgactacgcagactccgtgaagggccgattcaccatctccagggacaacggcaagaacacggtgtatctgcaaatggacagcctgaaacctgaggacacggccgtttattactgtgcagccccaaagtttcgttactactttagcacctctccaggtgattttgattcctggggccaggggacccaggtcaccgtctcctcagcggccgcatacccgtacgacgttccggactacggttcccaccaccatcaccatcac

SEQ ID NO: 113 - protein, 135 aa, synthetic construct
QVQLQESGGGLVQVGGSLRLSCAASGSILSINAMGWYRQAPGKQRELVAAITSGGSTNYADSVKGRFTISRDNAKNMLYLQMNSLKPEDTAVYYCHVVTGWGPLDWGQGTQVTVSSAAAYPYDVPDYGSHHHHHH

SEQ ID NO: 114 - DNA, 405 bp, synthetic construct
  source 1..405: mol_type = other DNA
caggtgcagctgcaggagtctgggggaggcttggtgcaggttggggggtctctacgactctcctgtgcagcctctggaagcatcctcagtatcaatgccatgggctggtaccgccaggctccagggaagcagcgcgagttggtcgcagctattactagtggtggtagcacaaactatgcagactccgtgaagggccgattcaccatctccagagacaacgccaagaacatgctatatctgcaaatgaacagcctgaaacctgaggacacagccgtctattactgccacgtcgttacgggttggggtcccctagactggggccaggggacccaggtcaccgtctcctcagcggccgcatacccgtacgacgttccggactacggttcccaccaccatcaccatcac

SEQ ID NO: 115 - protein, 137 aa, synthetic construct
QVQLQESGGGLVQAGGSLRLSCAASGFTLSSNDMGWYRQAPGKQRELVATITSGLSTNYADSVKGRFTISRDNAKNTVFLQMNSLKIEDTAVYYCEVERGWGPPRDYWGHGTQVTVSSAAAYPYDVPDYGSHHHHHH

SEQ ID NO: 116 - DNA, 411 bp, synthetic construct
  source 1..411: mol_type = other DNA
caggtgcagctgcaggagtctgggggaggcttggtgcaagctggggggtctctgagactctcctgcgcagcctctggatttaccttaagtagcaatgacatgggctggtaccgccaggctccagggaagcagcgcgaattggtcgcaactattactagtggtctgagcacaaactatgcagactccgtgaagggccgattcaccatctccagagacaacgccaagaacacggtgtttctgcaaatgaatagcctgaaaattgaggacacagccgtctattactgtgaggtagagaggggttggggaccgccgagggactactggggccacgggacccaggtcaccgtctcctcagcggccgcatacccgtacgacgttccggactacggttcccaccaccatcaccatcac

SEQ ID NO: 117 - protein, 142 aa, synthetic construct
QVQLQESGGGLVQAGGSLRLSCAASGRAFSSYVMGWFRQAPGKEREFVAAIHWTGAATVYVDSVKGRFAISRHNAKNTVYLEMNSLKPEDTAVYYCAADPSGSYWPPKRYDYWGQGTQVTVSSAAAYPYDVPDYGSHHHHHH

SEQ ID NO: 118 - DNA, 426 bp, synthetic construct
  source 1..426: mol_type = other DNA
caggtgcagctgcaggagtctggaggaggattggtgcaggctgggggctctctgagactctcctgtgcagcctctggacgcgccttcagtagttatgttatgggctggtttcgccaggctccaggaaaggagcgtgagtttgtagcagctattcattggactggtgctgctacagtctatgtagactccgtgaagggtcgattcgccatctccagacacaacgccaagaacacggtgtatctggaaatgaacagcctgaaacctgaggacacggccgtttattactgtgcagcagaccctagcggtagttactggccccccaagaggtatgactactggggccaggggacccaggtcaccgtctcctcagcggccgcatacccgtacgacgttccggactacggttcccaccaccatcaccatcac

SEQ ID NO: 119 - protein, 143 aa, synthetic construct
QVQLQESGGGLVRAGDSLRLSCAVSGRTFSSVAMGWFRQAPGKEREFVAFISGSTGSVTYYADSVKGRFAISRDNAKNTVYLQMNSLKPEDTAVYDCAAKFTGTFNYQGLYDYWGQGTQVTVSSAAAYPYDVPDYGSHHHHHH

SEQ ID NO: 120 - DNA, 429 bp, synthetic construct
  source 1..429: mol_type = other DNA
caggtgcagctgcaggagtctgggggaggattggtccgcgctggggactctctgagactctcctgtgcagtctctggacgcaccttcagtagcgttgccatgggctggttccgccaggctccagggaaggagcgtgaatttgtagcgtttattagcgggagtactggtagtgttacatactatgcagactccgtgaagggccgattcgccatctccagagacaacgccaagaacacggtgtatctacaaatgaacagcctgaagcctgaggacacggccgtttatgactgtgcagctaagtttactggtactttcaactaccaaggtctatatgactactggggccaggggacccaggtcaccgtctcctcagcggccgcatacccgtacgacgttccggactacggttcccaccaccatcaccatcac

SEQ ID NO: 121 - protein, 145 aa, synthetic construct
QVQLQESGGGLVQAGGSLRLSCAASGRTFSRYAMGWFRQAPGKERESVAAINWSGTSIFYANSVEGRFTISRDNAKNTVYLQLNSLKPEDTAVYYCAADLVRNYRLGWGDGVYDHYGQGTQVTVSSAAAYPYDVPDYGSHHHHHH

SEQ ID NO: 122 - DNA, 435 bp, synthetic construct
  source 1..435: mol_type = other DNA
caggtgcagctgcaggagtctgggggaggattggtgcaggctgggggctctctgagactctcctgtgcagcctctggacgcaccttcagtaggtatgctatgggctggttccgccaggctccagggaaggagcgtgagtctgtagcagctattaattggagtggtactagcatattctatgcaaactccgtggagggccgattcaccatctccagagacaacgccaagaacacggtgtatctgcaactgaacagcctgaaacctgaggacacggccgtttattactgtgcagcagacttggttcggaactacagattgggctggggagatggcgtctatgaccactacggccaggggacccaggtcaccgtctcctcagcggccgcatacccgtacgacgttccggactacggttcccaccaccatcaccatcac

SEQ ID NO: 123 - protein, 135 aa, synthetic construct
QVQLQESGGGLVQAGGSLRLSCAASGRTFSRYAMGWFRQAPGKERESVAAINWSGTSIFYANSVEGRFTISRDNAKNTVYLQLNSLKPEDTAVYYCGVVLNGMPYWGRGTQVTVSSAAAYPYDVPDYGSHHHHHH

SEQ ID NO: 124 - DNA, 405 bp, synthetic construct
  source 1..405: mol_type = other DNA
caggtgcagctgcaggagtctgggggagggctggtgcaggctgggggctctctgagactctcctgtgcagcctctggacgcaccttcagtaggtatgctatgggctggttccgccaggctccagggaaggagcgtgagtctgtagcagctattaattggagtggtactagcatattctatgcaaactccgtggagggccgattcaccatctccagagacaacgccaagaacacggtgtatctgcaactgaacagcctgaaacctgaggacacggccgtttattactgtggagttgtcctgaacggcatgccctactggggcagagggacccaggtcaccgtctcctcagcggccgcatacccgtacgacgttccggactacggttcccaccaccatcaccatcac

SEQ ID NO: 125 - protein, 137 aa, synthetic construct
QVQLQESGGGLVQAGGSLRLSCAASGSIFSINTMGWYRQAPGKQRELVAAITSGGSTNYADSVKGRFTISRDNAKNTVHLQMNSLKPEDTAVYYCNVVEGWGPPVGYWGQGTQVTVSSAAAYPYDVPDYGSHHHHHH

SEQ ID NO: 126 - DNA, 411 bp, synthetic construct
  source 1..411: mol_type = other DNA
caggtgcagctgcaggagtctgggggaggcttggtgcaggctggggggtctctgagactctcctgtgcagcctctggaagcatcttcagtatcaataccatgggctggtaccgccaggctccagggaagcagcgcgagttggtcgcagctattactagtggtggtagcacaaactatgcagactccgtgaagggccgattcaccatctccagagacaacgccaaaaacacggtacatctgcaaatgaacagcctgaaacctgaggacacagccgtctattactgtaatgtagttgagggttggggaccacctgtaggctactggggccaggggacccaggtcaccgtctcctcagcggccgcatacccgtacgacgttccggactacggttcccaccaccatcaccatcac

SEQ ID NO: 127 - protein, 137 aa, synthetic construct
QVQLQESGGGLVQAGGSLRVSCEVSGIMFGINTMGWYRQAPGKQRELVAHITSSGSTNYADSVKGRFTISRDNAKKTVYLQMNSLKPEDTAVYYCNVVAGWGPPPGYWGQGTQVTVSSAAAYPYDVPDYGSHHHHHH

SEQ ID NO: 128 - DNA, 411 bp, synthetic construct
  source 1..411: mol_type = other DNA
caggtgcagctgcaggagtctggaggaggcttggtgcaggctggggggtctctgagagtctcctgtgaagtctctggaatcatgtttggaattaataccatgggctggtaccgccaggctccagggaagcagcgcgaattggtcgcacacatcactagtagtggtagcacaaattatgcagactccgtgaagggccgattcaccatctccagagacaacgccaagaaaacggtgtatctgcaaatgaacagtctgaaacctgaggacacagccgtgtactactgcaatgtagttgcaggttggggtccgccacccggctactggggccaggggacccaggtcaccgtctcctcagcggccgcatacccgtacgacgttccggactacggttcccaccaccatcaccatcac

SEQ ID NO: 129 - DNA, 26 bp, synthetic construct
  source 1..26: mol_type = other DNA
acaggtacgttaatagttaatagcgt

SEQ ID NO: 130 - DNA, 22 bp, synthetic construct
  source 1..22: mol_type = other DNA
atattgcagcagtacgcacaca

(19) United States
(12) Patent Application Publication
(10) Pub. No.: US 2024/0301039 A1
(43) Pub. Date: Sep. 12, 2024

(54) Compositions that Block Activation of the Sars-CoV-2 Replication and Transcription Complex (RTC) and Methods of Use Thereof

(71) Applicants: New York University in Abu Dhabi Corporation, Abu Dhabi (AE); ISAR Bioscience GmbH, Planegg (DE)

(72) Inventors: Piergiorgio Percipalle, Abu Dhabi (AE); Gennaro Esposito, Abu Dhabi (AE); Hans-Peter Holthoff, Neuried (DE); Sabrina C. Desbordes, Munich (DE)

(21) Appl. No.: 18/606,626

(22) Filed: Mar. 15, 2024

Related U.S. Application Data
(63) Continuation of application No. PCT/IB22/00531, filed on Sep. 15, 2022.
(60) Provisional application No. 63/244,389, filed on Sep. 15, 2021.

(51) Int. Cl. C07K 16/10; A61K 39/00; A61K 48/00; A61P 31/14; G01N 33/569

(52) U.S. Cl. CPC C07K 16/1003; A61P 31/14; G01N 33/56983; A61K 2039/55555; A61K 48/00; C07K 2317/565; C07K 2317/569; C07K 2317/76; C07K 2317/92; G01N 2469/10

(57) ABSTRACT

The invention provides nanobodies that bind with high affinity to SARS-CoV-2 non-structural protein (Nsp), as well and compositions comprising the identified nanobodies and methods of use thereof to block activation of SARS-CoV-2 viral replication, and for the treatment or prevention of COVID-19.

### CROSS REFERENCE TO RELATED APPLICATIONS

This application claims priority to U.S. Provisional Application No. 63/244,389, filed Sep. 15, 2021 which is hereby incorporated by reference herein in its entirety.

### BACKGROUND OF THE INVENTION

Coronaviruses constitute the subfamily Orthocoronavirinae, in the family Coronaviridae, order Nidovirales, and realm Riboviria. They are enveloped viruses with a positive-sense single-stranded RNA genome and a nucleocapsid of helical symmetry. The genome size of coronaviruses ranges from approximately 26 to 32 kilobases, one of the largest among RNA viruses. A novel Coronavirus (SARS-CoV-2) has recently emerged and its outbreak has caused a global pandemic (COVID-19) resulting in hundreds of millions of infections and more than a million deaths worldwide due to mild to lethal respiratory tract infections.

The RNA-based genome of the newly emerged SARS-CoV-2 encodes 29 proteins, including so-called non-structural proteins (NSPs) required for viral function and replication. Among these proteins, the RNA-dependent RNA polymerase (RdRp, also named NSP12) is necessary for coronaviral replication and forms a complex with three other viral proteins NSP7, NSP8 and NSP9. Formation of this complex is required to make new copies of the viral genome. The recently solved structure of the replication complex including NSP12, NSP7, NSP8 and NSP9 by cryo-EM shows that NSP9 is required for assembly of a functional replication complex. Selective targeting of NSP9 is, therefore, likely to inhibit viral replication. Accordingly, there exists a need for a solid base for the rational design of new antiviral therapeutics to block Sars-CoV-2 replication and stop the Covid-19 pandemics. In fact, although several vaccines have recently become available for prevention, the onset of several variants of the virus makes it is imperative to design a repertoire of compounds/reagents with antiviral activity that may balance possible efficacy. The present invention meets this need.

### SUMMARY

In one embodiment, the invention relates to an isolated antibody or antibody fragment that specifically binds to a SARS-CoV-2 non-structural protein. In one embodiment, the SARS-CoV-2 non-structural protein is Nsp7, Nsp8, Nsp9, Nsp12 or Nsp13.

In one embodiment, the antibody or antibody fragment is a monoclonal antibody, a polyclonal antibody, a single chain antibody, an immunoconjugate, a glycoengineered antibody, and a bispecific antibody or other multi-specific antibody. In one embodiment, the antibody is a single chain antibody. In one embodiment, the single chain antibody is a nanobody. In one embodiment, the nanobody is specific for binding to Nsp9.

In one embodiment, the antibody comprises at least one of a CDR1 of SEQ ID NO:1, a CDR2 of SEQ ID NO:2 and a CDR3 of SEQ ID NO:3. In one embodiment, the antibody comprises at least one of a CDR1 of SEQ ID NO:9, a CDR2 of SEQ ID NO:10 and a CDR3 of SEQ ID NO:11. In one embodiment, the antibody comprises at least one a CDR1 of SEQ ID NO:17, a CDR2 of SEQ ID NO: 18 and a CDR3 of SEQ ID NO: 19. In one embodiment, the antibody comprises at least one of a CDR1 of SEQ ID NO:25, a CDR2 of SEQ ID NO:26 and a CDR3 of SEQ ID NO:27. In one embodiment, the antibody comprises at least one of a CDR1 of SEQ ID NO:33, a CDR2 of SEQ ID NO:34 and a CDR3 of SEQ ID NO:35. In one embodiment, the antibody comprises at least one of a CDR1 of SEQ ID NO:41, a CDR2 of SEQ ID NO:42 and a CDR3 of SEQ ID NO:43. In one embodiment, the antibody comprises at least one of a CDR1 of SEQ ID NO:49, a CDR2 of SEQ ID NO:50 and a CDR3 of SEQ ID NO:51. In one embodiment, the antibody comprises at least one of a CDR1 of SEQ ID NO:57, a CDR2 of SEQ ID NO:58 and a CDR3 of SEQ ID NO:59.

In one embodiment, the antibody comprises each of a CDR1 of SEQ ID NO:1, a CDR2 of SEQ ID NO:2 and a CDR3 of SEQ ID NO:3. In one embodiment, the antibody comprises each of a CDR1 of SEQ ID NO:9, a CDR2 of SEQ ID NO: 10 and a CDR3 of SEQ ID NO:11. In one embodiment, the antibody comprises each of a CDR1 of SEQ ID NO:17, a CDR2 of SEQ ID NO: 18 and a CDR3 of SEQ ID NO:19. In one embodiment, the antibody comprises each of a CDR1 of SEQ ID NO:25, a CDR2 of SEQ ID NO:26 and a CDR3 of SEQ ID NO:27. In one embodiment, the antibody comprises each of a CDR1 of SEQ ID NO:33, a CDR2 of SEQ ID NO:34 and a CDR3 of SEQ ID NO:35. In one embodiment, the antibody comprises each of a CDR1 of SEQ ID NO:41, a CDR2 of SEQ ID NO:42 and a CDR3 of SEQ ID NO:43. In one embodiment, the antibody comprises each of a CDR1 of SEQ ID NO:49, a CDR2 of SEQ ID NO:50 and a CDR3 of SEQ ID NO:51. In one embodiment, the antibody comprises each of a CDR1 of SEQ ID NO:57, a CDR2 of SEQ ID NO:58 and a CDR3 of SEQ ID NO:59.

In one embodiment, the antibody comprises an amino acid sequence of SEQ ID NO:4, SEQ ID NO:12, SEQ ID NO:20, SEQ ID NO:28, SEQ ID NO:36, SEQ ID NO:44, SEQ ID NO:52, or SEQ ID NO:60.

In one embodiment, the invention relates to a composition comprising an antibody or antibody fragment that specifically binds to a SARS-CoV-2 non-structural protein. In one embodiment, the SARS-CoV-2 non-structural protein is Nsp7, Nsp8, Nsp9, Nsp12 or Nsp13.

In one embodiment, the composition comprises a nanobody specific for binding to Nsp9. In one embodiment, the nanobody comprises at least one of a CDR1 of SEQ ID NO: 1, a CDR2 of SEQ ID NO:2 and a CDR3 of SEQ ID NO:3. In one embodiment, the nanobody comprises at least one of a CDR1 of SEQ ID NO:9, a CDR2 of SEQ ID NO: 10 and a CDR3 of SEQ ID NO:11. In one embodiment, the nanobody comprises at least one a CDR1 of SEQ ID NO: 17, a CDR2 of SEQ ID NO: 18 and a CDR3 of SEQ ID NO: 19. In one embodiment, the nanobody comprises at least one of a CDR1 of SEQ ID NO:25, a CDR2 of SEQ ID NO:26 and a CDR3 of SEQ ID NO:27. In one embodiment, the nanobody comprises at least one of a CDR1 of SEQ ID NO:33, a CDR2 of SEQ ID NO:34 and a CDR3 of SEQ ID NO:35. In one embodiment, the nanobody comprises at least one of a CDR1 of SEQ ID NO:41, a CDR2 of SEQ ID NO:42 and a CDR3 of SEQ ID NO:43. In one embodiment, the nanobody comprises at least one of a CDR1 of SEQ ID NO:49, a CDR2 of SEQ ID NO:50 and a CDR3 of SEQ ID NO:51. In one embodiment, the nanobody comprises at least one of a CDR1 of SEQ ID NO:57, a CDR2 of SEQ ID NO:58 and a CDR3 of SEQ ID NO:59.

In one embodiment, the nanobody comprises each of a CDR1 of SEQ ID NO: 1, a CDR2 of SEQ ID NO:2 and a CDR3 of SEQ ID NO:3. In one embodiment, the nanobody comprises each of a CDR1 of SEQ ID NO:9, a CDR2 of SEQ ID NO: 10 and a CDR3 of SEQ ID NO: 11. In one embodiment, the nanobody comprises each of a CDR1 of SEQ ID NO:17, a CDR2 of SEQ ID NO: 18 and a CDR3 of SEQ ID NO:19. In one embodiment, the nanobody comprises each of a CDR1 of SEQ ID NO:25, a CDR2 of SEQ ID NO:26 and a CDR3 of SEQ ID NO:27. In one embodiment, the nanobody comprises each of a CDR1 of SEQ ID NO:33, a CDR2 of SEQ ID NO:34 and a CDR3 of SEQ ID NO:35. In one embodiment, the nanobody comprises each of a CDR1 of SEQ ID NO:41, a CDR2 of SEQ ID NO:42 and a CDR3 of SEQ ID NO:43. In one embodiment, the nanobody comprises each of a CDR1 of SEQ ID NO:49, a CDR2 of SEQ ID NO:50 and a CDR3 of SEQ ID NO:51. In one embodiment, the nanobody comprises each of a CDR1 of SEQ ID NO:57, a CDR2 of SEQ ID NO:58 and a CDR3 of SEQ ID NO:59.

In one embodiment, the nanobody comprises an amino acid sequence of SEQ ID NO:4, SEQ ID NO: 12, SEQ ID NO:20, SEQ ID NO:28, SEQ ID NO:36, SEQ ID NO:44, SEQ ID NO:52, or SEQ ID NO:60.

In one embodiment, the invention relates to a nucleic acid molecule comprising a nucleotide sequence encoding an antibody or antibody fragment that specifically binds to a SARS-CoV-2 non-structural protein. In one embodiment, the SARS-CoV-2 non-structural protein is Nsp7, Nsp8, Nsp9, Nsp12 or Nsp13.

In one embodiment, the nucleic acid molecule comprises nucleotide monomer units selected from RNA, DNA and chemically modified nucleotide monomer units.

In one embodiment, the nucleic acid molecule comprises RNA nucleotide monomer units and further comprises one or more nucleotide monomer units selected from DNA and chemically modified nucleotide monomer units. In one embodiment, the nucleic acid molecule comprises an RNA. In one embodiment, the nucleic acid molecule comprises a 5′-CAP and/or a poly-A tail.

In one embodiment, the nucleic acid molecule encodes a nanobody specific for binding to Nsp9. In one embodiment, the nanobody comprises at least one of a CDR1 of SEQ ID NO:1, a CDR2 of SEQ ID NO:2 and a CDR3 of SEQ ID NO:3. In one embodiment, the nanobody comprises at least one of a CDR1 of SEQ ID NO:9, a CDR2 of SEQ ID NO:10 and a CDR3 of SEQ ID NO:11. In one embodiment, the nanobody comprises at least one a CDR1 of SEQ ID NO: 17, a CDR2 of SEQ ID NO: 18 and a CDR3 of SEQ ID NO: 19. In one embodiment, the nanobody comprises at least one of a CDR1 of SEQ ID NO:25, a CDR2 of SEQ ID NO:26 and a CDR3 of SEQ ID NO:27. In one embodiment, the nanobody comprises at least one of a CDR1 of SEQ ID NO:33, a CDR2 of SEQ ID NO:34 and a CDR3 of SEQ ID NO:35. In one embodiment, the nanobody comprises at least one of a CDR1 of SEQ ID NO:41, a CDR2 of SEQ ID NO:42 and a CDR3 of SEQ ID NO:43. In one embodiment, the nanobody comprises at least one of a CDR1 of SEQ ID NO:49, a CDR2 of SEQ ID NO:50 and a CDR3 of SEQ ID NO:51. In one embodiment, the nanobody comprises at least one of a CDR1 of SEQ ID NO:57, a CDR2 of SEQ ID NO:58 and a CDR3 of SEQ ID NO:59.

In one embodiment, the nanobody comprises each of a CDR1 of SEQ ID NO:1, a CDR2 of SEQ ID NO:2 and a CDR3 of SEQ ID NO:3. In one embodiment, the nanobody comprises each of a CDR1 of SEQ ID NO:9, a CDR2 of SEQ ID NO: 10 and a CDR3 of SEQ ID NO:11. In one embodiment, the nanobody comprises each of a CDR1 of SEQ ID NO: 17, a CDR2 of SEQ ID NO: 18 and a CDR3 of SEQ ID NO: 19. In one embodiment, the nanobody comprises each of a CDR1 of SEQ ID NO:25, a CDR2 of SEQ ID NO:26 and a CDR3 of SEQ ID NO:27. In one embodiment, the nanobody comprises each of a CDR1 of SEQ ID NO:33, a CDR2 of SEQ ID NO:34 and a CDR3 of SEQ ID NO:35. In one embodiment, the nanobody comprises each of a CDR1 of SEQ ID NO:41, a CDR2 of SEQ ID NO:42 and a CDR3 of SEQ ID NO:43. In one embodiment, the nanobody comprises each of a CDR1 of SEQ ID NO:49, a CDR2 of SEQ ID NO:50 and a CDR3 of SEQ ID NO:51. In one embodiment, the nanobody comprises each of a CDR1 of SEQ ID NO:57, a CDR2 of SEQ ID NO:58 and a CDR3 of SEQ ID NO:59.

In one embodiment, the nanobody comprises an amino acid sequence of SEQ ID NO:4, SEQ ID NO: 12, SEQ ID NO:20, SEQ ID NO:28, SEQ ID NO:36, SEQ ID NO:44, SEQ ID NO:52, or SEQ ID NO:60.

In one embodiment, the nucleic acid molecule comprises at least one nucleotide sequence of SEQ ID NO:5, SEQ ID NO:6 and SEQ ID NO:7, encoding CDR1, CDR2 and CDR3 respectively. In one embodiment, the nucleic acid molecule comprises at least one nucleotide sequence of SEQ ID NO: 13, SEQ ID NO: 14 and SEQ ID NO:15, encoding CDR1, CDR2 and CDR3 respectively. In one embodiment, the nucleic acid molecule comprises at least one nucleotide sequence of SEQ ID NO:21, SEQ ID NO:22 and SEQ ID NO:23, encoding CDR1, CDR2 and CDR3 respectively. In one embodiment, the nucleic acid molecule comprises at least one nucleotide sequence of SEQ ID NO:29, SEQ ID NO:30 and SEQ ID NO:31, encoding CDR1, CDR2 and CDR3 respectively. In one embodiment, the nucleic acid molecule comprises at least one nucleotide sequence of SEQ ID NO:37, SEQ ID NO:38 and SEQ ID NO:39, encoding CDR1, CDR2 and CDR3 respectively. In one embodiment, the nucleic acid molecule comprises at least one nucleotide sequence of SEQ ID NO:45, SEQ ID NO:46 and SEQ ID NO:47, encoding CDR1, CDR2 and CDR3 respectively. In one embodiment, the nucleic acid molecule comprises at least one nucleotide sequence of SEQ ID NO:53, SEQ ID NO:54 and SEQ ID NO:55, encoding CDR1, CDR2 and CDR3 respectively. In one embodiment, the nucleic acid molecule comprises at least one nucleotide sequence of SEQ ID NO:61, SEQ ID NO:62 and SEQ ID NO:63, encoding CDR1, CDR2 and CDR3 respectively.

In one embodiment, the nucleic acid molecule comprises each of SEQ ID NO:5, SEQ ID NO:6 and SEQ ID NO: 7, encoding CDR1, CDR2 and CDR3 respectively. In one embodiment, the nucleic acid molecule comprises each of SEQ ID NO: 13, SEQ ID NO:14 and SEQ ID NO:15, encoding CDR1, CDR2 and CDR3 respectively. In one embodiment, the nucleic acid molecule comprises each of SEQ ID NO:21, SEQ ID NO:22 and SEQ ID NO:23, encoding CDR1, CDR2 and CDR3 respectively. In one embodiment, the nucleic acid molecule comprises each of SEQ ID NO:29, SEQ ID NO:30 and SEQ ID NO:31, encoding CDR1, CDR2 and CDR3 respectively. In one embodiment, the nucleic acid molecule comprises each of SEQ ID NO:37, SEQ ID NO:38 and SEQ ID NO:39, encoding CDR1, CDR2 and CDR3 respectively. In one embodiment, the nucleic acid molecule comprises each of SEQ ID NO:45, SEQ ID NO:46 and SEQ ID NO:47, encoding CDR1, CDR2 and CDR3 respectively. In one embodiment, the nucleic acid molecule comprises each of SEQ ID NO:53, SEQ ID NO:54 and SEQ ID NO:55, encoding CDR1, CDR2 and CDR3 respectively. In one embodiment, the nucleic acid molecule comprises each of SEQ ID NO:61, SEQ ID NO:62 and SEQ ID NO:63, encoding CDR1, CDR2 and CDR3 respectively.

In one embodiment, the nucleic acid molecule comprises SEQ ID NO:8, SEQ ID NO: 16, SEQ ID NO:24, SEQ ID NO:32, SEQ ID NO:40, SEQ ID NO:48, SEQ ID NO:56, or SEQ ID NO:64.

In one embodiment, the invention relates to a composition comprising a nucleic acid molecule comprising a nucleotide sequence encoding an antibody or antibody fragment that specifically binds to a SARS-CoV-2 non-structural protein. In one embodiment, the SARS-CoV-2 non-structural protein is Nsp7, Nsp8, Nsp9, Nsp12 or Nsp13. In one embodiment, the composition comprises a nucleic acid molecule encoding a nanobody specific for binding to Nsp9.

In one embodiment, the composition comprises a lipid nanoparticle (LNP) comprising the nucleic acid molecule. In one embodiment, the lipid nanoparticle comprises one or more lipids.

In one embodiment, the lipid nanoparticle comprises at least one ionizable cationic lipid. In one embodiment, the ionizable cationic lipid is C12-200.

In one embodiment, the lipid nanoparticle comprises at least one neutral amphoteric or zwitterionic lipid. In one embodiment, the neutral amphoteric or zwitterionic lipid comprises DOPE.

In one embodiment, the lipid nanoparticle comprises cholesterol.

In one embodiment, the lipid nanoparticle comprises at least one non-ionic lipid. In one embodiment, the at least one non-ionic lipid comprises at least one PEGylated non-ionic lipid. In one embodiment, the PEGylated non-ionic lipid comprises DMG-PEG.

In one embodiment, the lipid nanoparticle comprises at least one quaternary ammonium cationic lipid. In one embodiment, the at least one quaternary ammonium cationic lipid comprises DOTAP.

In one embodiment, the lipid nanoparticle comprises two or more of an ionizable cationic lipid, a neutral amphoteric or zwitterionic lipid, a non-ionic lipid, cholesterol, and a quaternary ammonium cationic lipid. In one embodiment, the lipid nanoparticle comprises an ionizable cationic lipid, a neutral amphoteric or zwitterionic lipid, a non-ionic lipid, cholesterol, and a quaternary ammonium cationic lipid.

In one embodiment, the lipid nanoparticle comprises C12-200, DOPE, cholesterol, DMG-PEG and DOTAP.

In one embodiment, the composition comprises an LNP comprising an RNA molecule encoding the antibody or antibody fragment.

In one embodiment, the invention relates to an expression vector comprising a nucleic acid molecule comprising a nucleotide sequence encoding an antibody or antibody fragment that specifically binds to a SARS-CoV-2 non-structural protein. In one embodiment, the SARS-CoV-2 non-structural protein is Nsp7, Nsp8, Nsp9, Nsp12 or Nsp13. In one embodiment, the expression vector comprises a nucleic acid molecule encoding a nanobody specific for binding to Nsp9.

In one embodiment, the invention relates to a host cell comprising an expression vector comprising a nucleic acid molecule comprising a nucleotide sequence encoding an antibody or antibody fragment that specifically binds to a SARS-CoV-2 non-structural protein. In one embodiment, the SARS-CoV-2 non-structural protein is Nsp7, Nsp8, Nsp9, Nsp12 or Nsp13. In one embodiment, the host cell comprises an expression vector comprising a nucleic acid molecule encoding a nanobody specific for binding to Nsp9.

In one embodiment, the invention relates to a method of preventing SARS-CoV-2 viral replication in a subject in need thereof, the method comprising the step of administering an antibody or antibody fragment that specifically binds to a SARS-CoV-2 non-structural protein, or a composition comprising the same, to a subject in need thereof. In one embodiment, the antibody or fragment thereof is a nanobody specific for binding to Nsp9.

In one embodiment, the invention relates to a method of preventing SARS-CoV-2 viral replication in a subject in need thereof, the method comprising the step of administering a nucleic acid molecule encoding an antibody or antibody fragment that specifically binds to a SARS-CoV-2 non-structural protein, or a composition comprising the same, to a subject in need thereof. In one embodiment, the antibody or fragment thereof is a nanobody specific for binding to Nsp9.

In one embodiment, the invention relates to a method of treating or preventing a disease or disorder associated with SARS-CoV-2 infection in a subject in need thereof, the method comprising the step of administering an antibody or antibody fragment that specifically binds to a SARS-CoV-2 non-structural protein, or a composition comprising the same, to a subject in need thereof. In one embodiment, the disease associated with SARS-CoV-2 infection comprises COVID-19.

In one embodiment, the invention relates to a method of treating or preventing a disease or disorder associated with SARS-CoV-2 infection in a subject in need thereof, the method comprising the step of administering a nucleic acid molecule encoding an antibody or antibody fragment that specifically binds to a SARS-CoV-2 non-structural protein, or a composition comprising the same, to a subject in need thereof. In one embodiment, the disease associated with SARS-CoV-2 infection comprises COVID-19.

In one embodiment, the invention relates to a method of detecting a SARS-CoV-2 Nsp in a sample, the method comprising: a) contacting the sample with an antibody or antibody fragment that specifically binds to a SARS-CoV-2 non-structural protein, or a composition comprising the same, and b) detecting binding of the antibody or antibody fragment to the target SARS-CoV-2 Nsp.

In one embodiment, the invention relates to a method of diagnosing SARS-CoV-2 infection in a subject in need thereof, the method comprising the steps of: a) contacting a biological sample of the subject with an antibody or antibody fragment that specifically binds to a SARS-CoV-2 non-structural protein, or a composition comprising the same, b) determining the presence of the SARS-CoV-2 Nsp in the biological sample of the subject, and c) diagnosing the subject with a SARS-CoV-2 infection when SARS-CoV-2 Nsp is detected the in the biological sample of the subject. In one embodiment, the method further comprises a step of administering a treatment to the subject that was diagnosed as having SARS-CoV-2 infection.

### BRIEF DESCRIPTION OF THE DRAWINGS

The foregoing summary, as well as the following detailed description of preferred embodiments of the invention, will be better understood when read in conjunction with the appended drawings. For the purpose of illustrating the invention, there are shown in the drawings embodiments which are presently preferred. It should be understood, however, that the invention is not limited to the precise arrangements and instrumentalities of the embodiments shown in the drawings.

FIG. 1 depicts a representative size exclusion chromatogram profile of a nanobody. The highlighted peak represents the elution of the nanobody and the respective fractions collected. The example presented here depicts the size exclusion chromatogram of nanobody 2INS64.

FIG. 2 depicts the production yields of the Nsp9-specific nanobodies in the expression vector pHEN6.

FIG. 3 depicts SDS-PAGE (top panels) and Western blot (bottom panels) analysis of the 8 purified nanobodies. 1 & 10-PageRuler™ Prestained protein Ladder, 2 & 11-2NSP23, 3 & 12-2NSP90, 4 & 13-3NSP52, 5 & 14-3NSP78, 6 & 15-2INS27, 7 & 16-2INS45, 8 & 17-2INS64, 9 & 18-2INS69.

FIG. 4 depicts an amino acid sequence analysis of the nanobodies using the ProtParam tool which allows the prediction of several theorical proprieties. MW, Molecular Weight; pI, isoelectric point; ε, extinction coefficient.

FIG. 5A and FIG. 5B depict exemplary experimental data demonstrating molecular dynamics simulations of wild-type and mutant Nsp9. FIG. 5A depicts a model of triSer-Nsp9 with red highlights at the positions of the three serines replacing the cysteines in the wild-type sequence. FIG. 5B depicts the average pairwise RMSD values upon monomer-monomer superposition along the sequences of the homodimeric subunits of WT SARS-CoV-2 Nsp9 and the triSer-Nsp9 mutant, calculated from snapshots selected every 1 ns from a 200 ns molecular dynamics (MD) trajectory. The two subunits of each homodimer are indicated by separate traces. Deviations were observed for one or both subunits of the wild-type homodimer at approximately residues 55-65, as well as at the N- and C-termini, corresponding to a slightly larger conformational spread of wild-type with respect to the corresponding dispersion of the mutant. Overall, however, the observation of a similar MD pattern confirmed that no major difference should occur between the parent and the mutant subunits of the homodimers, and therefore the triSer-Nsp9 was deemed suitable for raising the HCAbs response.

FIG. 6 depicts representative size exclusion chromatogram profiles of selected nanobodies. The highlighted peaks represent the elution of each nanobody and the respective fractions collected.

FIG. 7A and FIG. 7B depicts sequences, annotations and analytical characterizations of nanobodies. FIG. 7A depicts the annotated amino acid sequences of eight selected nanobodies based on their DNA sequences. Alignment and annotations were done using the ProtParam tool (web.expasy.org/protparam/). The sequence information is as follows: 2INS27 is provided in SEQ ID NO:4; 2INS45 is provided in SEQ ID NO:44; 2INS64 is provided in SEQ ID NO:36; 2INS61 is provided in SEQ ID NO:84; 3INS39 is provided in SEQ ID NO:92; 2NSP23 is provided in SEQ ID NO:28; 3NSP52 is provided in SEQ ID NO:12; 2NSP90 is provided in SEQ ID NO:20; 3NSP56 is provided in SEQ ID NO:68; 3NSP29 is provided in SEQ ID NO:76; 3NSP78 is provided in SEQ ID NO:52; 2INS69 is provided in SEQ ID NO:60. FIG. 7B depicts SDS-PAGE (top panels) and Western blot (bottom panels) analysis of the 8 purified nanobodies. Western blotting was performed with anti-His tag antibodies. For quality control, 5 μg of each purified nanobody were loaded onto two 12.5% SDS-PAGE gels for Coomassie Blue staining or for Western blotting with mouse anti-His tag antibodies detected with a goat anti-mouse-HRP antibody. All purified nanobodies revealed a single band profile on both gel and Western blot in both reducing and non-reducing conditions.

FIG. 8A through FIG. 8C depicts exemplary experimental data demonstrating that llama derived nanobodies specifically cross-react with Nsp9 in COVID-19 saliva samples. FIG. 8A depicts data demonstrating that decreasing amount of purified recombinantly expressed Nsp9 (purified rec Nsp9) pre-incubated with BSA were separated by SDS PAGE, immunostained with nanobodies 2NSP23 and 2NSP90. Detection was with HRP-conjugated secondary antibodies to 6×His tag (aHis-HRP) or to the VHH domain (aVHH-HRP). FIG. 8B depicts and exemplary RTqPCR analysis of Sars-Cov-2 N2 mRNA levels as proxy for viral load. mRNA levels were normalized against human RNase P mRNA. (FIG. 8C) 15 μg of saliva protein samples from COVID-19 negative and positive individuals were loaded together with 50 ng and 10 ng of purified NSP9 which served as a positive control. Top panel, SDS PAGE, bottom panel, corresponding immunoblots with nanobodies 2NSP23 and 2NSP90.

FIG. 9A through FIG. 9E depict exemplary experimental data characterizing the nature of the interaction of 2NSP90 and 2NSP23 with their antigen Nsp9. FIG. 9A through FIG. 9C depicts data demonstrating the ¹⁵N-¹H HSQC NMR spectrum of SARS-CoV-2 Nsp9 (138 μM in phosphate buffer, pH 7.03, 298 K). A moderately strong resolution enhancement weighing function (45°-shifted squared sinebell) was applied prior to 2D Fourier transform. For the red-highlighted region, the right panels show the difference between the signals without (FIG. 9B) and with (FIG. 9C) the same resolution enhancement as applied in (FIG. 9A). FIG. 9D depicts exemplary experimental data demonstrating that ¹⁵N-¹H HSQC NMR spectrum of SARS-CoV-2 triSer-Nsp9 (131 μM in aqueous acetate, pH 4.7, 298 K). Similar spectra are obtained also at pH 3.7 and 6.1. FIG. 9E depicts exemplary experimental data demonstrating the overlay of the HSQC maps of triSer-Nsp9 and wild-type Nsp9 from SARS-CoV-2. The missing signals in the spectrum of triSer-Nsp9 are A30, L45, L44, T67/S46, T18, G17, T19, T21, A108, C23, L69, A22 and E38, whereas the additional signals present only in the spectrum of triSer-Nsp9 are G104, G100 and G37, although this is tentative for G100 and G104.

FIG. 10A through FIG. 10D depict exemplary experimental data demonstrating rearrangement of the interface upon tetramerization. FIG. 10A depicts data demonstrating the crystal structure of SARS-CoV-2 Nsp9 tetramer (PDB ID: 7BWQ). The “*” locate fragments 67-69, 17-22 and residue 37, namely the inter-dimer contact surface that results highlighted by the different NMR pattern observed with triSer-Nsp9 and Nsp9. The arrows show the positions of G100, G104 and A108 at the intra-dimer contact surface. Also these residues exhibit a responsive pattern when comparing the spectra of the mutant and wild type. The “%” regions (fragments 30-32 and 44-46) respond with a similar pattern as the “*” regions in the mutant spectrum, most likely revealing effects that occur more distantly with respect to the contact areas. FIG. 10B depicts an overlay of the 15N-1H HSQC maps of SARS-CoV-2 Nsp9 recorded at 278 K, in the absence (black contours) and presence of 2NSP23, at protein:nanobody ratio 1:0.43 (dark grey contours) and 1:0.63 (light grey contours). FIG. 10C depicts data demonstrating the ¹⁵N-¹H HSQC spectrum of Nsp9 and 2NSP23 at protein:nanobody ratio 1:2. Similar patterns were obtained also with 2NSP90. FIG. 10D depicts data demonstrating an overlay of 15N-1H HSQC regions of Nsp9 recorded at 276 K, in the absence (black contours) and presence of 2NSP90, at protein:nanobody ratio 1:0.43 (*) and 1:0.74 (light grey contours). Analogous chemical shift changes were observed also with 2NSP23.

FIG. 11A through FIG. 11B depict exemplary experimental data demonstrating the extent of oligomerization in triSer-Nsp9. FIG. 11A depicts an aliphatic region overlay from the DOSY contour maps of SARS-CoV-2 triSer-Nsp9 (black), wild-type Nsp9 (medium grey) and hen egg white lysozyme (HEWL, light grey). The D) values are 1.07±0.01×10⁻¹⁰ m²/s and 1.13±0.02×10⁻²¹⁰ m/s for the mutant and wild-type Nsp9, respectively. For HEWL, considered as a marker, a value of 1.24±0.02×10⁻¹⁰ m²/s is obtained. Protein concentrations in H2O/D2O 95/5 were ˜130 μM for Nsp9 variants and 120 μM for HEWL. The overlay shows that the translational diffusion is slower for triSer-Nsp9 (black trace) compared to wild-type Nsp9. Considering the crystallographic Nsp9 dimensions (Zhang et al., 2020, Molecular Biomedicine, 1:5), the prolate ellipsoids of revolution representing monomers and dimers (with axial ratios 1.304 and 1.805, respectively) correspond to spheres with radius of 1.71 nm and 2.28 nm (Hansen, 2004, J. Chem. Phys, 121:9111-9115) with an expected D value of 1.06×10⁻¹⁰ m²/s and 1.41×10⁻¹⁰ m²/s. Therefore, the experimentally determined D values and the reported NMR evidence consistently indicate that the extent of oligomerization is more pronounced for triSer-Nsp9, with an essentially complete dimerization for the visible signals. Instead, the observable NMR signals of wild-type Nsp9 also bear some contribution from monomers, which indicates a difference in the extent of dimerization between triSer-Nsp9 and the natural sequence with three cysteines. FIG. 11B depicts the average pairwise RMSD values upon dimer-dimer superposition along the sequences of the homodimer subunits of SARS-CoV-2 Nsp9 and triSer-Nsp9 mutant, calculated from the snapshots selected every 1 ns from a 200 ns MD trajectory. The two subunits of each homodimer are indicated by separate traces. The average pairwise RMSD was calculated upon dimer-dimer superposition of all mutant snapshots with all wild-type snapshots. The conclusion inferred from DOSY is consistent with the MD simulation results. The average pairwise RMSD values, upon mutant versus wild-type dimer-dimer superposition, are larger than the single species counterparts at both N-terminal and C-terminal tracts, implying that wild type and mutated Nsp9 fluctuate around different conformers. As those tracts are involved at the inter-monomer interface (Zhang et al., 2020, Molecular Biomedicine, 1:5), the pairwise RMSD profile is consistent with difference in the dimerization extent between the species.

FIG. 12 depicts an overlay of the ¹⁵N-¹H HSQC maps of SARS-CoV-2 Nsp9 at 298K (black contours) and 278 K (light grey contours).

FIG. 13A through FIG. 13D depicts data demonstrating that the residues with high attenuation rates and the order of peak loss replicate the regions involved directly and indirectly in the tetramer assembly and the dimerization interface rearrangement. FIG. 13A depicts data demonstrating that the SARS-CoV-2 Nsp9 tetramer with the inter-dimer and inter-monomer contact surfaces. The e1, e2a, e2b, e3 and e4 surfaces indicate the location of the tetramer epitopes interacting with nanobodies 2NSP23 and 2NSP90. An analogous epitope pair is present on the opposite face of the tetramer. The first epitope is comprised of the surfaces e1, e2a and e2b formed by segment [Q11-M12-S13-C14] with residue L29, residue N27 and residue K86, respectively, (FIG. 13B), and the additional contributions from L45 and S46 that are already part of the tetramer interface. The second epitope is comprised of the surfaces e3 and e4 formed by the segments [D50-L51-K52-W53] (FIG. 13C) and [C73-R74-F75-V76+Y87-L88-Y89] (FIG. 13D), respectively.

FIG. 14 depicts data demonstrating the fitting of the chemical shift changes observed at the peripheral residues of SARS-CoV-2 Nsp9 upon titration with 2NSP90. As reported in main text, besides the cross-peak loss observed with titrant increase, we could also detect progressive chemical shift changes. Those titration chemical shifts involve mostly the N-terminal and C-terminal residue signals, namely A8, L9, R111 and Q113 and a couple of other locations (C73, V76). This pattern is compatible with the intermediate exchange regime observed for all the other residues of Nsp9 and may arise for intrinsically mobile molecular locations where the chemical shift is effectively averaged by the local dynamics, leading to a very small difference between the limiting chemical shift values and matching therefore local fast exchange regime. The experimental data were fitted using a Hill-Langmuir model with equation:

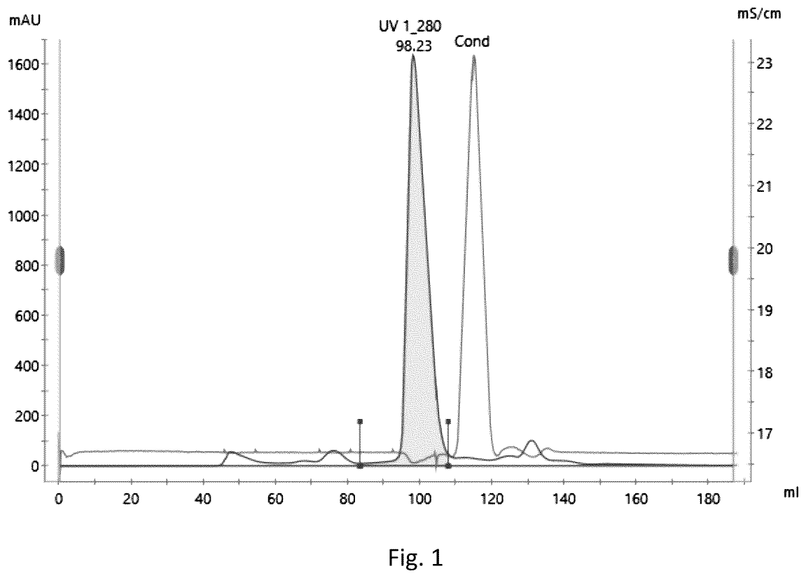

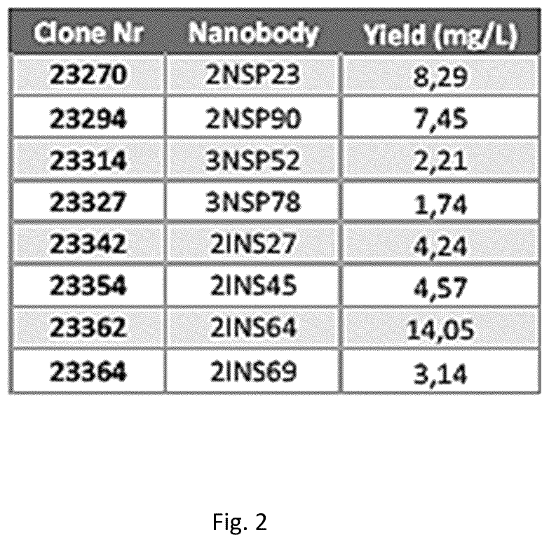

$$Δδ = \frac{{Δδ}_{max} \times {c}^{n}}{{K}_{A}^{n} + {c}^{n}}$$

where Δδ and Δδmax are the observed and maximum chemical shift change, c is the nanobody concentration, K_{A} is half occupation constant (related to the apparent dissociation constant) and n is the Hill coefficient. For A8, the fitting gave n=2.9±0.6 (p value 1.5×10⁻³) and KA=9.7 μM (p value 3.2×10 6), whereas for Q113, n=4.0±0.5 (p value 3.7×10 5) and KA=11 μM (p value 4.1×10⁻⁹). Albeit statistically significant, the fitting parameters should be regarded with some caution. This is expected due to difficulty of appreciating the limiting Δδ values and the experimental titration errors with concentrations as small as 18 μM (constant for Nsp9) and 36 μM (maximum for 2NSP90).

FIG. 15 depicts a schematic representation of SARS-CoV-2 life cycle. The coronavirus virion consists of structural proteins, namely spike (S), envelope (E), membrane (M), nucleocapsid (N) and, for some betacoronaviruses, haemagluttinin-esterase (not shown). The positive-sense, single-stranded RNA genome (+ssRNA) is encapsidated by N, whereas M and E ensure its incorporation in the viral particle during the assembly process. Coronavirus particles bind to cellular attachment factors and specific S interactions with the cellular receptors (such as angiotensin-converting enzyme 2 (ACE2)), together with host factors (such as the cell surface serine protease TMPRSS2), promote viral uptake and fusion at the cellular or endosomal membrane. Following entry, the release and uncoating of the incoming genomic RNA subject it to the immediate translation of two large open reading frames, ORF1a and ORF1b. The resulting polyproteins pp1a and pp1ab are co-translationally and post-translationally processed into the individual non-structural proteins (Nsp) that form the viral replication and transcription complex (RTC) required for replication of the viral genome. Inset, cryoEM structure of the RTC complex with the primary Nsp components, including Nsp9 and the RNA polymerase Nsp12 (Yan, L., et al., 2021. Cell 184, 184-193.e10).

FIG. 16A through FIG. 16G depicts data demonstrating that nanobody 2NSP23 targets Nsp9 and inhibits SARS-CoV-2 replication. FIG. 16A depicts data demonstrating that lipid-nanoparticles were formulated in order to contain nanobody 2NSP23 mRNA for their delivery in human cells. Schematic representation of the experimental pipeline followed to generate LNP-mRNA-2NSP23. FIG. 16B depicts data demonstrating that LNP-2NSP23 mRNA is incorporated and translated in Hek293-ACE2. LNP-2NSP23 mRNA or LNP-dTomato mRNA were added to a monolayer of confluent Hek293-ACE2 cells at concentrations of 200 μg/ml of RNA and 5 mM of lipids and incubated for 16 hours. Detection of translated mRNAs into 2NSP23 nanobody or dTomato protein was obtained by immunostaining with llama's anti-VHH antibodies or by dTomato imaging. FIG. 16C depicts data demonstrating a schematic representation of the experimental pipeline followed to establish the inhibitory role of nanobody 2NSP23 in viral replication by quantitative bioluminescence. FIG. 16D depicts data demonstrating 10\hat{ }5 HEK293-ACE2 cells were seeded in 24-wells plates and treated the next day with LNP (10 μM) and mRNA-2NSP23 (0.4 μg/ml) targeting SARS-CoV-2 Nsp9, or LNP (10 μM) and mRNA-NLP45 (0.4 μg/ml) expressing dTomato protein, as a control. Twenty-four hours after LNP-mRNA treatment, cells were infected with the Wuhan SARS-CoV2 strain engineered to express a nanoluciferase gene. FIG. 16E depicts data demonstrating a schematic representation of the experimental pipeline followed to establish the inhibitory role of nanobody 2NSP23 in viral replication by GFP imaging. FIG. 16F depicts data demonstrating 10\hat{ }5 HEK293-ACE2 cells were seeded in 24-wells plates and treated the next day with LNP (10 μM) and mRNA-2NSP23 (0.4 μg/ml) targeting SARS-CoV-2 Nsp9, or LNP (10 μM) and mRNA-NLP45 (0.4 μg/ml) targeting dTomato protein, as a control. Twenty-four hours post LNP-mRNA treatment, cells were infected with the Wuhan SARS-CoV2 strain engineered to express GFP. FIG. 16G depicts data demonstrating 10\hat{ }5 HEK293-ACE2 cells were seeded in 24-wells plates and treated the next day with LNP (10 μM) and mRNA-2NSP23 (0.4 μg/ml) targeting SARS-CoV-2 Nsp9, or LNP (10 μM) and mRNA-NLP45 (0.4 μg/ml) targeting dTomato protein, as a control. Twenty-four hours post LNP-mRNA treatment, cells were infected with indicated SARS-CoV2 strains (Wuhan) (FIG. 16G) 10\hat{ }5 HEK293-ACE2 cells were seeded in 24-wells plates and treated the next day with LNP (10 μM) and mRNA-2NSP23 (0.4 μg/ml) targeting SARS-CoV-2 Nsp9, or LNP (10 μM) and mRNA-NLP45 (0.4 μg/ml) targeting dTomato protein, as a control. Twenty-four hours post LNP-mRNA treatment, cells were infected with the SARS-CoV2 strain Wuhan.

FIG. 17 depicts data demonstrating that by targeting Nsp9, nanobody 2NSP23 inhibits replication of multiple SARS-CoV-2 variants and may serve a role as pan-inhibitor of corona viruses. Cells were incubated with LNP-mRNA-2NSP23 targeting Nsp9 or LNP-mRNA-NLP45 expressing dTomato protein as a control and then infected with the indicated SARS-CoV2 strains (Wuhan, alpha=UK=B1.1.7, Mu=B1.621, Delta=B1.617.x, and Omicron=B1.1.529). Total RNA was isolated and analyzed by qPCR to amplify and quantify the SARS-CoV-2 E gene. Relative viral RNAs were quantified over three independent experiments according to the ΔΔCt standard method. The inhibition effect of LNP-mRNA-2NSP23 targeting NSP9 on viral replication is determined relative to LNP-mRNA-NLP45 expressing dTomato protein.

### DETAILED DESCRIPTION OF THE INVENTION

The invention is based, in part on the development of nanobodies that block activation of the SARS-CoV-2 Replication and Transcription Complex (RTC). In some embodiments, the nanobody of the invention binds to SARS-CoV-2 Nsp9 and induces oligomerization of the Nsp9 protein, preventing activity of the monomeric form, thereby blocking activation of the SARS-CoV-2 RTC.

Thus, in various embodiments, the invention provides SARS-CoV-2 Nsp9 nanobodies, nucleic acid molecules encoding the SARS-CoV-2 Nsp9 nanobodies and compositions comprising the same. The invention also provides methods of use of the nanobodies and compositions of the invention for diagnosing or treating SARS-CoV-2 infection and for treating or preventing diseases and disorders associated with SARS-CoV-2 infection such as Coronavirus Disease 2019 (COVID-19).

### Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

Generally, the nomenclature used herein and the laboratory procedures in cell culture, molecular genetics, organic chemistry, and nucleic acid chemistry and hybridization are those well-known and commonly employed in the art.

Standard techniques are used for nucleic acid and peptide synthesis. The techniques and procedures are generally performed according to conventional methods in the art and various general references (e.g., Sambrook and Russell, 2012, Molecular Cloning, A Laboratory Approach, Cold Spring Harbor Press, Cold Spring Harbor, NY, and Ausubel et al., 2012, Current Protocols in Molecular Biology, John Wiley & Sons, NY), which are provided throughout this document.

The nomenclature used herein and the laboratory procedures used in analytical chemistry and organic syntheses described below are those well-known and commonly employed in the art. Standard techniques or modifications thereof are used for chemical syntheses and chemical analyses.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, or ±10%, or ±5%, or ±1%, or ±0.1% from the specified value, as such variations are appropriate to perform the disclosed methods.

The term “abnormal” when used in the context of organisms, tissues, cells or components thereof, refers to those organisms, tissues, cells or components thereof that differ in at least one observable or detectable characteristic (e.g., age, treatment, time of day, etc.) from those organisms, tissues, cells or components thereof that display the “normal” (expected/homeostatic) respective characteristic. Characteristics which are normal or expected for one cell, tissue type, or subject, might be abnormal for a different cell or tissue type.

The term “analog” as used herein generally refers to compounds that are generally structurally similar to the compound of which they are an analog, or “parent” compound. Generally analogs will retain certain characteristics of the parent compound, e.g., a biological or pharmacological activity. An analog may lack other, less desirable characteristics, e.g., antigenicity, proteolytic instability, toxicity, and the like. An analog includes compounds in which a particular biological activity of the parent is reduced, while one or more distinct biological activities of the parent are unaffected in the “analog.” As applied to polypeptides, the term “analog” may have varying ranges of amino acid sequence identity to the parent compound, for example at least about 70%, more preferably at least about 80%-85% or about 86%-89%, and still more preferably at least about 90%, about 92%, about 94%, about 96%, about 98% or about 99% of the amino acids in a given amino acid sequence the parent or a selected portion or domain of the parent. As applied to polypeptides, the term “analog” generally refers to polypeptides which are comprised of a segment of about at least 3 amino acids that has substantial identity to at least a portion of a binding domain fusion protein. Analogs typically are at least 5 amino acids long, at least 20 amino acids long or longer, at least 50 amino acids long or longer, at least 100 amino acids long or longer, at least 150 amino acids long or longer, at least 200 amino acids long or longer, and more typically at least 250 amino acids long or longer. Some analogs may lack substantial biological activity but may still be employed for various uses, such as for raising antibodies to predetermined epitopes, as an immunological reagent to detect and/or purify reactive antibodies by affinity chromatography, or as a competitive or noncompetitive agonist, antagonist, or partial agonist of a binding domain fusion protein function.

The term “antibody,” as used herein, refers to an immunoglobulin molecule which is able to specifically bind to a specific epitope of a binding partner molecule. Antibodies can be intact immunoglobulins derived from natural sources, or from recombinant sources and can be immunoreactive portions of intact immunoglobulins. The antibodies in the present invention may exist in a variety of forms including, for example, polyclonal antibodies, monoclonal antibodies, intracellular antibodies (“intrabodies”), Fv, Fab, Fab′, F(ab)2 and F(ab′)2, as well as single chain antibodies (scFv), heavy chain antibodies, such as camelid antibodies, and humanized antibodies (Harlow et al., 1999, Using Antibodies: A Laboratory Manual, Cold Spring Harbor Laboratory Press, NY; Harlow et al., 1989, Antibodies: A Laboratory Manual, Cold Spring Harbor, New York; Houston et al., 1988, Proc. Natl. Acad. Sci. USA 85:5879-5883; Bird et al., 1988, Science 242:423-426).

The term “antibody fragment” refers to at least one portion of an intact antibody and refers to the antigenic determining variable regions of an intact antibody. Examples of antibody fragments include, but are not limited to, Fab, Fab′, F(ab′)2, and Fv fragments, linear antibodies, sdAb (either V_{L} or V_{H}), camelid V_{HH} domains, scFv antibodies, and multi-specific antibodies formed from antibody fragments. The term “scFv” refers to a fusion protein comprising at least one antibody fragment comprising a variable region of a light chain and at least one antibody fragment comprising a variable region of a heavy chain, wherein the light and heavy chain variable regions are contiguously linked via a short flexible polypeptide linker, and capable of being expressed as a single chain polypeptide, and wherein the scFv retains the specificity of the intact antibody from which it was derived. Unless specified, as used herein an scFv may have the V_{L} and V_{H} variable regions in either order, e.g., with respect to the N-terminal and C-terminal ends of the polypeptide, the scFv may comprise V_{L}-linker-V_{H} or may comprise V_{H}-linker-V_{L}.

An “antibody heavy chain,” as used herein, refers to the larger of the two types of polypeptide chains present in antibody molecules in their naturally occurring conformations, and which normally determines the class to which the antibody belongs.

An “antibody light chain,” as used herein, refers to the smaller of the two types of polypeptide chains present in antibody molecules in their naturally occurring conformations. Kappa (κ) and lambda (λ) light chains refer to the two major antibody light chain isotypes.

By the term “synthetic antibody” as used herein, is meant an antibody which is generated using recombinant DNA technology, such as, for example, an antibody expressed by a bacteriophage as described herein. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

A “chimeric antibody” refers to a type of engineered antibody which contains a naturally-occurring variable region (light chain and heavy chains) derived from a donor antibody in association with light and heavy chain constant regions derived from an acceptor antibody.

A “humanized antibody” refers to a type of engineered antibody having its CDRs derived from a non-human donor immunoglobulin, the remaining immunoglobulin-derived parts of the molecule being derived from one (or more) human immunoglobulin(s). In addition, framework support residues may be altered to preserve binding affinity (see, e.g., 1989, Queen et al., Proc. Natl. Acad Sci USA, 86:10029-10032; 1991, Hodgson et al., Bio/Technology, 9:421). A suitable human acceptor antibody may be one selected from a conventional database, e.g., the KABAT database, Los Alamos database, and Swiss Protein database, by homology to the nucleotide and amino acid sequences of the donor antibody. A human antibody characterized by a homology to the framework regions of the donor antibody (on an amino acid basis) may be suitable to provide a heavy chain constant region and/or a heavy chain variable framework region for insertion of the donor CDRs. A suitable acceptor antibody capable of donating light chain constant or variable framework regions may be selected in a similar manner. It should be noted that the acceptor antibody heavy and light chains are not required to originate from the same acceptor antibody. The prior art describes several ways of producing such humanized antibodies (see for example EP-A-0239400 and EP-A-054951).

The term “donor antibody” refers to an antibody (monoclonal, and/or recombinant) which contributes the amino acid sequences of its variable regions, CDRs, or other functional fragments or analogs thereof to a first immunoglobulin partner, so as to provide the altered immunoglobulin coding region and resulting expressed altered antibody with the binding specificity and neutralizing activity characteristic of the donor antibody.

The term “acceptor antibody” refers to an antibody (monoclonal and/or recombinant) heterologous to the donor antibody, which contributes all (or any portion, but in some embodiments all) of the amino acid sequences encoding its heavy and/or light chain framework regions and/or its heavy and/or light chain constant regions to the first immunoglobulin partner. In certain embodiments a human antibody is the acceptor antibody.

“CDRs” are defined as the complementarity determining region amino acid sequences of an antibody which are the hypervariable regions of immunoglobulin heavy and light chains. See, e.g., Kabat et al., Sequences of Proteins of Immunological Interest, 4th Ed., U.S. Department of Health and Human Services, National Institutes of Health (1987). There are three heavy chain and three light chain CDRs (or CDR regions) in the variable portion of an immunoglobulin. The structure and protein folding of the antibody may mean that other residues are considered part of the binding region and would be understood to be so by a skilled person. See for example Chothia et al., (1989) Conformations of immunoglobulin hypervariable regions; Nature 342, p 877-883.

The term “framework” or “framework sequence” refers to the remaining sequences of a variable region minus the CDRs. Because the exact definition of a CDR sequence may be determined by different systems, the meaning of a framework sequence is subject to correspondingly different interpretations. The six CDRs (CDR-L1, -L2, and -L3 of light chain and CDR-H1, -H2, and -H3 of heavy chain) also divide the framework regions on the light chain and the heavy chain into four sub-regions (FR1, FR2, FR3 and FR4) on each chain, in which CDR1 is positioned between FR1 and FR2, CDR2 between FR2 and FR3, and CDR3 between FR3 and FR4. Without specifying the particular sub-regions as FR1, FR2, FR3 or FR4, a framework region, as referred by others, represents the combined FR's within the variable region of a single, naturally occurring immunoglobulin chain. An FR represents one of the four sub-regions, and FRs represents two or more of the four sub-regions constituting a framework region.

As used herein, an “immunoassay” refers to any binding assay that uses an antibody capable of binding specifically to a target molecule to detect and quantify the target molecule.

By the term “specifically binds,” as used herein with respect to an antibody, is meant an antibody which recognizes a specific binding partner molecule, but does not substantially recognize or bind other molecules in a sample. For example, an antibody or nanobody that specifically binds to a binding partner molecule from one species may also bind to that binding partner molecule from one or more species. But, such cross-species reactivity does not itself alter the classification of an antibody as specific. In another example, an antibody or nanobody that specifically binds to binding partner molecule may also bind to different allelic forms of the binding partner molecule. However, such cross reactivity does not itself alter the classification of an antibody as specific.

In some instances, the terms “specific binding” or “specifically binding”, can be used in reference to the interaction of an antibody, a protein, or a peptide with a second binding partner molecule, to mean that the interaction is dependent upon the presence of a particular structure (e.g., an antigenic determinant or epitope) on the binding partner molecule; for example, an antibody recognizes and binds to a specific protein structure rather than to proteins generally. If an antibody is specific for epitope “A”, the presence of a molecule containing epitope A (or free, unlabeled A), in a reaction containing labeled “A” and the antibody, will reduce the amount of labeled A bound to the antibody. In some instances, the terms “specific binding” and “specifically binding” refers to selective binding, wherein the antibody recognizes a sequence or conformational epitope important for the enhanced affinity of binding to the binding partner molecule.

The term “epitope” has its ordinary meaning of a site on binding partner molecule recognized by an antibody or a binding portion thereof or other binding molecule, such as, for example, an scFv. Epitopes may be molecules or segments of amino acids, including segments that represent a small portion of a whole protein or polypeptide. Epitopes may be conformational (i.e., discontinuous). That is, they may be formed from amino acids encoded by noncontiguous parts of a primary sequence that have been juxtaposed by protein folding.

The phrase “biological sample” as used herein, is intended to include any sample comprising a cell, a tissue, or a bodily fluid in which expression of a nucleic acid or polypeptide can be detected. Examples of such biological samples include but are not limited to blood, lymph, bone marrow, biopsies and smears. Samples that are liquid in nature are referred to herein as “bodily fluids.” Biological samples may be obtained from a patient by a variety of techniques including, for example, by scraping or swabbing an area or by using a needle to obtain bodily fluids. Methods for collecting various body samples are well known in the art.

As used herein, “conjugated” refers to covalent attachment of one molecule to a second molecule.

A “coding region” of a gene consists of the nucleotide residues of the coding strand of the gene and the nucleotides of the non-coding strand of the gene which are homologous with or complementary to, respectively, the coding region of an mRNA molecule which is produced by transcription of the gene.

A “coding region” of a mRNA molecule also consists of the nucleotide residues of the mRNA molecule which are matched with an anti-codon region of a transfer RNA molecule during translation of the mRNA molecule or which encode a stop codon. The coding region may thus include nucleotide residues comprising codons for amino acid residues which are not present in the mature protein encoded by the mRNA molecule (e.g., amino acid residues in a protein export signal sequence).

“Complementary” as used herein to refer to a nucleic acid, refers to the broad concept of sequence complementarity between regions of two nucleic acid strands or between two regions of the same nucleic acid strand. It is known that an adenine residue of a first nucleic acid region is capable of forming specific hydrogen bonds (“base pairing”) with a residue of a second nucleic acid region which is antiparallel to the first region if the residue is thymine or uracil. Similarly, it is known that a cytosine residue of a first nucleic acid strand is capable of base pairing with a residue of a second nucleic acid strand which is antiparallel to the first strand if the residue is guanine. A first region of a nucleic acid is complementary to a second region of the same or a different nucleic acid if, when the two regions are arranged in an antiparallel fashion, at least one nucleotide residue of the first region is capable of base pairing with a residue of the second region. Preferably, the first region comprises a first portion and the second region comprises a second portion, whereby, when the first and second portions are arranged in an antiparallel fashion, at least about 50%, and preferably at least about 75%, at least about 90%, or at least about 95% of the nucleotide residues of the first portion are capable of base pairing with nucleotide residues in the second portion. More preferably, all nucleotide residues of the first portion are capable of base pairing with nucleotide residues in the second portion.

As used herein, the term “derivative” includes a chemical modification of a polypeptide, polynucleotide, or other molecule. In the context of this invention, a “derivative polypeptide,” for example, one modified by glycosylation, pegylation, or any similar process, retains binding activity. For example, the term “derivative” of binding domain includes binding domain fusion proteins, variants, or fragments that have been chemically modified, as, for example, by addition of one or more polyethylene glycol molecules, sugars, phosphates, and/or other such molecules, where the molecule or molecules are not naturally attached to wild-type binding domain fusion proteins. A “derivative” of a polypeptide further includes those polypeptides that are “derived” from a reference polypeptide by having, for example, amino acid substitutions, deletions, or insertions relative to a reference polypeptide. Thus, a polypeptide may be “derived” from a wild-type polypeptide or from any other polypeptide. As used herein, a compound, including polypeptides, may also be “derived” from a particular source, for example from a particular organism, tissue type, or from a particular polypeptide, nucleic acid, or other compound that is present in a particular organism or a particular tissue type.

The term “DNA” as used herein is defined as deoxyribonucleic acid.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting there from. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

A disease or disorder is “alleviated” if the severity of a sign or symptom of the disease or disorder, the frequency with which such a sign or symptom is experienced by a patient, or both, is reduced.

An “effective amount” or “therapeutically effective amount” of a compound is that amount of compound which is sufficient to provide a beneficial effect to the subject to which the compound is administered.

The term “epitope” as used herein refers to a protein determinant capable of binding to an antibody. Epitopes usually consist of chemically active surface groupings of molecules such as amino acids or sugar side chains and usually have specific three dimensional structural characteristics, as well as specific charge characteristics. Conformational and non-conformational epitopes are distinguished in that the binding to the former but not the latter is lost in the presence of denaturing solvents.

The term “high affinity” for binding domain polypeptides described herein refers to a dissociation constant (Kd) of at least about 10⁻⁶M, preferably at least about 10⁻⁷M, more preferably at least about 10⁻⁸M or stronger, more preferably at least about 10⁻⁹M or stronger, more preferably at least about 10⁻¹⁰M or stronger, for example, up to 10⁻¹² M or stronger. However, “high affinity” binding can vary for other binding domain polypeptides.

The term “inhibit,” as used herein, means to suppress or block an activity or function, for example, about ten percent relative to a control value. Preferably, the activity is suppressed or blocked by 50% compared to a control value, more preferably by 75%, and even more preferably by 95%. “Inhibit,” as used herein, also means to reduce the level of a molecule, a reaction, an interaction, a gene, an mRNA, and/or a protein's expression, stability, function or activity by a measurable amount or to prevent entirely. Inhibitors are compounds that, e.g., bind to, partially or totally block activity, decrease, prevent, delay activation, inactivate, desensitize, or down regulate a protein, a gene, and an mRNA stability, expression, function and activity, e.g., antagonists.

The terms “modulator” and “modulation” of a molecule of interest, as used herein in its various forms, is intended to encompass antagonism, agonism, partial antagonism and/or partial agonism of an activity associated the protease of interest. In various embodiments, “modulators” may inhibit or stimulate protease expression or activity. Such modulators include small molecules agonists and antagonists of a protease molecule, antisense molecules, ribozymes, triplex molecules, and RNAi polynucleotides, and others.

As used herein, an “instructional material” includes a publication, a recording, a diagram, or any other medium of expression which can be used to communicate the usefulness of a compound, composition, vector, or delivery system of the invention in the kit for effecting alleviation of the various diseases or disorders recited herein. Optionally, or alternately, the instructional material can describe one or more methods of alleviating the diseases or disorders in a cell or a tissue of a mammal. The instructional material of the kit of the invention can, for example, be affixed to a container which contains the identified compound, composition, vector, or delivery system of the invention or be shipped together with a container which contains the identified compound, composition, vector, or delivery system. Alternatively, the instructional material can be shipped separately from the container with the intention that the instructional material and the compound be used cooperatively by the recipient.

“Isolated” means altered or removed from the natural state. For example, a nucleic acid or a peptide naturally present in its normal context in a living animal is not “isolated,” but the same nucleic acid or peptide partially or completely separated from the coexisting materials of its natural context is “isolated.” An isolated nucleic acid or protein can exist in substantially purified form, or can exist in a non-native environment such as, for example, a host cell.

An “isolated nucleic acid” refers to a nucleic acid segment or fragment which has been separated from sequences which flank it in a naturally occurring state, i.e., a DNA fragment which has been removed from the sequences which are normally adjacent to the fragment, i.e., the sequences adjacent to the fragment in a genome in which it naturally occurs. The term also applies to nucleic acids which have been substantially purified from other components which naturally accompany the nucleic acid, i.e., RNA or DNA or proteins, which naturally accompany it in the cell. The term therefore includes, for example, a recombinant DNA which is incorporated into a vector, into an autonomously replicating plasmid or virus, or into the genomic DNA of a prokaryote or eukaryote, or which exists as a separate molecule (i.e., as a cDNA or a genomic or cDNA fragment produced by PCR or restriction enzyme digestion) independent of other sequences. It also includes a recombinant DNA which is part of a hybrid gene encoding additional polypeptide sequence.

In the context of the present invention, the following abbreviations for the commonly occurring nucleic acid bases are used. “A” refers to adenosine, “C” refers to cytosine, “G” refers to guanosine, “T” refers to thymidine, and “U” refers to uridine. The term “polynucleotide” as used herein is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR, and the like, and by synthetic means.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

The term “conservative substitution,” when describing a polypeptide, refers to a change in the amino acid composition of the polypeptide that does not substantially alter the activity of the polypeptide, i.e., substitution of amino acids with other amino acids having similar properties. Conservative substitution tables providing functionally similar amino acids are well known in the art. The following six groups each contain amino acids that are generally understood to represent conservative substitutions for one another: (1) Alanine (A), Serine (S), Threonine (T); (2) Aspartic acid (D), Glutamic acid (E); (3) Asparagine (N), Glutamine (Q); (4) Arginine (R), Lysine (K); (5) Isoleucine (I), Leucine (L), Methionine (M), Valine (V); and (6) Phenylalanine (F), Tyrosine (Y), Tryptophan (W) (see also, Creighton, 1984, Proteins, W.H. Freeman and Company). In addition to the above-defined conservative substitutions, other modifications of amino acid residues can also result in “conservatively modified variants.” For example, one may regard all charged amino acids as substitutions for each other whether they are positive or negative. In addition, conservatively modified variants can also result from individual substitutions, deletions or additions which alter, add or delete a single amino acid or a small percentage of amino acids, for example, often less than 5%, in an encoded sequence. Further, a conservatively modified variant can be made from a recombinant polypeptide by substituting a codon for an amino acid employed by the native or wild-type gene with a different codon for the same amino acid.

The term “RNA” as used herein is defined as ribonucleic acid. The term “recombinant DNA” as used herein is defined as DNA produced by joining pieces of DNA from different sources.

The term “recombinant polypeptide” as used herein is defined as a polypeptide produced by using recombinant DNA methods.

The term “single chain antibody” is an antibody that contains an antigen binding site that is composed of a single polypeptide chain. One example of a single chain antibody is a single-chain variable fragment (scFv) antibody, which is a fusion protein that contains the variable regions of the heavy (VH) and light chains (VL) of a classical antibody connected by a short linker peptide of about ten to about 25 amino acids. A single-chain antibody can also be obtained by immunization of a camelid (e.g., a camel, llama or alpaca) or a cartilaginous fish (e.g., a shark), which make antibodies that are composed of only heavy chains. A monomeric variable domain of a heavy chain antibody binds antigen.

By “pharmaceutically acceptable” it is meant, for example, a carrier, diluent or excipient that is compatible with the other ingredients of the formulation and generally safe for administration to a recipient thereof. As used herein, “pharmaceutically acceptable carrier” includes any material, which when combined with the conjugate retains the conjugates' activity and is non-reactive with the subject's immune systems. Examples include, but are not limited to, any of the standard pharmaceutical carriers such as a phosphate buffered saline solution, water, emulsions such as oil/water emulsion, and various types of wetting agents. Other carriers may also include sterile solutions, tablets including coated tablets and capsules. Typically such carriers contain excipients such as starch, milk, sugar, certain types of clay, gelatin, stearic acid or salts thereof, magnesium or calcium stearate, talc, vegetable fats or oils, gums, glycols, or other known excipients. Such carriers may also include flavor and color additives or other ingredients. Compositions comprising such carriers are formulated by well-known conventional methods.

The term “a population of cells that comprise a library of surface-tethered extracellular capture agents” refers to a population of that cells that expresses (i.e., “displays”) a surface-tethered capture agent on their exterior surface and the amino acid sequence of the capture agent differs from cell to cell.

The terms “patient,” “subject,” “individual,” and the like are used interchangeably herein, and refer to any animal, preferably a mammal, and most preferably a human, having a complement system, including a human in need of therapy for, or susceptible to, a condition or its sequelae. Thus, the individual may include, for example, dogs, cats, pigs, cows, sheep, goats, horses, rats, monkeys, and mice and humans.

The phrase “percent (%) identity” refers to the percentage of sequence similarity found in a comparison of two or more amino acid sequences. Percent identity can be determined electronically using any suitable software. Likewise, “similarity” between two polypeptides (or one or more portions of either or both of them) is determined by comparing the amino acid sequence of one polypeptide to the amino acid sequence of a second polypeptide. Any suitable algorithm useful for such comparisons can be adapted for application in the context of the invention.

A “therapeutic” treatment is a treatment administered to a subject who exhibits signs of pathology, for the purpose of diminishing or eliminating those signs.

“Therapeutically effective amount” is an amount of a compound of the invention, that when administered to a patient, ameliorates a symptom of the disease. The amount of a compound of the invention which constitutes a “therapeutically effective amount” will vary depending on the compound, the disease state and its severity, the age of the patient to be treated, and the like. The therapeutically effective amount can be determined routinely by one of ordinary skill in the art having regard to his own knowledge and to this disclosure.

The terms “treat,” “treating,” and “treatment,” refer to therapeutic or preventative measures described herein. The methods of “treatment” employ administration to a subject, in need of such treatment, a composition of the present invention, for example, a subject afflicted a disease or disorder, or a subject who ultimately may acquire such a disease or disorder, in order to prevent, cure, delay, reduce the severity of, or ameliorate one or more symptoms of the disorder or recurring disorder, or in order to prolong the survival of a subject beyond that expected in the absence of such treatment.

“Variant” as the term is used herein, is a nucleic acid sequence or a peptide sequence that differs in sequence from a reference nucleic acid sequence or peptide sequence respectively, but retains essential biological properties of the reference molecule. Changes in the sequence of a nucleic acid variant may not alter the amino acid sequence of a peptide encoded by the reference nucleic acid, or may result in amino acid substitutions, additions, deletions, fusions and truncations. Changes in the sequence of peptide variants are typically limited or conservative, so that the sequences of the reference peptide and the variant are closely similar overall and, in many regions, identical. A variant and reference peptide can differ in amino acid sequence by one or more substitutions, additions, deletions in any combination. A variant of a nucleic acid or peptide can be a naturally occurring such as an allelic variant, or can be a variant that is not known to occur naturally. Non-naturally occurring variants of nucleic acids and peptides may be made by mutagenesis techniques or by direct synthesis

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### DESCRIPTION

This invention is based, in part, on the development of binding molecules that inhibit or block the activation of the SARS-CoV-2 Replication and Transcription Complex (RTC). In some embodiments, the invention provides antibodies or nanobodies that bind with high affinity to a SARS-CoV-2 non-structural protein (Nsp). In one embodiment, the invention provides antibodies or nanobodies that bind with high affinity to SARS-CoV-2 Nsp7, Nsp8, Nsp9, Nsp12, or Nsp13. In one embodiment, the invention provides antibodies or nanobodies that bind with high affinity to SARS-CoV-2 Nsp9. In one embodiment, the invention provides antibodies or nanobodies that promote oligomerization of at least one SARS-CoV-2 non-structural protein (Nsp). In one embodiment, the invention provides antibodies or nanobodies that promote oligomerization of Nsp9. In some embodiments, the invention relates to methods of using the binding molecules (e.g, antibodies or nanobodies) of the invention to bind to their target protein. In some embodiments, the invention relates to methods of using the binding molecules (e.g, antibodies or nanobodies) of the invention to treat or prevent viral replication. In some embodiments, the invention relates to methods of using the binding molecules (e.g, antibodies or nanobodies) of the invention to treat or prevent a diseases or disorder associated with SARS-CoV-2 (e.g., COVID-19). In various embodiments, the invention is directed to compositions and methods for treating a disease or disorder in an individual by administering to a subject in need thereof at least one binding molecule (e.g, antibody or nanobody) of the invention.

In certain embodiments, the binding molecule of the invention is considered an antibody because it binds to a target (e.g., SARS-CoV-2 Nsp). In one embodiment, the antibody comprises a heavy chain constant region, such as an IgG1, IgG2, IgG3, IgG4, IgA, IgE, IgM or IgD constant region. Preferably, the heavy chain constant region is an IgG1 heavy chain constant region or an IgG4 heavy chain constant region. Furthermore, the antibody can comprise a light chain constant region, either a kappa light chain constant region or a lambda light chain constant region. In one embodiment, the antibody comprises a kappa light chain constant region. Alternatively, the antibody portion can be, for example, a Fab fragment or a single chain Fv fragment.

In some embodiments, the compositions of the invention decrease the level or activity (e.g., enzymatic activity, substrate binding activity, etc.) of the target protein or peptide. The binding molecules of the invention include a variety of forms of antibodies including, for example, polyclonal antibodies, monoclonal antibodies, intracellular antibodies (“intrabodies”), Fv, Fab and F(ab)2, single chain antibodies (scFv), heavy chain antibodies (such as camelid antibodies), synthetic antibodies, chimeric antibodies, nanobodies and humanized antibodies.

In some embodiments, the invention provides engineered heavy chain antibodies, or nanobodies. As with other antibodies of non-human origin, an amino acid sequence of a nanobody can be altered recombinantly to obtain a sequence that more closely resembles a human sequence, i.e., the nanobody can be “humanized” to thereby further reduce the potential immunogenicity of the antibody.

In some embodiments, the invention relates to the binding portion of an antibody or nanobody that comprises one or more fragments of an antibody or nanobody that retain the ability to specifically bind to binding partner molecule. It has been shown that the binding function of an antibody can be performed by fragments of a full-length antibody. Examples of binding fragments encompassed within the term “binding portion” of an antibody include (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546), which consists of a VH domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see e.g., Bird et al. (1988) Science 242:423-426; and Huston et al. (1988) Proc. Natl. Acad. Sci. USA 85:5879-5883). Such single chain antibodies are also intended to be encompassed within the term “binding portion” of an antibody. These antibody fragments are obtained using conventional techniques known to those with skill in the art, and the fragments are screened for utility in the same manner as are intact antibodies. Binding portions can be produced by recombinant DNA techniques, or by enzymatic or chemical cleavage of intact immunoglobulins.

### Nanobody Compositions

In some embodiments, the invention provides single chain antibodies, or nanobody polypeptides, that are directed against or can specifically bind to a SARS-CoV-2 Nsp, as well as compounds and constructs, (e.g., fusion proteins and polypeptides) that comprise at least one such amino acid sequence, and nucleic acid molecules encoding the nanobodies of the invention.

It should be noted that, in general, the term nanobody as used herein is not limited to a specific biological material or a specific method of preparation. For example, methods for preparing the nanobodies of the present invention include, but are not limited to, (1) isolation of a V_{HH} domain of a natural heavy chain antibody, (2) expression of a nucleotide sequence encoding a natural Vin domain, (3) humanization of natural V_{HH} domains or expression of nucleic acids encoding the humanized V_{HH} domains, and (4) camelization of natural V_{H} domains from any animal species, particularly mammals (eg, humans), or expression of a nucleic acid encoding a camelized V_{H} domain, (5) and synthesis of nanobodies or nucleic acids encoding nanobodies using amino acid or nucleic acid synthesis techniques. Suitable methods and techniques for carrying out the above will be apparent to those skilled in the art based on the disclosure herein, including, for example, the methods and techniques detailed below.

In some embodiments, the nanobodies of the present invention comprise an amino acid sequence that matches the amino acid sequence of a natural V_{HH} domain, but is “humanized” by substitution of one or more amino acid residues of the amino acid sequence of said native V_{HH} sequence with one or more amino acid residues occurring at corresponding positions of a V_{H} domain from a conventional human 4-chain antibody. The humanized nanobody of the present invention can be obtained by any suitable method known in the art.

In some embodiments, the nanobodies of the present invention are derived from a conventional 4-chain antibody by “camelization” (ie, substitution of one or more amino acid residues of a V_{H} domain with one or more amino acid residues occurring at corresponding positions in the V_{HH} domain of the heavy chain antibody). In some embodiments, the camelization occurs at the amino acid position present at the V_{H}-V_{L} junction and so-called Camelidae characteristic residues (see eg WO 94/04678). The camelized nanobody of the present invention can be obtained by any appropriate method known in the art.

In various embodiments, the invention provides nucleic acid molecules encoding the nanobodies, including humanized or camelized nanobodies, of the invention is It can be carried out by expressing the nucleotide sequence thus obtained. In some embodiments, a nucleotide sequence encoding the humanized or camelized nanobody of interest of the present invention is designed, and the nucleic acid sequences thus obtained can be expressed in order to provide the nanobodies of interest of the present invention.

In one embodiment, the nanobodies of the invention binds to and thereby partially or substantially alters at least one biological activity of the target (e.g., enzymatic activity, substrate binding activity etc.). In one embodiment, the nanobodies of the invention binds to and promotes oligomerization of the target.

In some embodiments, a nanobody that binds to a SARS-CoV-2 Nsp inhibits, blocks, or interferes with at least one activity of SARS-CoV-2 Nsp in vitro, in situ and/or in vivo. In one embodiment, the nanobody that binds to a SARS-CoV-2 Nsp inhibits, blocks, or interferes with activation of the viral RTC complex.

In some embodiments, the invention includes compositions comprising an antibody or nanobody that specifically binds to SARS-CoV-2 Nsp9.

In one aspect, this disclosure provides a nanobody, or binding portion thereof, comprising at least one, two or all three CDR sequences of SEQ ID NO:1-SEQ ID NO:3. In one aspect, this disclosure provides a nanobody, or binding portion thereof, comprising at least one, two or all three CDR sequences of SEQ ID NO:9-SEQ ID NO:11. In one aspect, this disclosure provides a nanobody, or binding portion thereof, comprising at least one, two or all three CDR sequences of SEQ ID NO: 17-SEQ ID NO:19. In one aspect, this disclosure provides a nanobody, or binding portion thereof, comprising at least one, two or all three CDR sequences of SEQ ID NO:25-SEQ ID NO:27. In one aspect, this disclosure provides a nanobody, or binding portion thereof, comprising at least one, two or all three CDR sequences of SEQ ID NO:33-SEQ ID NO:35. In one aspect, this disclosure provides a nanobody, or binding portion thereof, comprising at least one, two or all three CDR sequences of SEQ ID NO:41-SEQ ID NO:43. In one aspect, this disclosure provides a nanobody, or binding portion thereof, comprising at least one, two or all three CDR sequences of SEQ ID NO:49-SEQ ID NO:51. In one aspect, this disclosure provides a nanobody, or binding portion thereof, comprising at least one, two or all three CDR sequences of SEQ ID NO:57-SEQ ID NO:59. In one aspect, this disclosure provides a nanobody, or binding portion thereof, comprising at least one, two or all three CDR sequences of SEQ ID NO:65-SEQ ID NO:67. In one aspect, this disclosure provides a nanobody, or binding portion thereof, comprising at least one, two or all three CDR sequences of SEQ ID NO:73-SEQ ID NO:75. In one aspect, this disclosure provides a nanobody, or binding portion thereof, comprising at least one, two or all three CDR sequences of SEQ ID NO:81-SEQ ID NO:83. In one aspect, this disclosure provides a nanobody, or binding portion thereof, comprising at least one, two or all three CDR sequences of SEQ ID NO:89-SEQ ID NO:91.

In one aspect, this disclosure provides a nucleic acid molecule comprising a nucleotide sequence encoding a nanobody, or binding portion thereof, comprising at least one, two or all three CDR sequences of SEQ ID NO:1-SEQ ID NO:3. Accordingly, in some embodiments, the invention comprises a nucleic acid molecule comprising a nucleotide sequence comprising one, two or all three CDR sequences of SEQ ID NO:5-SEQ ID NO:7.

In one aspect, this disclosure provides a nucleic acid molecule comprising a nucleotide sequence encoding a nanobody, or binding portion thereof, comprising at least one, two or all three CDR sequences of SEQ ID NO:9-SEQ ID NO:11. Accordingly, in some embodiments, the invention comprises a nucleic acid molecule comprising a nucleotide sequence comprising one, two or all three CDR sequences of SEQ ID NO: 13-SEQ ID NO:15.

In one aspect, this disclosure provides a nucleic acid molecule comprising a nucleotide sequence encoding a nanobody, or binding portion thereof, comprising at least one, two or all three CDR sequences of SEQ ID NO:17-SEQ ID NO:19. Accordingly, in some embodiments, the invention comprises a nucleic acid molecule comprising a nucleotide sequence comprising one, two or all three CDR sequences of SEQ ID NO:21-SEQ ID NO:23.

In one aspect, this disclosure provides a nucleic acid molecule comprising a nucleotide sequence encoding a nanobody, or binding portion thereof, comprising at least one, two or all three CDR sequences of SEQ ID NO:25-SEQ ID NO:27. Accordingly, in some embodiments, the invention comprises a nucleic acid molecule comprising a nucleotide sequence comprising one, two or all three CDR sequences of SEQ ID NO:29-SEQ ID NO:31.

In one aspect, this disclosure provides a nucleic acid molecule comprising a nucleotide sequence encoding a nanobody, or binding portion thereof, comprising at least one, two or all three CDR sequences of SEQ ID NO:33-SEQ ID NO:35. Accordingly, in some embodiments, the invention comprises a nucleic acid molecule comprising a nucleotide sequence comprising one, two or all three CDR sequences of SEQ ID NO:37-SEQ ID NO:39.

In one aspect, this disclosure provides a nucleic acid molecule comprising a nucleotide sequence encoding a nanobody, or binding portion thereof, comprising at least one, two or all three CDR sequences of SEQ ID NO:41-SEQ ID NO:43. Accordingly, in some embodiments, the invention comprises a nucleic acid molecule comprising a nucleotide sequence comprising one, two or all three CDR sequences of SEQ ID NO:45-SEQ ID NO:47.

In one aspect, this disclosure provides a nucleic acid molecule comprising a nucleotide sequence encoding a nanobody, or binding portion thereof, comprising at least one, two or all three CDR sequences of SEQ ID NO:49-SEQ ID NO:51. Accordingly, in some embodiments, the invention comprises a nucleic acid molecule comprising a nucleotide sequence comprising one, two or all three CDR sequences of SEQ ID NO:53-SEQ ID NO:55.

In one aspect, this disclosure provides a nucleic acid molecule comprising a nucleotide sequence encoding a nanobody, or binding portion thereof, comprising at least one, two or all three CDR sequences of SEQ ID NO:57-SEQ ID NO:59. Accordingly, in some embodiments, the invention comprises a nucleic acid molecule comprising a nucleotide sequence comprising one, two or all three CDR sequences of SEQ ID NO:61-SEQ ID NO:63.

In one aspect, this disclosure provides a nucleic acid molecule comprising a nucleotide sequence encoding a nanobody, or binding portion thereof, comprising at least one, two or all three CDR sequences of SEQ ID NO:65-SEQ ID NO:67. Accordingly, in some embodiments, the invention comprises a nucleic acid molecule comprising a nucleotide sequence comprising one, two or all three CDR sequences of SEQ ID NO:69-SEQ ID NO:71.

In one aspect, this disclosure provides a nucleic acid molecule comprising a nucleotide sequence encoding a nanobody, or binding portion thereof, comprising at least one, two or all three CDR sequences of SEQ ID NO:73-SEQ ID NO:75. Accordingly, in some embodiments, the invention comprises a nucleic acid molecule comprising a nucleotide sequence comprising one, two or all three CDR sequences of SEQ ID NO:77-SEQ ID NO:79.

In one aspect, this disclosure provides a nucleic acid molecule comprising a nucleotide sequence encoding a nanobody, or binding portion thereof, comprising at least one, two or all three CDR sequences of SEQ ID NO:81-SEQ ID NO:83. Accordingly, in some embodiments, the invention comprises a nucleic acid molecule comprising a nucleotide sequence comprising one, two or all three CDR sequences of SEQ ID NO:85-SEQ ID NO:87.

In one aspect, this disclosure provides a nucleic acid molecule comprising a nucleotide sequence encoding a nanobody, or binding portion thereof, comprising at least one, two or all three CDR sequences of SEQ ID NO:89-SEQ ID NO:91. Accordingly, in some embodiments, the invention comprises a nucleic acid molecule comprising a nucleotide sequence comprising one, two or all three CDR sequences of SEQ ID NO:93-SEQ ID NO:95.

The antibody CDR sequences provided establish a novel family of binding molecules, in accordance with this invention, comprising polypeptides that include the CDR sequences listed.

In some embodiments, the invention comprises a nanobody comprising an amino acid sequence of SEQ ID NO:4, SEQ ID NO: 12, SEQ ID NO:20, SEQ ID NO:28, SEQ ID NO:36, SEQ ID NO:44, SEQ ID NO:52, SEQ ID NO:60, SEQ ID NO:68, SEQ ID NO:76, SEQ ID NO:84 or SEQ ID NO:92. In some embodiments, the invention comprises a nucleic acid molecule comprising a nucleotide sequence encoding a nanobody comprising an amino acid sequence of SEQ ID NO:4, SEQ ID NO: 12, SEQ ID NO:20, SEQ ID NO:28, SEQ ID NO:36, SEQ ID NO:44, SEQ ID NO:52, SEQ ID NO:60, SEQ ID NO:68, SEQ ID NO:76, SEQ ID NO:84 or SEQ ID NO:92. In some embodiments, the invention comprises a nucleic acid molecule comprising a nucleotide sequence of SEQ ID NO:8, SEQ ID NO: 16, SEQ ID NO:24, SEQ ID NO:32, SEQ ID NO:40, SEQ ID NO:48, SEQ ID NO:56, SEQ ID NO:64, SEQ ID NO: 72, SEQ ID NO:80, SEQ ID NO:88 or SEQ ID NO:96. In one embodiment the binding molecules of the invention have specific binding and/or detection and/or inhibitory activity. In one embodiment, standard methods known in the art for generating binding proteins of the present invention and assessing the binding and/or detection and/or inhibitory characteristics of those binding protein may be used to identify binding molecules of the invention with an increased or desired level of binding to a target. In some embodiments, the target is SARS-CoV-2 Nsp9. In some embodiments, binding molecules with an increased or desired level of binding to SARS-CoV-2 Nsp9 comprises an amino acid sequence of SEQ ID NO:4, SEQ ID NO: 12, SEQ ID NO:20, SEQ ID NO:28, SEQ ID NO:36, SEQ ID NO:44, SEQ ID NO:52, or SEQ ID NO:60. In some embodiments, binding molecules with an increased or desired level of binding to Nsp9 are encoded by a nucleic acid molecule comprising a nucleotide sequence of SEQ ID NO:8, SEQ ID NO:16, SEQ ID NO:24, SEQ ID NO:32, SEQ ID NO:40, SEQ ID NO:48, SEQ ID NO:56, or SEQ ID NO:64.

In some embodiments, the binding molecules (e.g., nanobodies) of the present invention, are operably linked or fused to an additional amino acid sequence. For example, in some embodiments, the nanobody sequence can also include additional sequences that encode linker, leader, or tag sequences that are fused or linked to the nanobody of the invention by a peptide bond. In some embodiments, the molecules described herein may contain a tag or detectable moiety. This tag or detectable moiety can be fused to the C-terminus or N-terminus of the protein, peptide, antibody, antibody fragment, or fusion molecule of the invention. In some embodiments, the tag or detectable moiety can be used to facilitate protein purification. In some embodiments, the tag or detectable moiety allows for visualization of the molecule using various imaging modalities.

In some embodiments, the binding molecules (e.g., nanobodies) of the present invention, are operably linked or fused to a tag (e.g., FLAG, polyhistidine (His), hemagglutinin (HA), glutathione-S-transferase (GST), or maltose-binding protein (MBP)) for use in purifying the antibodies. In embodiments, the binding molecules (e.g., nanobodies) of the present invention, are operably linked or fused to a diagnostic or detectable marker, for example, luciferase, green fluorescent protein (GFP), or chloramphenicol acetyl transferase (CAT).

In one embodiment, the nanobodies comprise a 6×His tag linked to the nanobody by a linker sequence. Exemplary nanobody sequences comprising a linker and tag are set forth in SEQ ID NO: 105, SEQ ID NO: 107, SEQ ID NO: 109, SEQ ID NO: 111, SEQ ID NO:113, SEQ ID NO:115, SEQ ID NO: 117, SEQ ID NO: 119, SEQ ID NO:121, SEQ ID NO:123, SEQ ID NO:125, and SEQ ID NO:127. Exemplary nucleotide sequence encoding nanobody sequences comprising a linker and tag are set forth in SEQ ID NO:106, SEQ ID NO:108, SEQ ID NO: 110, SEQ ID NO: 112, SEQ ID NO: 114, SEQ ID NO: 116, SEQ ID NO:118, SEQ ID NO:120, SEQ ID NO: 122, SEQ ID NO: 124, SEQ ID NO: 126, and SEQ ID NO:128.

In some embodiments, the binding molecules (e.g., nanobodies) of the present invention, exhibit a high capacity to detect and bind their target, (e.g., SARS-CoV-2 Nsp9), in a complex mixture of salts, compounds and other polypeptides, e.g., as assessed by any one of several in vitro and in vivo assays known in the art. The skilled artisan will understand that the binding molecules (e.g., nanobodies, etc.) described herein as useful in the methods of diagnosis and treatment and prevention of disease, are also useful in procedures and methods of the invention that include, but are not limited to, an immunochromatography assay, an immunodot assay, a Luminex assay, an ELISA assay, an ELISPOT assay, a protein microarray assay, a Western blot assay, a mass spectrophotometry assay, a radioimmunoassay (RIA), a radioimmunodiffusion assay, a liquid chromatography-tandem mass spectrometry assay, an ouchterlony immunodiffusion assay, reverse phase protein microarray, a rocket immunoelectrophoresis assay, an immunohistostaining assay, an immunoprecipitation assay, a complement fixation assay, FACS, a protein chip assay, separation and purification processes, and affinity chromatography (see also, 2007, Van Emon, Immunoassay and Other Bioanalytical Techniques, CRC Press; 2005, Wild, Immunoassay Handbook, Gulf Professional Publishing; 1996, Diamandis and Christopoulos, Immunoassay, Academic Press; 2005, Joos, Microarrays in Clinical Diagnosis, Humana Press; 2005, Hamdan and Righetti, Proteomics Today, John Wiley and Sons; 2007).

In some embodiments, the binding molecules (e.g., nanobodies, etc.) of the present invention, exhibit a high capacity to reduce or to inhibit an activity of their target (e.g., enzymatic activity, substrate binding activity, etc.) as assessed by any one of several in vitro and in vivo assays known in the art. In some embodiments, the binding molecule (e.g., nanobody, etc.) binds to its target protein with a K_{D} of 1×10⁻⁶ M or less, 1×10⁻⁷ M or less, 1×10⁻⁸ M or less, 5×10⁻⁹ M or less, 1×10⁻⁹ M or less, or 3×10⁻¹⁰ M or less. The term “does not substantially bind” to a protein or cells, as used herein, means does not bind or does not bind with a high affinity to the protein or cells, i.e., binds to the protein or cells with a K_{D} of greater than 1×10⁶ M or more, 1×10⁵ M or more, 1×10⁴ M or more, 1×10³ M or more, or 1×10² M or more. The term “K_{D}”, as used herein, is intended to refer to the dissociation constant, which is obtained from the ratio of Kd to Ka (i.e., Kd/Ka) and is expressed as a molar concentration (M). K_{D} values for a binding molecule (e.g., nanobody, etc.) can be determined using methods well established in the art.

### Multi-Paratopic Antibodies

In some embodiments, the present invention provides amino acid sequences and antibody compositions that are capable of binding to two or more different antigenic determinants, or epitopes. In this context, the amino acid sequences and polypeptides of the invention are also referred to as “multi-paratopic” (such as e.g. “bi-paratopic” or “tri-paratopic”, etc.) amino acid sequences and polypeptides. The multi-paratopic amino acid sequences and polypeptides of the invention can be directed against any antigenic determinants, or epitopes. For example, and generally, a bi-paratopic polypeptide of the invention may comprise at least one amino acid sequence or nanobody directed against a first antigenic determinant or epitope, and at least one amino acid sequence or nanobody directed against a second antigenic determinant or epitope different from the first antigenic determinant or epitope. In some embodiments, the amino acid sequences and/or nanobodies are linked, for example via a suitable linker.

A tri-paratopic polypeptide of the invention may comprise at least one further amino acid sequence or nanobody of the invention directed against a third antigenic determinant or epitope, different from both the first and second antigenic determinant, epitope, part or domain.

In some embodiments, multi-paratopic polypeptides of the invention may contain at least two amino acid sequences or nanobodies of the invention directed against at least two different antigenic determinants or epitopes of the same protein or peptide (e.g., two different antigenic determinants of SARS-CoV-2 Nsp9.) In some embodiments, multi-paratopic polypeptides of the invention may contain at least two amino acid sequences or nanobodies directed against at least two different antigenic determinants or epitopes of at least two different proteins or peptides (e.g., one nanobody of the invention directed against SARS-CoV-2 Nsp9 and a second nanobody directed against a different target protein.)

### Nanoparticle Formulations

In one embodiment, the nanobodies of the invention or nucleic acid molecules encoding the same may be formulated for delivery using a nanoparticle formulation. Therefore, in some embodiments, the composition of the invention may comprise a nanoparticle, including but not limited to a lipid nanoparticle (LNP), comprising a SARS-CoV-2 nanobody of the invention, or a LNP comprising a nucleic acid encoding a SARS-CoV-2 nanobody of the invention. In some embodiments, the composition comprises or encodes all or part of a SARS-CoV-2 Nsp binding molecule of the invention, or an immunogenically functional equivalent thereof. In some embodiments, the composition comprises an mRNA molecule that encodes all or part of a SARS-CoV-2 Nsp binding molecule of the invention. In one embodiment, the invention relates to a lipid-nanoparticle formulation comprising an mRNA molecule encoding SEQ ID NO:4, SEQ ID NO:12, SEQ ID NO:20, SEQ ID NO:28, SEQ ID NO:36, SEQ ID NO:44, SEQ ID NO:52, SEQ ID NO:60, SEQ ID NO:68, SEQ ID NO: 76, SEQ ID NO:84, SEQ ID NO:92, or any combination thereof. In some embodiments, the LNP comprises an mRNA molecule comprising a sequence corresponding to SEQ ID NO:8, SEQ ID NO: 16, SEQ ID NO:24, SEQ ID NO:32, SEQ ID NO:40, SEQ ID NO:48, SEQ ID NO:56, SEQ ID NO:64, SEQ ID NO:72, SEQ ID NO:80, SEQ ID NO:88, SEQ ID NO:96, or any combination thereof. For example, in one embodiment, the invention relates to a lipid-nanoparticle formulation comprising an mRNA molecule encoding the 2NSP23 nanobody comprising a sequence as set forth in SEQ ID NO:28. In some embodiments, the LNP comprises an mRNA molecule comprising a sequence corresponding to SEQ ID NO:32.

The term “lipid nanoparticle” refers to a particle having at least one dimension on the order of nanometers (e.g., 1-1,000 nm) which includes one or more lipids, for example a lipid of Formula (I)-(XV).

In various embodiments, the lipid nanoparticles have a mean diameter of from about 30 nm to about 150 nm, from about 40 nm to about 150 nm, from about 50 nm to about 150 nm, from about 60 nm to about 130 nm, from about 70 nm to about 110 nm, from about 70 nm to about 100 nm, from about 80 nm to about 100 nm, from about 90 nm to about 100 nm, from about 70 to about 90 nm, from about 80 nm to about 90 nm, from about 70 nm to about 80 nm, or about 30 nm, 35 nm, 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, or 150 nm.

In various embodiments, the lipids or the LNP are substantially non-toxic.

In various embodiments, the lipids or the LNPs described herein readily transport to a tissue of interest. For example, in various embodiments, the lipids or the LNPs described herein readily transport through a cell membrane to a cell. In various embodiments, the lipids or the LNP described herein efficiently transport through a cell membrane to a cell. In some embodiments, the lipids or the LNP described herein transport through a cell membrane to a cell with enhanced efficacy.

In one embodiment, the lipid nanoparticle comprises two or more of an ionizable cationic lipid, a neutral amphoteric or zwitterionic lipid, a non-ionic lipid, cholesterol, and a quaternary ammonium cationic lipid. In one embodiment, the lipid nanoparticle comprises three or more of an ionizable cationic lipid, a neutral amphoteric or zwitterionic lipid, a non-ionic lipid, cholesterol, and a quaternary ammonium cationic lipid. In one embodiment, the lipid nanoparticle comprises four or more of an ionizable cationic lipid, a neutral amphoteric or zwitterionic lipid, a non-ionic lipid, a steroid, and a quaternary ammonium cationic lipid. In one embodiment, the lipid nanoparticle comprises an ionizable cationic lipid, a neutral amphoteric or zwitterionic lipid, a non-ionic lipid, cholesterol, and a quaternary ammonium cationic lipid.

As used herein, the term “cationic lipid” refers to a lipid that is cationic or becomes cationic (protonated) as the pH is lowered below the pK of the ionizable group of the lipid, but is progressively more neutral at higher pH values. At pH values below the pK, the lipid is then able to associate with negatively charged nucleic acids. In certain embodiments, the cationic lipid comprises a zwitterionic lipid that assumes a positive charge on pH decrease.

In some embodiments, the cationic lipid comprises any of a number of lipid species which carry a net positive charge at a selective pH, such as physiological pH. Such lipids include, but are not limited to, N,N-dioleyl-N,N-dimethylammonium chloride (DODAC); N-(2,3-dioleyloxy)propyl)-N,N,N-trimethylammonium chloride (DOTMA); N,N-distearyl-N,N-dimethylammonium bromide (DDAB); N-(2,3-dioleoyloxy)propyl)-N,N,N-trimethylammonium chloride (DOTAP); 3-(N—(N′,N′-dimethylaminoethane)-carbamoyl)cholesterol (DC-Chol), N-(1-(2,3-dioleoyloxy)propyl)-N-2-(sperminecarboxamido)ethyl)-N,N-dimethylammonium trifluoracetate (DOSPA), dioctadecylamidoglycyl carboxyspermine (DOGS), 1,2-dioleoyl-3-dimethylammonium propane (DODAP), N,N-dimethyl-2,3-dioleoyloxy)propylamine (DODMA), and N-(1,2-dimyristyloxyprop-3-yl)-N,N-dimethyl-N-hydroxyethyl ammonium bromide (DMRIE). Additionally, a number of commercial preparations of cationic lipids are available which can be used in the present invention. These include, for example, LIPOFECTIN® (commercially available cationic liposomes comprising DOTMA and 1,2-dioleoyl-sn-3-phosphoethanolamine (DOPE), from GIBCO/BRL, Grand Island, N.Y.); LIPOFECTAMINE® (commercially available cationic liposomes comprising N-(1-(2,3-dioleyloxy)propyl)-N-(2-(sperminecarboxamido)ethyl)-N,N-dimethylammonium trifluoroacetate (DOSPA) and (DOPE), from GIBCO/BRL); and TRANSFECTAM® (commercially available cationic lipids comprising dioctadecylamidoglycyl carboxyspermine (DOGS) in ethanol from Promega Corp., Madison, Wis.). The following lipids are cationic and have a positive charge at below physiological pH: DODAP, DODMA, DMDMA, 1,2-dilinoleyloxy-N,N-dimethylaminopropane (DLinDMA), 1,2-dilinolenyloxy-N,N-dimethylaminopropane (DLenDMA).

In one embodiment, the cationic lipid is an amino lipid. Suitable amino lipids useful in the invention include those described in WO 2012/016184, incorporated herein by reference in its entirety. Representative amino lipids include, but are not limited to, 1,2-dilinoleyoxy-3-(dimethylamino)acetoxypropane (DLin-DAC), 1,2-dilinoleyoxy-3-morpholinopropane (DLin-MA), 1,2-dilinoleoyl-3-dimethylaminopropane (DLinDAP), 1,2-dilinoleylthio-3-dimethylaminopropane (DLin-S-DMA), 1-linoleoyl-2-linoleyloxy-3-dimethylaminopropane (DLin-2-DMAP), 1,2-dilinoleyloxy-3-trimethylaminopropane chloride salt (DLin-TMA.Cl), 1,2-dilinoleoyl-3-trimethylaminopropane chloride salt (DLin-TAP.Cl), 1,2-dilinoleyloxy-3-(N-methylpiperazino)propane (DLin-MPZ), 3-(N,N-dilinoleylamino)-1,2-propanediol (DLinAP), 3-(N,N-dioleylamino)-1,2-propanediol (DOAP), 1,2-dilinoleyloxo-3-(2-N,N-dimethylamino)ethoxypropane (DLin-EG-DMA), and 2,2-dilinoleyl-4-dimethylaminomethyl-[1,3]-dioxolane (DLin-K-DMA). In one embodiment, the cationic lipid is C12-200 (Corden Pharma).

The term “neutral lipid” refers to any one of a number of lipid species that exist in either an uncharged or neutral zwitterionic form at physiological pH. Representative neutral lipids include diacylphosphatidylcholines, diacylphosphatidylethanolamines, ceramides, sphingomyelins, dihydro sphingomyelins, cephalins, and cerebrosides.

Exemplary neutral lipids include, for example, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoyl-phosphatidylethanolamine (DOPE), palmitoyloleoylphosphatidylcholine (POPC), palmitoyloleoyl-phosphatidylethanolamine (POPE) and dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidylethanolamine (DSPE), distearoyl-phosphatidylethanolamine (DSPE)-maleimide-PEG, distearoyl-phosphatidylethanolamine (DSPE)-maleimide-PEG2000, 16-O-monomethyl PE, 16-O-dimethyl PE, 18-1-trans PE, 1-stearioyl-2-oleoyl-phosphatidyethanol amine (SOPE), stearoyloleoylphosphatidylcholine (SOPC), and 1,2-dielaidoyl-sn-glycero-3-phophoethanolamine (transDOPE). In one embodiment, the neutral lipid is 1,2-distearoyl-sn-glycero-3-phosphocholine (DSPC).

In some embodiments, the composition comprises a neutral lipid selected from DSPC, DPPC, DMPC, DOPC, POPC, DOPE, and SM.

A “steroid” is a compound comprising the following carbon skeleton:

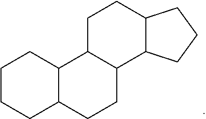 [SMILES: C1CCC2C(C1)CCC1C3CCCC3CCC21]

In certain embodiments, the steroid or steroid analogue is cholesterol. In some of these embodiments, the molar ratio of the cationic lipid.

The term “anionic lipid” refers to any lipid that is negatively charged at physiological pH. These lipids include phosphatidylglycerol, cardiolipin, diacylphosphatidylserine, diacylphosphatidic acid, N-dodecanoylphosphatidylethanolamines, N-succinylphosphatidylethanolamines, N-glutarylphosphatidylethanolamines, lysylphosphatidylglycerols, palmitoyloleyolphosphatidylglycerol (POPG), and other anionic modifying groups joined to neutral lipids.

The term “polymer conjugated lipid” refers to a molecule comprising both a lipid portion and a polymer portion. An example of a polymer conjugated lipid is a pegylated lipid. The term “pegylated lipid” refers to a molecule comprising both a lipid portion and a polyethylene glycol portion. Pegylated lipids are known in the art and include 1-(monomethoxy-polyethyleneglycol)-2,3-dimyristoylglycerol (PEG-s-DMG) and the like.

Suitable polyethylene glycol-lipids include PEG-modified phosphatidylethanolamine, PEG-modified phosphatidic acid, PEG-modified ceramides (e.g., PEG-CerC14 or PEG-CerC20), PEG-modified dialkylamines, PEG-modified diacylglycerols, PEG-modified dialkylglycerols. Representative polyethylene glycol-lipids include PEG-c-DOMG, PEG-c-DMA, and PEG-s-DMG. In one embodiment, the polyethylene glycol-lipid is N-[(methoxy poly(ethylene glycol)₂₀₀₀)carbamyl]-1,2-dimyristyloxlpropyl-3-amine (PEG-c-DMA). In one embodiment, the polyethylene glycol-lipid is PEG-c-DOMG). In other embodiments, the LNPs comprise a pegylated diacylglycerol (PEG-DAG) such as 1-(monomethoxy-polyethyleneglycol)-2,3-dimyristoylglycerol (PEG-DMG), a pegylated phosphatidylethanoloamine (PEG-PE), a PEG succinate diacylglycerol (PEG-S-DAG) such as 4-O-(2′,3′-di(tetradecanoyloxy)propyl-1-O-(ω-methoxy(polyethoxy)ethyl)butanedioate (PEG-S-DMG), a pegylated ceramide (PEG-cer), or a PEG dialkoxypropylcarbamate such as ω-methoxy(polyethoxy)ethyl-N-(2,3-di(tetradecanoxy)propyl)carbamate or 2,3-di(tetradecanoxy)propyl-N-(ω-methoxy(polyethoxy)ethyl)carbamate.

In various embodiments, the LNP comprises one or more lipids in a concentration range of about 0.1 mol % to about 100 mol %. In some embodiments, the LNP comprises one or more lipids in a concentration range of about 1 mol % to about 100 mol %. In some embodiments, the LNP comprises one or more lipids in a concentration range of about 10 mol % to about 70 mol %. In some embodiments, the LNP comprises one or more lipids in a concentration range of about 10 mol % to about 50 mol %. In some embodiments, the LNP comprises one or more lipids in a concentration range of about 15 mol % to about 45 mol %. In some embodiments, the LNP comprises one or more lipids in a concentration range of about 35 mol % to about 40 mol %.

For example, in some embodiments, the LNP comprises one or more lipids in a concentration of about 1 mol %. In some embodiments, the LNP comprises one or more lipids in a concentration of about 2 mol %. In some embodiments, the LNP comprises one or more lipids in a concentration of about 5 mol %. In some embodiments, the LNP comprises one or more lipids in a concentration of about 5.5 mol %. In some embodiments, the LNP comprises one or more lipids in a concentration of about 10 mol %. In some embodiments, the LNP comprises one or more lipids in a concentration of about 12 mol %. In some embodiments, the LNP comprises one or more lipids in a concentration of about 15 mol %. In some embodiments, the LNP comprises one or more lipids in a concentration of about 20 mol %. In some embodiments, the LNP comprises one or more lipids in a concentration of about 25 mol %. In some embodiments, the LNP comprises one or more lipids in a concentration of about 30 mol %. In some embodiments, the LNP comprises one or more lipids in a concentration of about 35 mol %. In some embodiments, the LNP comprises one or more lipids in a concentration of about 37 mol %. In some embodiments, the LNP comprises one or more lipids in a concentration of about 40 mol %. In some embodiments, the LNP comprises one or more lipids in a concentration of about 45 mol %. In some embodiments, the LNP comprises one or more lipids in a concentration of about 50 mol %. In some embodiments, the LNP comprises one or more lipids in a concentration of about 60 mol %. In some embodiments, the LNP comprises one or more lipids in a concentration of about 70 mol %. In some embodiments, the LNP comprises one or more lipids in a concentration of about 80 mol %. In some embodiments, the LNP comprises one or more lipids in a concentration of about 90 mol %. In some embodiments, the LNP comprises one or more lipids in a concentration of about 95 mol %. In some embodiments, the LNP comprises one or more lipids in a concentration of about 95.5 mol %. In some embodiments, the LNP comprises one or more lipids in a concentration of about 99 mol %. In some embodiments, the LNP comprises one or more lipids in a concentration of about 99.9 mol %. In some embodiments, the LNP comprises one or more lipids in a concentration of about 100 mol %.

In various embodiments, the LNP further comprises at least one helper compound. In some embodiments, the helper compound is a helper lipid, helper polymer, or any combination thereof. In some embodiments, the helper lipid is phospholipid, cholesterol lipid, polymer, cationic lipid, neutral lipid, charged lipid, steroid, steroid analogue, polymer conjugated lipid, stabilizing lipid, or any combination thereof.

In various embodiments, the LNP comprises one or more helper compound in a concentration range of about 0 mol % to about 100 mol %. In some embodiments, the LNP comprises one or more helper compound in a concentration range of about 0.01 mol % to about 99.9 mol %. In some embodiments, the LNP comprises one or more helper compound in a concentration range of about 0.1 mol % to about 90 mol %. In some embodiments, the LNP comprises one or more helper compound in a concentration range of about 0.1 mol % to about 70 mol %. In some embodiments, the LNP comprises one or more helper compound in a concentration range of about 5 mol % to about 95 mol %. In some embodiments, the LNP comprises one or more helper compound in a concentration range of about 0.5 mol % to about 50 mol %. In some embodiments, the LNP comprises one or more helper compound in a concentration range of about 0.5 mol % to about 47 mol %. In some embodiments, the LNP comprises one or more helper compound in a concentration range of about 2.5 mol % to about 47 mol %.

For example, in some embodiments, the LNP comprises one or more helper compound in a concentration of about 0.01 mol %. In some embodiments, the LNP comprises one or more helper compound in a concentration of about 0.1 mol %. In some embodiments, the LNP comprises one or more helper compound in a concentration of about 0.5 mol %. In some embodiments, the LNP comprises one or more helper compound in a concentration of about 1 mol %. In some embodiments, the LNP comprises one or more helper compound in a concentration of about 1.5 mol %. In some embodiments, the LNP comprises one or more helper compound in a concentration of about 2 mol %. In some embodiments, the LNP comprises one or more helper compound in a concentration of about 2.5 mol %. In some embodiments, the LNP comprises one or more helper compound in a concentration of about 5 mol %. In some embodiments, the LNP comprises one or more helper compound in a concentration of about 10 mol %. In some embodiments, the LNP comprises one or more helper compound in a concentration of about 12 mol %. In some embodiments, the LNP comprises one or more helper compound in a concentration of about 15 mol %. In some embodiments, the LNP comprises one or more helper compound in a concentration of about 16 mol %. In some embodiments, the LNP comprises one or more helper compound in a concentration of about 20 mol %. In some embodiments, the LNP comprises one or more helper compound in a concentration of about 25 mol %. In some embodiments, the LNP comprises one or more helper compound in a concentration of about 30 mol %. In some embodiments, the LNP comprises one or more helper compound in a concentration of about 35 mol %. In some embodiments, the LNP comprises one or more helper compound in a concentration of about 37 mol %. In some embodiments, the LNP comprises one or more helper compound in a concentration of about 40 mol %. In some embodiments, the LNP comprises one or more helper compound in a concentration of about 45 mol %. In some embodiments, the LNP comprises one or more helper compound in a concentration of about 46.5 mol %. In some embodiments, the LNP comprises one or more helper compound in a concentration of about 47 mol %. In some embodiments, the LNP comprises one or more helper compound in a concentration of about 50 mol %. In some embodiments, the LNP comprises one or more helper compound in a concentration of about 60 mol %. In some embodiments, the LNP comprises one or more helper compound in a concentration of about 63 mol %. In some embodiments, the LNP comprises one or more helper compound in a concentration of about 70 mol %. In some embodiments, the LNP comprises one or more helper compound in a concentration of about 80 mol %. In some embodiments, the LNP comprises one or more helper compound in a concentration of about 90 mol %. In some embodiments, the LNP comprises one or more helper compound in a concentration of about 95 mol %. In some embodiments, the LNP comprises one or more helper compound in a concentration of about 95.5 mol %. In some embodiments, the LNP comprises one or more helper compound in a concentration of about 99 mol %. In some embodiments, the LNP comprises one or more helper compound in a concentration of about 100 mol %.

In some embodiments, the phospholipid is dioleoyl-phosphatidylethanolamine (DOPE) or a derivative thereof, distearoylphosphatidylcholine (DSPC) or a derivative thereof, distearoyl-phosphatidylethanolamine (DSPE) or a derivative thereof, stearoyloleoylphosphatidylcholine (SOPC) or a derivative thereof, 1-stearioyl-2-oleoyl-phosphatidyethanol amine (SOPE) or a derivative thereof, N-(2,3-dioleoyloxy)propyl)-N,N,N-trimethylammonium chloride (DOTAP) or a derivative thereof, or any combination thereof.

In some embodiments, the LNP comprises a cationic lipid in a concentration range of about 0 mol % to about 100 mol %. In some embodiments, the LNP comprises a cationic lipid in a concentration range of about 20 mol % to about 50 mol %. In some embodiments, the LNP comprises a cationic lipid in a concentration range of about 25 mol % to about 35 mol %. In some embodiments, the LNP comprises a cationic lipid in a concentration of about 29.8 mol %.

In some embodiments, the LNP comprises a phospholipid in a concentration range of about 0 mol % to about 100 mol %. In some embodiments, the LNP comprises a phospholipid in a concentration range of about 5 mol % to about 50 mol %. In some embodiments, the LNP comprises a phospholipid in a concentration range of about 5 mol % to about 30 mol %. In some embodiments, the LNP comprises a phospholipid in a concentration range of about 10 mol % to about 15 mol %. In some embodiments, the LNP comprises a phospholipid in a concentration of about 13.6 mol %.

In some embodiments, the steroid is cholesterol or a derivative thereof. In some embodiments, the LNP comprises cholesterol in a concentration range of about 0 mol % to about 100 mol %. In some embodiments, the LNP comprises cholesterol in a concentration range of about 20 mol % to about 50 mol %. In some embodiments, the LNP comprises cholesterol in a concentration range of about 35 mol % to about 45 mol %. In some embodiments, the LNP comprises cholesterol in a concentration of about 39.5 mol %.

In some embodiments, the LNP comprises a polymer such as polyethylene glycol (PEG) or a derivative thereof. For example, in some embodiments, the LNP comprises a polymer in a concentration range of about 0 mol % to about 100 mol %. In some embodiments, the LNP comprises a polymer in a concentration range of about 0.5 mol % to about 10 mol %. In some embodiments, the LNP comprises a polymer in a concentration range of about 0.5 mol % to about 3.5 mol %. In some embodiments, the LNP comprises cholesterol in a concentration of about 2.1 mol %.

In certain embodiments, the LNP comprises a quaternary ammonium cationic lipid. In certain embodiments, the quaternary ammonium cationic lipid is present in the LNP in an amount from about 1 mol % to about 20 mol %. In one embodiment, the quaternary ammonium cationic lipid is present in the LNP in an amount from about 10 mol % to about 20 mol %. In one embodiment, the quaternary ammonium cationic lipid is present in the LNP in about 15 mol %.

In one embodiment, the lipid nanoparticle comprises a mixture of C12-200 (Corden Pharma), 1,2-dioleoyl-sn-glycero-3-phosphoethanolamine (DOPE), Cholesterol, PEGylated myristoyl diglyceride (DMG-PEG) and 1,2-di-(9Z-octadecenoyl)-3-trimethylammonium propane methylsulfate (DOTAP) in a molar ratio of 29.8:13.6:39.5:2.1:15.

In one embodiment, the composition further comprises one or more additional agents. Additional agents include, but are not limited to, one or more additional lipid or one or more additional PEG molecule, an additional antigen or antigen binding molecule, a targeting molecule, an immunomodulator, or an adjuvant.

### Methods of Using the Binding Molecules

Given the properties of the protein binding molecules (e.g., nanobodies, etc.) of the present invention, the protein binding molecules are suitable as diagnostic, therapeutic and prophylactic agents for diagnosing, treating or preventing diseases or disorders associated with SARS-CoV-2 infection in humans and animals.

In general, use comprises administering a therapeutically or prophylactically effective amount of one or more nanobodies or binding fragments of the present invention to a subject diagnosed with, or at risk of, SARS-CoV-2 infection. Any active form of the binding molecules of the invention (e.g., nanobodies, etc.) can be administered, including antibody Fab and F(ab′)2 fragments.

Treatment of individuals may comprise the administration of a therapeutically effective amount of the binding molecule of the present invention. The binding molecule can be provided in a kit as described below. The binding molecule can be used or administered as a mixture, for example in equal amounts, or individually, provided in sequence, or administered all at once. In providing a patient with the binding molecule, the dosage of administered agent will vary depending upon such factors as the patient's age, weight, height, sex, general medical condition, previous medical history, etc.

In general, if administering a systemic dose of a binding molecule, it is desirable to provide the recipient with a dosage of a binding molecule which is in the range of from about 1 ng/kg-100 ng/kg, 100 ng/kg-500 ng/kg, 500 ng/kg-1 μg/kg, 1 μg/kg-100 μg/kg, 100 μg/kg-500 μg/kg, 500 μg/kg-1 mg/kg, 1 mg/kg-50 mg/kg, 50 mg/kg-100 mg/kg, 100 mg/kg-500 mg/kg (body weight of recipient), although a lower or higher dosage may be administered. Dosages as low as about 1.0 mg/kg may be expected to show some efficacy. Preferably, about 5 mg/kg is an acceptable dosage, although dosage levels up to about 50 mg/kg are also preferred especially for therapeutic use. Alternatively, administration of a specific amount of the binding molecule may be given which is not based upon the weight of the patient such as an amount in the range of 1 μg-100 μg, 1 mg-100 mg, or 1 gm-100 gm. For example, site specific administration may be to body compartment or cavity such as intrarticular, intrabronchial, intraabdominal, intracapsular, intracartilaginous, intracavitary, intracelial, intracelebellar, intracerebroventricular, intracolic, intracervical, intragastric, intrahepatic, intramyocardial, intraosteal, intrapelvic, intrapericardiac, intraperitoneal, intrapleural, intraprostatic, intrapulmonary, intrarectal, intrarenal, intraretinal, intraspinal, intrasynovial, intrathoracic, intrauterine, intravesical, intralesional, vaginal, rectal, buccal, sublingual, intranasal, ophthalmic, or transdermal means.

The binding molecule composition can be prepared for use for parenteral (subcutaneous, intramuscular or intravenous) or any other administration particularly in the form of liquid solutions or suspensions; for use in vaginal or rectal administration particularly in semisolid forms such as, but not limited to, creams and suppositories; for buccal, or sublingual administration such as, but not limited to, in the form of tablets or capsules; or intranasally such as, but not limited to, the form of powders, nasal drops or aerosols or certain agents; or ophthalmically such as, but not limited to, eye drops; or for the treatment of dental disease; or transdermally such as not limited to a gel, ointment, lotion, suspension or patch delivery system with chemical enhancers such as dimethyl sulfoxide to either modify the skin structure or to increase the drug concentration in the transdermal patch, or with oxidizing agents that enable the application of formulations containing proteins and peptides onto the skin (WO 98/53847), or applications of electric fields to create transient transport pathways such as electroporation, or to increase the mobility of charged drugs through the skin such as iontophoresis, or application of ultrasound such as sonophoresis (U.S. Pat. Nos. 4,309,989 and 4,767,402).

The binding molecules of the present invention can be formulated according to known methods to prepare pharmaceutically useful compositions, whereby these materials, or their functional derivatives, are combined in admixture with a pharmaceutically acceptable carrier vehicle. Suitable vehicles and their formulation, inclusive of other human proteins, e.g., human serum albumin, are described, for example, in Remington's Pharmaceutical Sciences (16th ed., Osol, A. ed., Mack Easton Pa. (1980)). In order to form a pharmaceutically acceptable composition suitable for effective administration, such compositions will contain an effective amount of the above-described compounds together with a suitable amount of carrier vehicle. Additional pharmaceutical methods may be employed to control the duration of action. Controlled release preparations may be achieved through the use of polymers to complex or absorb the compounds. Another possible method to control the duration of action by controlled release preparations is to incorporate the compounds of the present invention into particles of a polymeric material such as polyesters, polyamino acids, hydrogels, poly(lacticacid) or ethylene vinylacetate copolymers. Alternatively, instead of incorporating these agents into polymeric particles, it is possible to entrap these materials in microcapsules prepared, for example, interfacial polymerization, for example, hydroxymethylcellulose or gelatin-microcapsules and poly(methylmethacylate)-microcapsules, respectively, or in colloidal drug delivery systems, for example, liposomes, albumin microspheres, microemulsions, nanoparticles, and nanocapsules or in macroemulsions.

The treatment may be given in a single dose schedule, or preferably a multiple dose schedule in which a primary course of treatment may be with 1-10 separate doses, followed by other doses given at subsequent time intervals required to maintain and or reinforce the response, for example, at 1-4 months for a second dose, and if needed, a subsequent dose(s) after several months. Examples of suitable treatment schedules include: (i) 0, 1 month and 6 months, (ii) 0, 7 days and 1 month, (iii) 0 and 1 month, (iv) 0 and 6 months, or other schedules sufficient to elicit the desired responses expected to reduce disease symptoms, or reduce severity of disease.

### Methods of Diagnosis

In some embodiments, the presence of a protein in a subject's cell, tissue, or bodily fluid is used in the methods of the invention as marker for the diagnosis of a disease or disorder (e.g., SARS-CoV-2 infection or COVID-19), assessing the severity of a disease or disorder, and for monitoring the effect or effectiveness of a treatment of a disease or disorder.

In one embodiment, the invention is a method of diagnosing a disease or disorder by assessing the level of at least one SARS-CoV-2 Nsp in a subject. Non-limiting examples of this embodiment would be contacting an affinity substrate comprising a SARS-CoV-2 Nsp binding molecule of the invention with a biological sample from a subject, and determining the binding of the SARS-CoV-2 Nsp to the SARS-CoV-2 Nsp binding molecule. In such examples, the SARS-CoV-2 Nsp binding molecule of the invention may be fused or conjugated to a detection moiety. These moieties may include (but are not limited to) a radioisotope, a magnetic spin-label, a fluorophore, a fluorescent protein, or a bioluminescent protein.

In one embodiment, the invention is a method of diagnosing a disease or disorder of a subject by assessing the level of at least one SARS-CoV-2 Nsp, in a biological sample of the subject. In one embodiment, the biological sample of the subject is a cell, tissue, or bodily fluid. Non-limiting examples of bodily fluids in which the level of at least one SARS-CoV-2 Nsp can be assessed include, but are not limited to, nasal mucus, saliva, blood, serum, plasma and urine. In various embodiments, the level of at least one SARS-CoV-2 Nsp, in the biological sample of the subject is compared with the SARS-CoV-2 Nsp level in a comparator. Non-limiting examples of comparators include, but are not limited to, a negative control, a positive control, an expected normal background value of the subject, a historical normal background value of the subject, an expected normal background value of a population that the subject is a member of, or a historical normal background value of a population that the subject is a member of. In some embodiments, the method of diagnosing includes a further step of treating the patient for the diagnosed disease or disorder (e.g., SARS-CoV-2 infection).

In another embodiment, the invention is a method of assessing the severity of a disease or disorder of a subject by assessing the level of at least one SARS-CoV-2 Nsp in a biological sample of the subject. In one embodiment, the biological sample of the subject is a cell, tissue, or bodily fluid. Non-limiting examples of bodily fluids in which the level of at least one SARS-CoV-2 Nsp can be assessed include, but are not limited to, nasal mucus, saliva, blood, serum, plasma and urine.

In another embodiment, the invention is a method of monitoring the effect of a treatment of a disease or disorder of a subject by assessing the level of at least one SARS-CoV-2 Nsp in a biological sample of the subject. In one embodiment, the biological sample of the subject is a cell, tissue, or bodily fluid. Non-limiting examples of bodily fluids in which the level of at least one SARS-CoV-2 Nsp can be assessed include, but are not limited to, nasal mucus, saliva, blood, serum, plasma and urine.

In various embodiments, the subject is a human subject, and may be of any race, sex and age. Representative subjects include those who are suspected of having experienced a disease or disorder, those who have been diagnosed as having experienced a disease or disorder, those who have been diagnosed as having a disease or disorder, and those who are at risk of developing a disease or disorder.

Information obtained from the methods of the invention described herein can be used alone, or in combination with other information (e.g., disease status, disease history, vital signs, blood chemistry, etc.) from the subject or from the biological sample obtained from the subject.

In the diagnostic methods of the invention, a biological sample obtained from a subject is assessed for the level of at least one SARS-CoV-2 Nsp contained therein. In one embodiment, the biological sample is a sample containing at least a fragment of a SARS-CoV-2 Nsp useful in the methods described herein.

In other various embodiments of the methods of the invention, the level of at least one SARS-CoV-2 Nsp is determined to be increased when the level of at least one SARS-CoV-2 Nsp is increased by at least 10%, by at least 20%, by at least 30%, by at least 40%, by at least 50%, by at least 60%, by at least 70%, by at least 80%, by at least 90%, by at least 100%, by at least 200%, by at least 300%, by at least 400%, by at least 500%, by at least 600%, by at least 700%, by at least 800%, by at least 900%, by at least 1000%, when compared to with a comparator control. In various embodiments, an increased level of at least one SARS-CoV-2 Nsp is indicative of an active disease or disorder.

Methods of measuring SARS-CoV-2 Nsp levels in a biological sample obtained from a patient include, but are not limited to, an immunochromatography assay, an immunodot assay, a Luminex assay, an ELISA assay, an ELISPOT assay, a protein microarray assay, a Western blot assay, a mass spectrophotometry assay, a radioimmunoassay (RIA), a radioimmunodiffusion assay, a liquid chromatography-tandem mass spectrometry assay, an ouchterlony immunodiffusion assay, reverse phase protein microarray, a rocket immunoelectrophoresis assay, an immunohistostaining assay, an immunofluorescence assay, an immunoprecipitation assay, a complement fixation assay, FACS, flow cytometry, an enzyme-substrate binding assay, an enzymatic assay, an enzymatic assay employing a detectable molecule, such as a chromophore, fluorophore, or radioactive substrate, a isotopic label, a substrate binding assay employing such a substrate, a substrate displacement assay employing such a substrate, and a protein chip assay (see also, 2007, Van Emon, Immunoassay and Other Bioanalytical Techniques, CRC Press; 2005, Wild, Immunoassay Handbook, Gulf Professional Publishing; 1996, Diamandis and Christopoulos, Immunoassay, Academic Press; 2005, Joos, Microarrays in Clinical Diagnosis, Humana Press; 2005, Hamdan and Righetti, Proteomics Today, John Wiley and Sons; 2007). In some embodiments, the level of SARS-CoV-2 Nsp9 in the biological sample is measure with an assay that uses at least one of the SARS-CoV-2 Nsp binding molecules of the invention that are described elsewhere herein.

### Kits

The present invention also provides kits which are useful for carrying out the present invention. In some embodiments, the kit comprises one or more SARS-CoV-2 Nsp binding molecule of the invention and an instructional material which describes, for instance, administering the SARS-CoV-2 Nsp binding molecule to an individual as a therapeutic treatment or use of the SARS-CoV-2 Nsp binding molecule in an assay as described elsewhere herein.

The present kits comprise a first container containing or packaged in association with the above-described nanobodies. The kit may also comprise another container containing or packaged in association solutions necessary or convenient for carrying out the invention. The containers can be made of glass, plastic or foil and can be a vial, bottle, pouch, tube, bag, etc. The kit may also contain written information, such as procedures for carrying out the present invention or analytical information, such as the amount of reagent contained in the first container means. The container may be in another container apparatus, e.g. a box or a bag, along with the written information.

Yet another aspect of the present invention is a kit for detecting at least one SARS-CoV-2 Nsp in a biological sample. In some embodiments, the kit includes a container, substrate or cartridge holding one or more SARS-CoV-2 Nsp binding molecule which binds an epitope of its target protein and instructions for using the SARS-CoV-2 Nsp binding molecule for the purpose of binding to SARS-CoV-2 Nsp to form a complex, and detecting the formation of the complex, such that the presence or absence of the complex correlates with presence or absence of the SARS-CoV-2 Nsp in the sample. Examples of containers include, but are not limited to, multi-well plates and single use devices containing a substrate comprising at least one SARS-CoV-2 Nsp binding molecule of the invention.

In some embodiments, the kit further comprises a carrier suitable for dissolving or suspending the SARS-CoV-2 Nsp or a sample comprising the SARS-CoV-2 Nsp, or combinations thereof, of the invention, for instance, a pharmaceutically acceptable carrier for dissolving or suspending a sample comprising SARS-CoV-2 Nsp9 prior to contacting the sample with a SARS-CoV-2 Nsp9 binding molecule of the invention. Optionally, the kit comprises an applicator for collecting and/or administering a sample comprising at least one SARS-CoV-2 Nsp to the substrate comprising the SARS-CoV-2 Nsp binding molecule of the invention.

### EXPERIMENTAL EXAMPLES

The invention is now described with reference to the following Examples. These Examples are provided for the purpose of illustration only and the invention should in no way be construed as being limited to these Examples, but rather should be construed to encompass any and all variations which become evident as a result of the teaching provided herein.

Without further description, it is believed that one of ordinary skill in the art can, using the preceding description and the following illustrative examples, make and utilize the compounds of the present invention and practice the claimed methods. The following working examples therefore, specifically point out the preferred embodiments of the present invention, and are not to be construed as limiting in any way the remainder of the disclosure.

### Example 1: NMR-Based Analysis of Nanobodies to SARS-CoV-2 Nsp9 Reveals a Possible Antiviral Strategy Against COVID19

Nanobodies are the variable domains of heavy chain anti-bodies (HCAbs), a component of the antibody repertoire of camelids, binding their antigens also when used as single domains devoid of the constant HCAbs frame (Hamers-Casterman et al., 1993, Nature, 363:446-448). Several nanobodies have been generated against the surface-ex-posed portion of Spike with the aim of blocking viral entry in the host cell (Hanke et al., 2020, Nature Communications, 11:4420; Koenig et al., 2021, Science, 371:eabe6230). Here, experiments are presented to identify other potential targets for development of nanobodies that could have potential use in diagnostics and, possibly, treatment. The multi-subunit Replication Transcription Complex (RTC), whose subunits are encoded by two large open reading frames (ORFs) (Yan et al., 2020, Nature Communications, 11:5874) was targeted. Recently, structural snapshots of the SARS-CoV-2 RTC have been reported at atomic resolution. The complex is assembled by Nsp7-(Nsp8)2-Nsp12-(Nsp13)2-RNA and a single RNA-binding protein, Nsp9, which is necessary for RTC function (Yan et al., 2020, Nature Communications, 11:5874). Although Nsp9 has a strong tendency to oligomerize (Ponnusamy et al., 2008, Journal of molecular biology, 383:1081-10965; Zhang et al., 2020, Molecular Biomedicine, 1:5; Miknis et al., 2009, Journal of virology, 83:3007-3018), within the RTC it is in a monomeric state (Yan et al., 2021, Cell, 184:184-193.e110). As a monomer, Nsp9 interacts with the Nsp12 (RdRp) NiRAN catalytic domain, which has nucleoside monophosphate (NMP) transferase activity, leading to the covalent attachment of a nucleoside monophosphate to the evolutionarily conserved Nsp9 amino terminus, a critical step in the initiation of viral replication (Slanina et al., 2021, Proceedings of the National Academy of Sciences, 118:e2022310118).

The experiments described herein demonstrate that 2NSP23 and 2NSP90 specifically interact with wild type Nsp9 and favor tetramerization of the protein. As this is not compatible with its monomeric configuration within the RTC complex (Yan et al., 2021, Cell, 184:184-193.e110), those nanobodies may contribute to the inhibition of viral replication by forcing the protein in a state that is not suitable for its RTC recruitment. By perturbing the monomer-dimer-tetramer transition toward the induction of a stable tetramer, we therefore speculate that 2NSP23 and 2NSP90 may serve as a possible Nsp9 inhibitor, negatively impacting on SARS-CoV-2 replication.

The materials and methods used in the experiments are now described

triSer-Nsp9 Protein Preparation.

Recombinant wild-type (wt) Nsp9 and triSer-Nsp9 proteins carrying the three amino acid substitutions C14S, C23S, C73S were provided by ASLA Biotech (Lot No. 2007ZJ03NSP01). Unlabeled and uniformly ¹⁵N,¹³C-labelled SARS-CoV-2 C14S,C23S,C73S NSP9 (triSer-NSP9) were expressed with an additional methionine at the N-terminus (M0). A construct was used for expression where the his-tag is located after the stop codon and therefore is not expressed. The scheme for this genetic construct was: pET22b(NdeI)-NSP9(113 aa+M0)-pET22b(XhoI) where CATATG is for NdeI; CTCGAG for XhoI; TAA is a stop codon, corresponding to DNA sequence:

(SEQ ID NO: 97)
CATATGAACAACGAACTGAGTCCGGTGGCACTGCGTCAGATGAGTA
GTGCCGCCGGCACCACCCAGACCGCCAGTACAGATGATAATGCCC
TGGCCTATTATAATACCACCAAAGGTGGTCGCTTTGTTCTGGCAC
TGCTGAGCGATCTGCAGGATCTGAAATGGGCACGTTTTCCGAAAA
GCGATGGTACCGGCACCATCTATACCGAACTGGAACCGCCGAGTC
GTTTTGTGACCGATACCCCGAAAGGTCCGAAAGTGAAATATCTGT
ATTTTATTAAGGGTCTGAACAACCTGAATCGTGGTATGGTGCTGG
GTAGTCTGGCAGCAACCGTTCGCCTGCAGTAACTCGAGCACCACC
ACCACCACCACTGA.

After IPTG induction of the Escherichia coli BL21 (DE3) transformed strain, the cells were grown at 22° C. for 18 hours in M9 medium containing 13C-labeled glucose and 15N-labeled ammonium chloride. The cell pellet was collected by centrifugation and resuspended in lysis buffer (1/40 volume: 1×PBS, protease inhibitor cocktail), sonicated and then centrifuged at 32000×g for 30 min. The cell pellet was dissolved in 6M GuCl, 50 mM NaPi, pH 7.3 solution and incubated for 40 min. The cell lysate was again centrifuged at 32000×g. The recombinant protein was further purified by cation exchange chromatography using a HiTrap SP HP column (GE Healthcare, 20 ml). The protein rich fractions were pooled, concentrated and applied to a Superdex 200 size-exclusion column (GE Healthcare, 120 ml). The protein was refolded by drop dilution into refolding buffer (100 mM Tris, 5 mM EDTA, 0.75M arginine, pH 8) on ice. The protein was further dialyzed twice against PBS (1:40 volume), and then again concentrated and dialyzed by Amicon Ultra 15 (Merck Millipore) against 20 mM ammonium acetate. The final protein preparation was lyophilized.

The uniformly ¹⁵N, ¹³C-labelled wild-type SARS-CoV-2 NSP9 was obtained according to the protocol kindly provided by the NMR COVID-19 Consortium (Dudás et al., 2021, Biomolecular NMR Assignments, doi: 10.1007/s12104-021-10011-0), leading to a final product with an additional Gly AlaMetGly tetrapeptide at the N-terminus.

For immunization, about 3 mg of lyophilized triSer-Nsp9 protein were dissolved in 20 mM ammonium acetate (pH 6.7). As the lyophilized protein was not fully dissolved in this buffer, the supernatant obtained by centrifugation, was recovered and stored for further use in immunization, panning and ELISA screening. The protein pellet (insoluble protein fraction) was dissolved in a small amount of dimethyl sulfoxide (DMSO), and the protein in DMSO was immediately diluted in PBS. Both the supernatant obtained by dissolving the protein in ammonium acetate, followed by centrifugation, and the protein dissolved in DMSO & diluted in PBS were used separately in the immunization, panning and ELISA screening experiments described below. In these experiments, we refer to the protein in ammonium acetate as NSP, and the protein dissolved in DMSO and diluted in PBS as INS.

### Immunization.

A llama was subcutaneously injected on days 0, 7, 14, 21, 28 and 35, each time with about 100 μg of recombinant triSer-NSP9 dissolved in ammonium acetate (here referred to as NSP) & 100 μg of recombinant triSer-NSP9 protein dissolved in DMSO/PBS (here referred to as INS). Each injection was performed at 2 sites at the end of the neck (cranially or caudally to the scapula bone) and at 2 sites at the two back limbs. The protein dissolved in ammonium acetate (NSP) was injected on the left side of the animal and the protein dissolved in DMSO/PBS (INS) was injected on the right side of the animal. The adjuvant used was Gerbu adjuvant P. On day 40 (5 days after last immunization), about 100 ml anticoagulated blood was collected from the llama for peripheral blood lymphocytes (PBLs) preparation & library generation.

### Construction of a VHH (Nanobody) Library.

A VHH library was constructed from PBLs to screen for the presence of antigen-specific nanobodies. To this end, total RNA was prepared from PBLs and about 50 μg of total RNA was used as template for first strand cDNA synthesis with oligodT primers. Using this cDNA, the VHH encoding sequences were amplified by PCR, digested with PstI and NotI, and cloned into the PstI and NotI sites of the phagemid vector pMECS. In pMECS vector, the nanobody sequence is followed by a linker, HA tag and His6 tag (Nanobody-AAAYPYDVPDYGSHHHHHH (SEQ ID NO: 98). Electro-competent E. coli TG1 cells were transformed with the recombinant pMECS vector resulting in a VHH (nanobody) library of about 2×10⁹ independent transformants. This library is named “Core 145/146 library”. About 87% of the transformants from this library harbored the vector with the right insert size (size of VHH-encoding sequences), as demonstrated by PCR analysis of 95 randomly picked colonies.

### Nanobodies Isolation.

The Core 145/146 library was panned separately on NSP & INS protein samples immobilized on solid-phase (100 μg/ml in 100 mM NaHCO₃ pH 8.2). Three rounds of panning were performed on each protein sample. The enrichment for antigen-specific phages was assessed after each round of panning by comparing the number of phagemid particles eluted from antigen-coated wells with the number of phagemid particles eluted from negative control (uncoated blocked) wells.

For panning on protein sample dissolved in ammonium acetate (NSP), the phage population was enriched for antigen-specific phages about 20-fold, 100-fold and 700-fold after the 1st, 2nd and 3rd round, respectively. In total, 190 colonies from 2nd and 3rd rounds (95 colonies from each round) were randomly selected and analyzed by ELISA for the presence of antigen-specific Nanobodies in their periplasmic extracts (ELISA using crude periplasmic extracts including soluble Nanobodies). The ELISA tests were performed on both the protein dissolved in ammonium acetate (NSP) and the protein dissolved in DMSO/PBS (INS). Uncoated blocked wells served as negative control (blank) for ELISA. Out of these 190 colonies, 165 colonies scored positive for NSP and/or INS. Based on sequence data of the 165 positive colonies, 71 different Nanobodies were identified, belonging to 18 different CDR3 groups (B-cell lineages). Out of these 71 Nanobodies, 65 Nanobodies are positive for both NSP and INS, while 6 Nanobodies bind only to NSP (See Excel file). The 71 different nanobodies specific for Nsp9 which resulted from panning on protein dissolved in ammonium acetate bear the code “NSP” in their names.

For panning on protein sample dissolved in DMSO/PBS (INS), the phage population was enriched for antigen-specific phages about 30-fold and 80-fold after the 2nd and 3rd round, respectively. No clear enrichment was observed after the 1st panning round. Here also, 190 colonies from 2nd and 3rd rounds (95 colonies from each round) were randomly selected and analyzed by ELISA for the presence of antigen-specific Nanobodies in their periplasmic extracts (ELISA using crude periplasmic extracts including soluble Nanobodies). The ELISA tests were performed on both the protein dissolved in ammonium acetate (NSP) and the protein dissolved in DMSO/PBS (INS). Uncoated blocked wells served as negative control (blank) for ELISA. Out of these 190 colonies, 156 colonies scored positive for INS and/or NSP. Based on sequence data of the 156 positive colonies, 65 different nanobodies were identified, belonging to 22 different CDR3 groups (B-cell lineages). Out of these 65 nanobodies, 59 nanobodies are positive for both INS and NSP, while 2 nanobodies are only positive for INS and 4 nanobodies bind only to NSP (See Excel file). The 65 different nanobodies specific for SARS-CoV-2 Nsp9 which resulted from panning on protein dissolved in DMSO/PBS bear the code “INS” in their names.

In summary, the above-described experiments resulted in 136 different nanobodies, belonging to 40 different CDR3 groups (B-cell lineages). Out of these 136 nanobodies, 124 specifically recognize both the protein dissolved in ammonium acetate and the protein dissolved in DMSO/PBS, while 12 nanobodies recognize either of these protein samples. The most likely explanation for differential binding of the later 12 nanobodies to 2 different protein samples is the experimental variabilities since the ELISA data here are all from single experiments. This is strongly supported by the fact that within the same CDR3 group (the same target epitope), there are both nanobodies binding to two different protein samples and also nanobodies which bind only to one protein sample. nanobodies belonging to the same CDR3 group (same B-cell lineage) are very similar and their amino acid sequences suggest that they are from clonally-related B-cells resulting from somatic hypermutation or from the same B-cell but diversified due to RT and/or PCR error during library construction. Nanobodies belonging to the same CDR3 group recognize the same epitope but their other characteristics (e.g. affinity, potency, stability, expression yield, etc.) can be different.

### Expression and Purification of Nanobodies.

Each nanobody was expressed in about 2 L of TB medium (6×330 ml). Small scale overnight cultures were started in 20 mL of LB medium supplemented with ampicillin (100 μg/ml) and glucose (1%). The overnight cultures were used to inoculate the shaker flasks, each containing 330 mL of TB medium supplemented with ampicillin (100 μg/ml) and glucose (0.1%), and grown at 220 rpm at 30° C. After the cultures reached an OD600 nm of about 0.8, expression was induced by addition of IPTG to final concentration of 1 mM. The induced cultures were then incubated at 20° C. at 220 rpm. The following day, the cultures were spun down. The supernatants were discarded and the cell pellets were subject to osmotic shock by resuspending each pellet from 330 ml culture in 4 mL TES buffer & incubating for 2 h at 4° C. with gentle shaking, followed by addition of 8 mL of water and further 4 h incubation at 4° C. The periplasmic extracts (PEs) were collected by centrifugation and stored at 4° C. The cell pellets were used for a second osmotic shock cycle. When all extracts were collected, 500 μL of His-select matrix was added to each individual periplasmic extract (PE) and incubated for 1 h at 4° C. Each PE was then applied to an empty PD-10 column. The flow through was applied to the column for a second time. Subsequently, the matrix was washed with a solution of 20 mM imidazole in PBS. Finally, the bound Nanobodies were eluted using a solution of 500 mM imidazole in PBS, in 5 fractions of 1 mL. These fractions were monitored by Nanodrop at 280 nm to quantify the amount of nanobody collected. The fractions containing substantial amounts of protein were pooled and loaded onto a Superdex 75 16/60 or a Superdex 75 10/300 GL Increase size exclusion chromatography (SEC) columns.

### Saliva Samples Collection and Storage.

Saliva samples from individuals with known SARS-CoV-2 status (positive and negative) were used for this study. Approximately 1 ml of saliva samples were collected in sterile 5 ml falcon tubes (Fisher scientific) without transport medium under NYUAD IRB-approved protocol HRPP-2020-48 (PI Idaghdour), stored directly at 4° C. until shipment to a BSL2 laboratory for processing following guidelines from the US Center for Disease Control and Prevention (CDC). The samples were stored at −80 C until processing.

### Saliva RNA Isolation and SARS-CoV-2 Detection by RT-qPCR.

For each sample, 300 μl of saliva were used to extract RNA in a BSL2 laboratory using an automated system, Chemagic 360, and Viral DNA/RNA 300 Kit H96, both from Perkin Elmer. Extracted RNA was eluted in 80 μL of elution buffer and used right away for SARS-Cov-2 detection or stored at −80° C. until use as per manufacturer's instruction protocol. The full protocol for SARS-CoV-2 detection using RT-qPCR and the Fluidigm BioMark HD system (Xie et al, 2020, Processes, 8, 1425). CDC recommended assays (primers and probes) for SARS-CoV-2 detection, and human RNase P (RP) control for RNA extraction and RT-qPCR reactions were used. The assays were synthesized by Integrated DNA Technology (IDT) and the sequences are as follow: 2019-nCoV_N2 (Forward: TTACAAACATTGGCCGCAAA (SEQ ID NO: 99); Reverse: GCGCGACATTCCGAAGAA (SEQ ID NO:100); Probe: ACAATTTGCCCCCAGCGCTTCAG (SEQ ID NO: 101); human RNase P (RP) (Forward: AGATTTGGACCTGCGAGCG (SEQ ID NO: 102); Reverse: GAGCGGCTGTCTCCACAAGT (SEQ ID NO: 103); Probe: TTCTGACCTGAAGGCTCTGCGCG (SEQ ID NO: 104). For each sample, five microliters of extracted RNA were reverse transcribed (RT) to cDNA using 1.25 μL RT Master Mix following the manufacturer protocol (Fluidigm). The converted cDNA was pre-amplified using a pool of the three assays (N1, N2, and RP) diluted into low TE buffer (Thermo Scientific) to a final concentration of 100.5 and 25.5 nM for the primers and probes, respectively, and then mixed with 2.5 μL of Preamplification Master Mix, and 0.635 μL PCR grade water (Fluidigm). After the pre-amplification step, the PCR reactions were further diluted 1:5 in Low TE buffer (Thermo Scientific) resulting in a final volume of 62.5 μL and ready to use for qPCR. The qPCR mix was prepared using 1.8 μL of diluted cDNA and 2 μL of 2× TaqMan Fast Advanced Master Mix (Thermo Scientific) and 0.2 μL 20×GE Sample Loading Reagent (Fluidigm). Next, 3 μL qPCR mix from each sample was loaded into the sample inlet in the 192.24 integrated fluid circuits (IFC, Fluidigm). For each assay, 3 μL of primer/probe mix (13.5×) was mixed with 1 μL of 4× Assay Loading Reagent (Fluidigm), and 3 μL of each assay mixed was loaded in the assay inlet in the 192.24 or Flex Six IFC chip (Fluidigm). The chip was then placed in an integrated fluidic circuit (IFC) controller RX machine to pre-load the samples and the assays, and then, loaded onto the BioMark HD instrument (Fluidigm) for RT-qPCR using 35 cycles. The raw amplification data were acquired using the Fluidigm data collection software and analyzed using the Fluidigm Real-Time PCR Analysis software 3.0.2. The expression of viral N2 protein was normalized to human RNase P expression and compared between samples. Each RT-qPCR reaction was repeated at least 4 times.

### Western Blot Analysis.

Purified wtNsp9 was diluted in 1×PBS buffer to prepare 1 mg/ml stock solution with protease inhibitors. The stock wtNsp9 solution was further diluted with 1×PBS containing 1 mg/ml bovine serum albumin (BSA) to the final concentration of 15 μg of total protein per loading with decreasing amount of wtNsp9 per sample. Following SDS PAGE, protein samples were visualized by PageBlue Protein Staining solution (Thermo Fisher) according to the manufacturer's instructions. For preparation of saliva samples, 20 μl of saliva was mixed with 2×RIPA buffer (50 mM Tris-HCl pH 7.5, 150 mM NaCl, 1 mM EDTA, 1% NP-40, 0.5% sodium deoxycholate, 0.1% SDS) in 1:1 ratio and total protein concentration measured by the Pierce BCA Assay Kit (Thermo Fisher Scientific). Subsequently 150 μl of the saliva sample was mixed with 50 μl of 4× Laemmli buffer with protease inhibitors and based on the protein concentration measurement by BCA assay, approximately 15 μg of total protein per sample was loaded to the 15% SDS-PAGE gel. The extracts were separated under reducing conditions and transferred to polyvinylidene difluoride (PVDF) membrane and blocked with 3% BSA in 1×TBST buffer (20 mM Tris, 150 mM NaCl, 0.1% Tween 20) for 1 hour. After blocking, membranes were incubated with nanobodies 2NSP23 and 2NSP90 (dilution 1000× in 1×TBST buffer) overnight at 4° C. and subsequently washed 4 times for 15 minutes in 1×TBST buffer. Immunoblots were then stained with HRP-conjugated secondary antibodies (dilution 2000× in 1×TBST buffer) recognizing either 6×His (ab237339, Abcam) or VHH epitopes (128-035-230, Jackson ImmunoResearch).

Detection was by chemiluminescence using the ECL Western Blot Substrate (Bio-Rad) and imaged by a ChemiDoc MP Imaging system (Bio-Rad).

### NMR Spectroscopy.

TCEP (tris(2-carboxyethyl)phosphine), sodium phosphate, hen egg white lysozyme (HEWL), D20 and DMSO-d6 were all from Sigma Aldrich (St. Louis, MO, USA). Perdeuterated glycine was from Cambridge Isotope Laboratories (Tewksbury, MA, USA). The uniformly ¹⁵N, ¹³C-labelled wild-type SARS-CoV-2 Nsp9 and triSer-Nsp9 were obtained as described in Experimental Protocols. The wild-type protein samples were typically prepared in H2O/D2O 95/5 with phosphate buffer 10-30 mM, 60-150 mM NaCl, 0.4-1.2 mM TCEP, 0.004-0.01% NaN₃, pH 7.0-7.1. For the triSer-Nsp9 samples, additional buffers and pH conditions were explored besides the phosphate (25 mM with 75 mM NaCl, pH 7.0). In particular, triSer-Nsp9 was first prepared and lyophilized from 20 mM ammonium acetate solution. Upon redissolving in H2O/D2O, pH was 6.1 (residual salt was observed) and the quality of the spectra was poor. Addition of saline phosphate buffer resulted in pronounced precipitation. This was also the case with the protein lyophilized from pure water that exhibited better solubility when dissolved without further additions (pH 4.7), or after dialysis against water (pH 3.7), or with 5% addition (in volume) of DMSO-d6, or in 100 mM perdeuterated glycine, pH 6.1. Under all these conditions, no major differences were detected with respect to phosphate, except for the solubility. In general, for the protein samples, concentrations ranged from 18 to 431 μM, as determined by UV absorption at 280 nm. No relevant difference was observed at different concentrations. For titrations with nanobodies (2NSP23 and 2NSP90) two mother solutions of 18 μM ¹⁵N,¹³C-labeled NSP9 without and with 36 μM unlabeled nanobody were progressively mixed. The buffer was 10 mM phosphate, 150 mM NaCl, 0.4 mM TCEP, 0.004% NaN₃ with pH 7.2. No precipitation was observed during the whole titrations. The NMR experiments of the isolated proteins were mostly acquired at 298 K. With triSer-NSP9 some experiments were also recorded at higher temperatures (305, 310 and 315 K) in the absence and presence of 5% DMSO-d6 to check for linewidth improvements. With the wild-type sequence the temperature was lowered to 278 and 276 K before starting the titrations which were in fact conducted at those two values with 2NSP23 and 2NSP90, respectively. The NMR data were collected at 14.0 T (1H at 600.19 MHz, 15N at 60.82 MHz) on a Bruker Avance III NMR system equipped with triple resonance cryoprobe. Two-dimensional 15N-1H HSQC experiments (Bodenhausen et al., 1980, Chem. Phys. Lett. 69:185-189) were carried out using sensitivity-improved Echo/Antiecho-TPPI pure phase detection in F1, gradient coherence selection and flip-back pulse for solvent suppression (Palmer et al., 1991, J. Magn. Reson. 93:151-170; Schleucher et al., 1994, J. Biomol. NMR, 4:301-306; Grzesiek et al., 1993, J. Am. Chem. Soc. 115:12593-12594). Data were collected over 40-50 ppm and 12-16 ppm windows in F1 and F2, respectively, using 128 points in t1 and 2,048 points in t2, with 32, 64 or 256 scans/t1 increment and 16 or 32 dummy scans to achieve steady state. Prior to 2D Fourier transform, squared sine-bell apodization, shifted at least by π/4 in either dimensions, and linear prediction to double the data size in t1 were applied, with zero filling to obtain 2K×1K matrices of reals. ¹³C was decoupled by band-selective adiabatic π pulses applied at the center of t1 evolution (Rosenfeld et al., 1997, J. Magn. Reson. 126:221-228). DOSY (Morris et al., 1992, J. Am. Chem. Soc. 114:3139-3141) spectra for determination of diffusion coefficients were acquired by 2D 1H DSTEBPP (Double STimulated Echo BiPolar Pulse) experiments with compensation for convective motions (Jerschow et al., 1998, J. Magn. Reson. 132:13-18). The z-axis gradient strength was varied linearly from 20 to 90% of its maximum value (˜60 G/cm) and matrices of 2,048 by 40 points were collected by accumulating 32-64 scans per gradient increment. Water suppression was achieved by appending to the DSTEBPP sequence a pair of WATERGATE (Piotto et al., 1992, J. Biomol. NMR, 2:661-665) elements in the excitation-sculpting mode (Hwang et al., 1995, J. Magn. Reson. A 112:275-279), as for 1D experiments. Following Fourier transform of the 1D traces, DOSY data fitting to extract the diffusion coefficients was carried out using Dynamics Center (Bruker). The related hydrodynamics calculations were done according to Hansen (Hansen, 2004, J. Chem. Phys. 121:9111-9115).

### MD Simulations.

The dimer of SARS-CoV-2 NSP9 RNA-Replicase was taken from the structure deposited in the Protein Data Bank (pdb id: 6w4b). The mutation of all cysteines into serines was performed using the program DeepView4.10 (Schwede et al., 2003, Nucl. Ac. Res. 31:3381-3385). The structures were soaked in a box of TIP3P water (Jorgensen et al., 1983, J. Chem. Phys. 79:926-935) and 0.150 M NaCl up to at least 14 Å from any solute atom using the program VMD (Humphrey et al., 1996, J. Mol. Graph. 14:33-38). All molecular simulations were performed using the program NAMD2 (Kale et al., 1999, J. Comp. Phys. 151:283-312). First each system was energy minimized by 5,000 steepest descent steps. The dynamics was started at 0 K and the temperature was increased to the target value in 10 ps and was equilibrated for 1 ns rescaling every 0.1 ps. During this phase and in all simulations pressure was kept constant at 1.0 atmosphere by the Langevin piston method with period 200 ps and decay time 100 ps, at the target temperature (Martyna et al., 1994, J. Chem. Phys. 101:4177-4189; Feller et al., 1995, J. Chem. Phys. 103:4613-4621). In all simulations, except for the heating phase described above, the temperature was controlled through Langevin dynamics with damping constant 2.5 ps−1. Interactions were gradually switched off at 12 Å starting at 10 Å. The time step was 1 fs for bonded interactions and 2 fs for nonbonded interactions. Hydrogen bond lengths were restrained by the algorithm Settle (Miyamoto et al., 1992, J. Comp. Chem. 13:952-962). Exactly the same protocol was applied to the wildtype and mutant protein. Simulations were performed for 200 ns.

The experimental results are now described

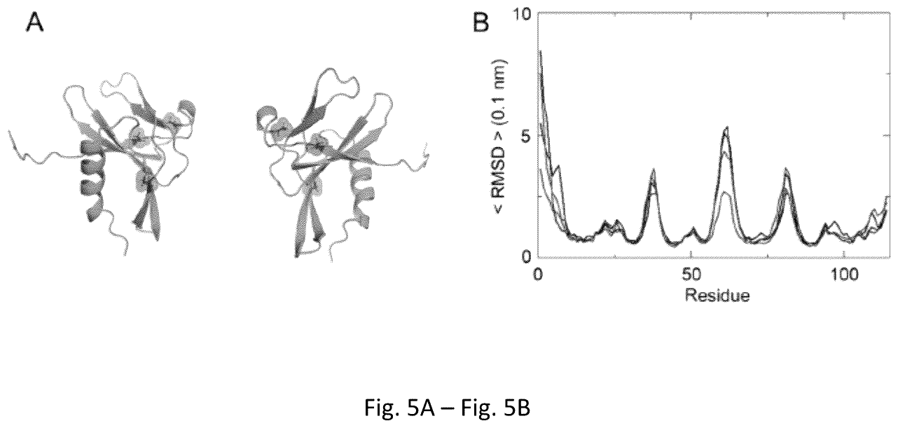

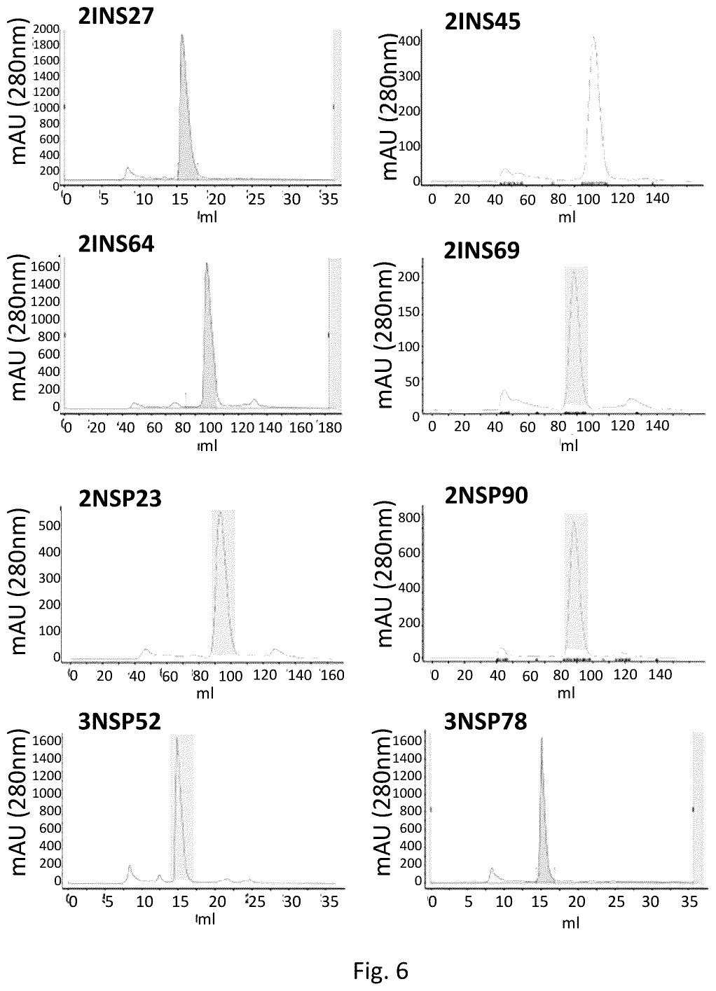

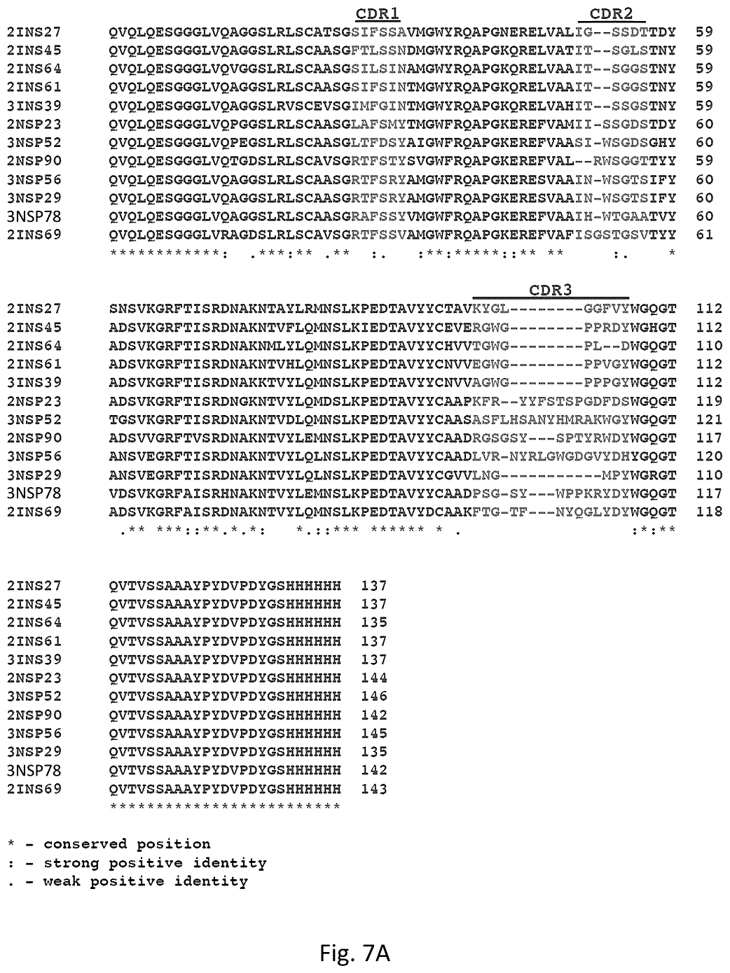

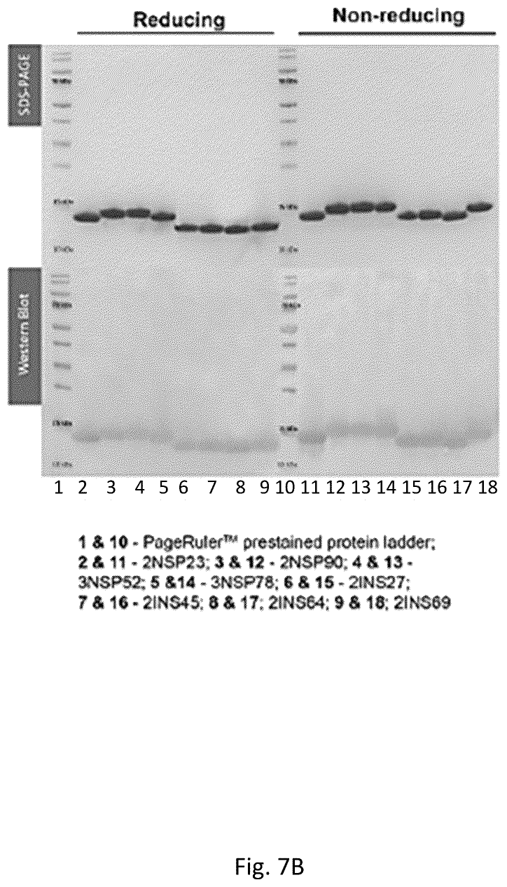

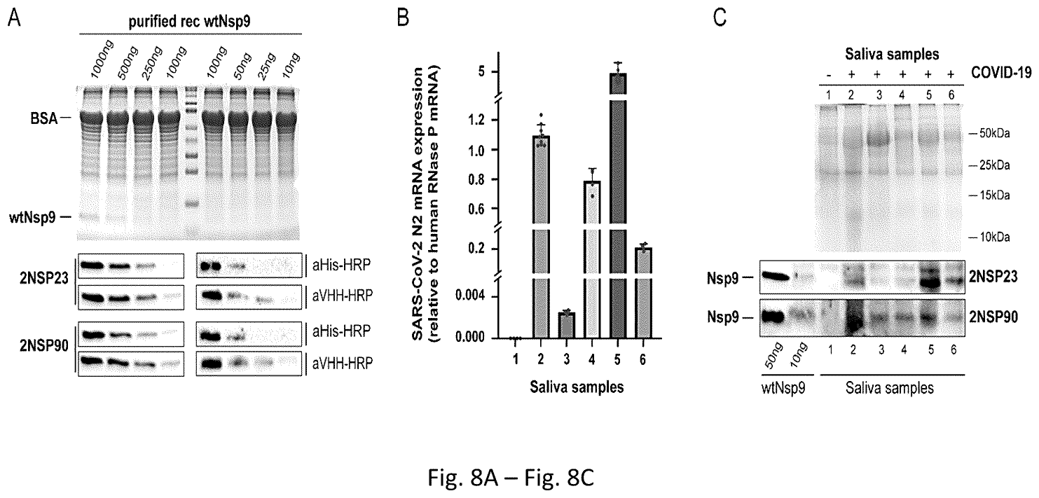

To select for nanobodies against Nsp9, a llama was immunized with a recombinant SARS-CoV-2 Nsp9 protein carrying three mutations, C14S, C23S, and C73S (triSer-Nsp9), to pre-vent oxidation of free Cys SH groups that could elicit heterogeneity in the immune response. Molecular dynamics simulations of wild-type and mutant Nsp9 show high similarity (FIG. 5). After the last immunization, anticoagulated blood was collected to prepare for peripheral blood lymphocytes (PBLs) preparation and library generation to screen for the presence of antigen-specific nanobodies. The details of the procedure are described in Supplementary Material. Overall, ELISA tests performed on immobilized triSer-Nsp9 identified 136 different nanobodies, belonging to 40 different CDR3 groups (B-cell lineages) (Table 1). Eight Nsp9-specific nanobody genes were selected from 8 different CDR3 groups. These genes were cloned, expressed in E. coli WK6 and purified by IMAC and size exclusion chromatography (FIG. 6). Sequences, annotations and analytical characterizations are given in FIG. 7 and Table 2. For further characterization, nanobodies 2NSP23 and 2NSP90 were selected and tested for binding to wild-type Nsp9 on immunoblots (FIG. 8A). After incubation with the membrane, nanobodies were detected with secondary antibodies recognizing either the llama VHH domain or His6 tag fused to both 2NSP23 and 2NSP90 nanobodies (FIG. 8A). Results from immunoblotting show that Nsp9 was specifically recognized by 2NSP23 and 2NSP90 at antigen concentrations as low as 25 ng per loading (1.25 ng/μl).

TABLE 1
The ELISA tests were performed on both Nsp9 dissolved in ammonium
acetate (NSP) and dissolved in DMSO/PBS (INS). Uncoated blocked
wells served as negative control (blank) for ELISA.

| | | | | ELISA | | |
|---|---|---|---|---|---|---|
| | CDR3 | ELISA | ELISA | blank | INS/ | NSP/ |
| Clone | Group | INS | NSP | (control) | control | control |
| 2INS6 | 1 | 1.6606 | 2.0241 | 0.0969 | 17.13725 | 20.88854 |
| 2INS13 | 1 | 3.6286 | 3.8515 | 0.4403 | 8.241199 | 8.747445 |
| 2INS14 | 1 | 3.1991 | 2.4905 | 0.1328 | 24.08961 | 18.75377 |
| 2INS15 | 1 | 3.5749 | 3.5556 | 0.1695 | 21.09086 | 20.97699 |
| 2INS21 | 1 | 3.8533 | 3.7983 | 0.1839 | 20.95324 | 20.65416 |
| 2INS26 | 1 | 4.8694 | 4.3108 | 0.0816 | 59.67402 | 52.82843 |
| 2INS27 | 1 | 2.9974 | 2.7664 | 0.0957 | 31.32079 | 28.907 |
| 2INS30 | 1 | 4.2339 | 2.9781 | 0.1347 | 31.43207 | 22.10913 |
| 2INS31 | 1 | 2.8974 | 1.5437 | 0.1004 | 28.85857 | 15.3755 |
| 2INS32 | 1 | 4.4284 | 3.9266 | 0.1341 | 33.02312 | 29.28113 |
| 2INS34 | 1 | 4.358 | 3.6778 | 0.1321 | 32.99016 | 27.84103 |
| 2INS35 | 1 | 2.2232 | 2.0145 | 0.1006 | 22.0994 | 20.02485 |
| 2INS37 | 1 | 4.2254 | 3.4193 | 0.1418 | 29.79831 | 24.11354 |
| 2INS42 | 1 | 3.9214 | 2.2874 | 0.1279 | 30.65989 | 17.88428 |
| 2INS66 | 1 | 2.3038 | 1.377 | 0.1035 | 22.25894 | 13.30435 |
| 2INS76 | 1 | 4.309 | 3.8965 | 0.3283 | 13.12519 | 11.86872 |
| 2INS91 | 1 | 4.6538 | 3.3368 | 0.4607 | 10.10158 | 7.242891 |
| 3INS1 | 1 | 1.7013 | 1.3189 | 0.0934 | 18.2152 | 14.12099 |
| 3INS6 | 1 | 1.6434 | 1.3272 | 0.0863 | 19.04287 | 15.37891 |
| 3INS7 | 1 | 1.0084 | 0.7275 | 0.0897 | 11.24192 | 8.110368 |
| 3INS8 | 1 | 0.9664 | 0.4178 | 0.0849 | 11.3828 | 4.921084 |
| 3INS11 | 1 | 2.2977 | 2.5381 | 0.093 | 24.70645 | 27.2914 |
| 3INS16 | 1 | 2.8449 | 2.1031 | 0.1085 | 26.22028 | 19.38341 |
| 3INS21 | 1 | 3.9952 | 2.8413 | 0.2329 | 17.15414 | 12.19966 |
| 3INS26 | 1 | 3.3184 | 3.0078 | 0.1104 | 30.05797 | 27.24457 |
| 3INS32 | 1 | 3.0735 | 2.1801 | 0.1536 | 20.00977 | 14.19336 |
| 3INS35 | 1 | 2.5429 | 1.9874 | 0.0964 | 26.37863 | 20.61618 |
| 3INS37 | 1 | 1.3301 | 1.2324 | 0.0867 | 15.34141 | 14.21453 |
| 3INS42 | 1 | 2.3328 | 1.7834 | 0.1437 | 16.23382 | 12.41058 |
| 3INS49 | 1 | 2.5399 | 2.9157 | 0.1203 | 21.11305 | 24.23691 |
| 3INS50 | 1 | 2.0109 | 1.4152 | 0.0906 | 22.19536 | 15.62031 |
| 3INS67 | 1 | 2.547 | 1.9937 | 0.1076 | 23.671 | 18.52881 |
| 3INS72 | 1 | 2.5229 | 1.8334 | 0.1063 | 23.73377 | 17.24741 |
| 3INS74 | 1 | 1.5646 | 0.7418 | 0.0868 | 18.02535 | 8.546083 |
| 3INS86 | 1 | 1.322 | 1.6495 | 0.289 | 4.574394 | 5.707612 |
| 3INS92 | 1 | 2.0624 | 1.8056 | 0.1 | 20.624 | 18.056 |
| 2NSP1 | 2 | 2.5 | 4.5831 | 0.0901 | 27.74695 | 50.86681 |
| 2NSP9 | 2 | 2.989 | 5.6897 | 0.096 | 31.13542 | 59.26771 |
| 2NSP33 | 2 | 0.5101 | 1.6461 | 0.0857 | 5.952159 | 19.2077 |
| 2NSP38 | 2 | 3.1907 | 4.952 | 0.0897 | 35.57079 | 55.20624 |
| 2NSP55 | 2 | 3.3116 | 5.1433 | 0.0934 | 35.4561 | 55.06745 |
| 2NSP62 | 2 | 1.9995 | 3.5031 | 0.0781 | 25.60179 | 44.85403 |
| 2NSP79 | 2 | 2.87 | 5.32 | 0.0973 | 29.4964 | 54.67626 |
| 2NSP80 | 2 | 4.2058 | 5.3673 | 0.0973 | 43.22508 | 55.16238 |
| 2NSP92 | 2 | 2.91 | 4.9596 | 0.0905 | 32.1547 | 54.80221 |
| 3NSP4 | 2 | 2.5069 | 3.7444 | 0.0987 | 25.39919 | 37.93718 |
| 3NSP12 | 2 | 2.1605 | 3.5488 | 0.0892 | 24.22085 | 39.78475 |
| 3NSP28 | 2 | 0.6466 | 1.9015 | 0.0828 | 7.809179 | 22.96498 |
| 3NSP33 | 2 | 2.2006 | 3.4793 | 0.0848 | 25.95047 | 41.02948 |
| 3NSP52 | 2 | 2.004 | 2.9065 | 0.0849 | 23.60424 | 34.23439 |
| 3NSP58 | 2 | 1.5084 | 2.3769 | 0.0839 | 17.97855 | 28.33015 |
| 3NSP64 | 2 | 1.6567 | 2.1431 | 0.0876 | 18.9121 | 24.46461 |
| 3NSP65 | 2 | 4.3901 | 4.72 | 0.1698 | 25.85453 | 27.79741 |
| 3NSP66 | 2 | 2.0413 | 2.721 | 0.0845 | 24.1574 | 32.20118 |
| 3NSP69 | 2 | 1.8129 | 2.6048 | 0.0866 | 20.93418 | 30.07852 |
| 3NSP70 | 2 | 0.2614 | 0.5126 | 0.087 | 3.004598 | 5.891954 |
| 3NSP71 | 2 | 1.4571 | 2.0619 | 0.0876 | 16.63356 | 23.53767 |
| 3NSP79 | 2 | 1.9037 | 2.877 | 0.0822 | 23.15937 | 35 |
| 2NSP10 | 3 | 1.4958 | 4.4567 | 0.0847 | 17.65998 | 52.61747 |
| 2NSP15 | 3 | 0.7356 | 3.5612 | 0.0887 | 8.293123 | 40.14882 |
| 2NSP20 | 3 | 3.9084 | 5.1347 | 0.0946 | 41.31501 | 54.27801 |
| 2NSP21 | 3 | 2.3299 | 5.1093 | 0.3555 | 6.553868 | 14.37215 |
| 2NSP24 | 3 | 1.6673 | 4.3661 | 0.1074 | 15.52421 | 40.6527 |
| 2NSP47 | 3 | 0.226 | 0.5769 | 0.0929 | 2.432723 | 6.209903 |
| 2NSP71 | 3 | 0.2482 | 0.8837 | 0.0853 | 2.90973 | 10.35991 |
| 2NSP72 | 3 | 0.1514 | 0.6148 | 0.0867 | 1.746251 | 7.091119 |
| 2NSP82 | 3 | 1.4239 | 4.4291 | 0.0905 | 15.7337 | 48.94033 |
| 2NSP85 | 3 | 0.2501 | 1.2002 | 0.0904 | 2.766593 | 13.27655 |
| 2NSP95 | 3 | 2.782 | 4.784 | 0.112 | 24.83929 | 42.71429 |
| 3NSP63 | 3 | 0.3468 | 0.7881 | 0.0808 | 4.292079 | 9.753713 |
| 3NSP73 | 3 | 1.7099 | 2.4623 | 0.0851 | 20.09283 | 28.9342 |
| 3NSP90 | 3 | 2.1166 | 2.9898 | 0.0862 | 24.55452 | 34.68445 |
| 2NSP16 | 4 | 4.5913 | 5.5139 | 0.2076 | 22.11609 | 26.56021 |
| 2NSP27 | 4 | 4.0351 | 5.2543 | 0.1565 | 25.78339 | 33.5738 |
| 2NSP28 | 4 | 3.5767 | 5.2162 | 0.1294 | 27.64065 | 40.31066 |
| 2NSP30 | 4 | 2.4645 | 4.7373 | 0.1052 | 23.42681 | 45.03137 |
| 3NSP22 | 4 | 1.9257 | 3.8951 | 0.1072 | 17.96362 | 36.33489 |
| 3NSP25 | 4 | 2.1565 | 3.5611 | 0.1081 | 19.94912 | 32.94265 |
| 3NSP36 | 4 | 1.9548 | 2.8852 | 0.0891 | 21.93939 | 32.38159 |
| 3NSP86 | 4 | 1.6768 | 2.4579 | 0.1002 | 16.73453 | 24.52994 |
| 2NSP22 | 5 | 0.9622 | 4.1327 | 0.0947 | 10.16051 | 43.63992 |
| 2NSP48 | 5 | 1.0245 | 3.7108 | 0.0926 | 11.06371 | 40.07343 |
| 2NSP90 | 5 | 1.9764 | 4.7805 | 0.0941 | 21.00319 | 50.80234 |
| 3NSP11 | 5 | 1.941 | 3.3478 | 0.0887 | 21.88275 | 37.74295 |
| 3NSP38 | 5 | 0.6673 | 1.7741 | 0.0869 | 7.678941 | 20.41542 |
| 3NSP51 | 5 | 0.7785 | 1.8501 | 0.0948 | 8.212025 | 19.51582 |
| 3NSP76 | 5 | 1.2509 | 0.6085 | 0.0836 | 14.96292 | 7.278708 |
| 2INS47 | 6 | 6 | 6 | 0.8376 | 7.163324 | 7.163324 |
| 3INS38 | 6 | 2.8893 | 4.6169 | 0.1378 | 20.96734 | 33.50435 |
| 3INS76 | 6 | 3.1719 | 4.9016 | 0.4658 | 6.809575 | 10.52297 |
| 2NSP44 | 7 | 0.8601 | 2.2453 | 0.1242 | 6.925121 | 18.0781 |
| 2NSP83 | 7 | 3.1988 | 4.981 | 0.4271 | 7.489581 | 11.66237 |
| 3NSP47 | 7 | 0.538 | 1.3658 | 0.1193 | 4.50964 | 11.44845 |
| 2NSP23 | 8 | 1.7184 | 4.2818 | 0.1025 | 16.76488 | 41.77366 |
| 2NSP25 | 8 | 3.1576 | 5.2541 | 0.0889 | 35.51856 | 59.10124 |
| 2NSP70 | 8 | 1.5966 | 3.2321 | 0.0938 | 17.02132 | 34.45736 |
| 2INS10 | 9 | 5.1786 | 4.5834 | 0.1207 | 42.90472 | 37.97349 |
| 2INS46 | 9 | 6 | 5.4499 | 0.1133 | 52.95675 | 48.1015 |
| 2INS61 | 10 | 1.8398 | 5.3567 | 0.6683 | 2.752955 | 8.015412 |
| 3INS39 | 10 | 2.493 | 4.0747 | 0.922 | 2.703905 | 4.419414 |
| 3NSP49 | 11 | 0.7918 | 1.4154 | 0.0846 | 9.359338 | 16.7305 |
| 3NSP59 | 11 | 1.8859 | 2.7246 | 0.0851 | 22.16099 | 32.01645 |
| 2INS24 | 12 | 4.8489 | 1.4099 | 0.0965 | 50.24767 | 14.61036 |
| 2INS55 | 12 | 4.8303 | 0.539 | 0.0848 | 56.96108 | 6.356132 |
| 2INS43 | 13 | 0.4183 | 2.0533 | 0.119 | 3.515126 | 17.25462 |
| 3INS17 | 13 | 0.7256 | 2.7437 | 0.0913 | 7.947426 | 30.05148 |
| 2INS70 | 14 | 2.0679 | 5.0097 | 0.1236 | 16.73058 | 40.53155 |
| 2INS95 | 14 | 4.784 | 5.1837 | 0.1089 | 43.93021 | 47.60055 |
| 2INS58 | 15 | 2.8069 | 5.0288 | 0.0989 | 28.38119 | 50.84732 |
| 3INS93 | 15 | 1.0681 | 4.1258 | 0.0882 | 12.10998 | 46.77778 |
| 3NSP48 | 16 | 1.5108 | 2.7245 | 0.1937 | 7.79969 | 14.06557 |
| 3NSP81 | 16 | 0.6041 | 0.6598 | 0.0831 | 7.269555 | 7.939832 |
| 3NSP29 | 17 | 0.2019 | 0.4232 | 0.1099 | 1.837125 | 3.850773 |
| 2INS12 | 18 | 1.0694 | 3.5463 | 0.2595 | 4.121002 | 13.6659 |
| 2INS57 | 19 | 1.6643 | 0.7977 | 0.2502 | 6.651878 | 3.188249 |
| 2INS64 | 20 | 2.6154 | 4.431 | 0.0981 | 26.66055 | 45.1682 |
| 2INS44 | 21 | 4.2208 | 5.3692 | 0.4552 | 9.272408 | 11.79525 |
| 2INS45 | 22 | 2.8379 | 4.7531 | 0.1056 | 26.87405 | 45.01042 |
| 2INS71 | 23 | 0.6416 | 2.9364 | 0.149 | 4.30604 | 19.70738 |
| 2NSP40 | 24 | 1.0389 | 2.3909 | 0.111 | 9.359459 | 21.53964 |
| 3NSP39 | 25 | 0.2981 | 0.7045 | 0.0962 | 3.098753 | 7.323285 |
| 2INS39 | 26 | 3.4962 | 5.479 | 0.8981 | 3.892885 | 6.100657 |
| 3INS61 | 27 | 0.8272 | 2.8749 | 0.3178 | 2.602895 | 9.046256 |
| 3NSP35 | 28 | 0.4166 | 1.0508 | 0.0907 | 4.593164 | 11.58545 |
| 3NSP78 | 29 | 2.3553 | 3.8839 | 0.0823 | 28.61847 | 47.19198 |
| 2INS69 | 30 | 1.3515 | 1.3234 | 0.0958 | 14.10752 | 13.8142 |
| 2NSP11 | 31 | 1.3861 | 5.9479 | 0.0902 | 15.36696 | 65.94124 |
| 2NSP84 | 32 | 0.4116 | 1.2763 | 0.112 | 3.675 | 11.39554 |
| 2INS84 | 33 | 1.0445 | 2.2204 | 0.5491 | 1.902204 | 4.043708 |
| 2INS19 | 34 | 0.8157 | 0.4392 | 0.0902 | 9.043237 | 4.86918 |
| 2INS33 | 35 | 0.4922 | 0.2021 | 0.0699 | 7.041488 | 2.891273 |
| 3INS19 | 36 | 0.6433 | 0.2598 | 0.0909 | 7.077008 | 2.858086 |
| 3NSP31 | 37 | 3.2462 | 3.936 | 0.0878 | 36.97267 | 44.82916 |
| 3NSP56 | 38 | 0.2368 | 0.4968 | 0.0908 | 2.60793 | 5.471366 |
| 2INS48 | 39 | 1.9693 | 2.925 | 0.3374 | 5.836692 | 8.669235 |
| 2NSP32 | 40 | 0.6162 | 1.5921 | 0.089 | 6.923596 | 17.88876 |

TABLE 2
Amino acid sequence analysis of the Nanobodies using the ProtParam tool allows
the prediction of several theorical proprieties. MW, Molecular Weight; pI,
isoelectric point; ε, extinction coefficient. Production yields of the
Nsp9-specific nanobodies in the expression vector pHEN6 are also included.

| | | CDR3 | | | ε (0.1%) | ε (0.1%) | |
|---|---|---|---|---|---|---|---|
| Peak loss | Nanobody | groups | Mw (Da) | pI | Cystines | Reduced | Yield |
| 23270 | 2NSP23 | 8 | 14435.99 | 6.64 | 1.493 | 1.484 | 8.29 |
| 23294 | 2NSP90 | 5 | 14404.76 | 8.00 | 2.467 | 2.458 | 7.45 |
| 23314 | 3NSP52 | 2 | 14614.12 | 8.03 | 2.126 | 2.117 | 2.21 |
| 23327 | 3NSP78 | 29 | 14258.86 | 8.96 | 2.388 | 2.379 | 1.74 |
| 23342 | 2INS27 | 1 | 13414.86 | 8.64 | 1.607 | 1.597 | 4.24 |
| 23354 | 2INS45 | 22 | 13636.08 | 8.03 | 1.766 | 1.756 | 4.57 |
| 23362 | 2INS64 | 20 | 13166.73 | 8.69 | 1.828 | 1.819 | 14.05 |
| 23364 | 2INS69 | 30 | 14289.82 | 8.63 | 1.508 | 1.500 | 3.14 |

Whether nanobodies 2NSP90 and 2NSP23 specifically bind Nsp9 in biological samples was next examined. Saliva from individuals infected with COVID-19 was collected in sterile containers, per a recent study demonstrating that saliva can be used for SARS-CoV-2 detection by RT-PCR (10). To confirm the presence of SARS-CoV-2 in the saliva samples, expression levels of mRNA encoding the viral N2 protein using real time qPCR were monitored. Significantly high N2 mRNA levels, normalized to expression of human RNase P mRNA, were observed in saliva from the five COVID-19 patients but not in a saliva sample from a healthy donor used as negative control (FIG. 8B). COVID-19 positive samples exhibited different N2 mRNA levels, compatible with different viral loads (FIG. 8B). To test if nanobodies 2NSP90 and 2NSP23 can detect Nsp9 in neat saliva from COVID-19 patients, proteins were extracted by diluting saliva samples in SDS loading buffer. Following heat denaturation, samples were electrophoresed under denaturing conditions and transferred on a membrane. For the immunoassays, membranes were separately incubated with nanobodies 2NSP90 and 2NSP23 followed by tagged secondary anti-VHH antibodies for visualization (FIG. 8C). A specific signal was detected across all COVID-19 patients' samples with both nanobodies. Neither 2NSP90 nor 2NSP23 exhibited a positive signal in the sample from the healthy donor, indicating a degree of specificity toward their antigen. The differences in the amounts of detected Nsp9 mirror differences in viral loads measured by real time qPCR. A comparison with purified Nsp9, loaded as control (50 ng and 10 ng), suggests that 2NSP90 and 2NSP23 can detect as little as 10 ng of Nsp9 protein in saliva.

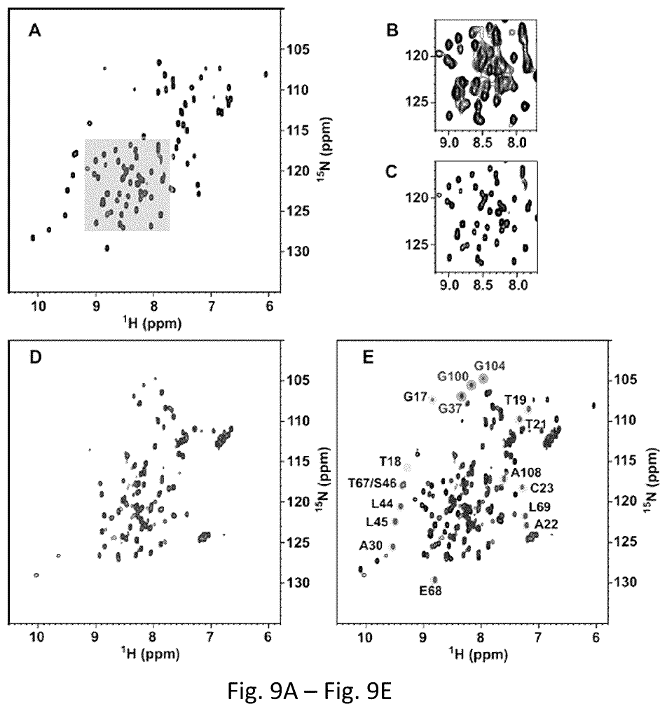

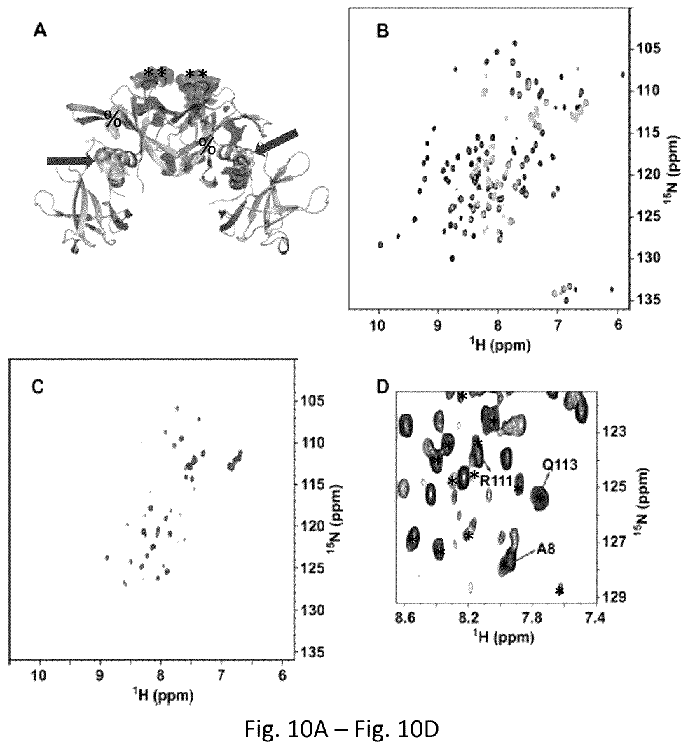

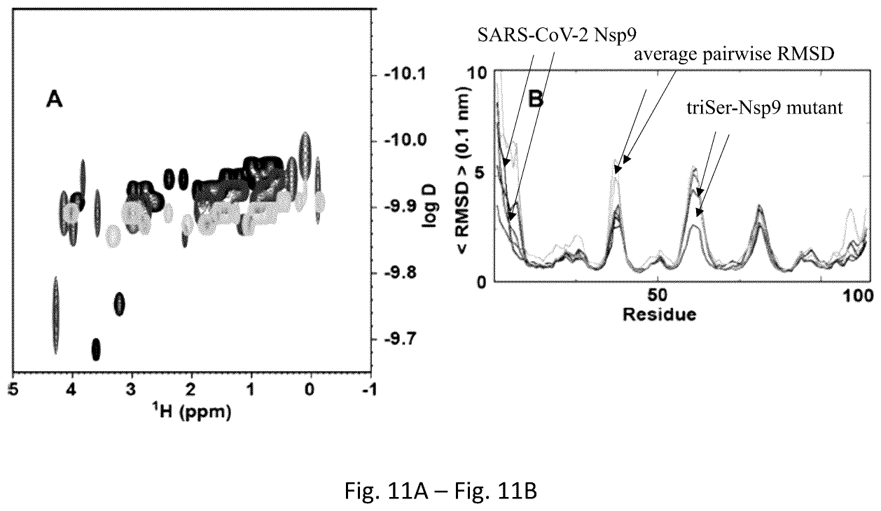

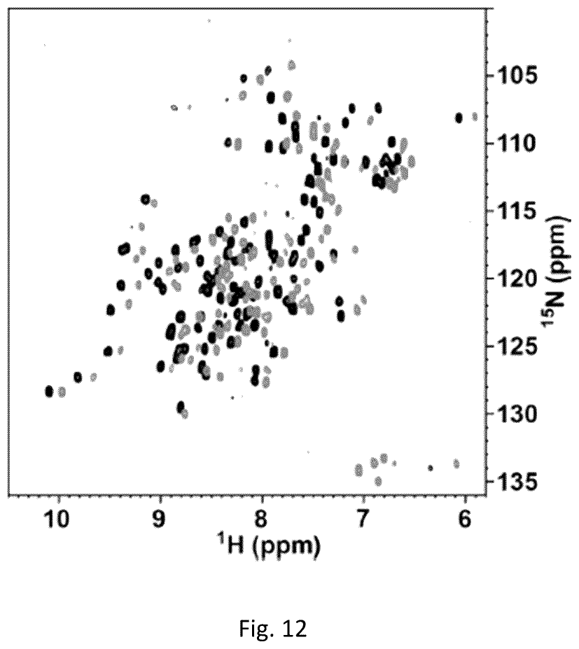

To begin characterizing the nature of the interaction of 2NSP90 and 2NSP23 with their antigen Nsp9, in-solution NMR spectroscopy was performed on wild-type Nsp9 and triSer-Nsp9. First, the ¹⁵N-¹H HSQC NMR spectrum of SARS-CoV-2 Nsp9 (FIG. 9A) was collected. The quality of the spectrum does not match the expectation for a protein of ˜13.4 kDa, the mass of our 13C,15-labeled Nsp9 construct. The cross-peaks are broadened (FIG. 9B and FIG. 9C) by the dimerization and possibly tetramerization that was anticipated from crystallographic evidence (Schleucher et al., 1994, J. Biomol. NMR, 4:301-306; Grzesiek et al, 1993, J. Am. Chem. Soc, 115:12593-12594; Rosenfeld et al., 1997, J. Magn. Reson, 126:221-228). This result is compatible with the recently reported NMR studies of SARS-CoV-2 Nsp9 (Buchko et al., 2021, Biomolecular NMR Assignments, 15:107-116; Dudás et al., 2021, Biomolecular NMR Assignments). ²H, ¹⁵N, ¹³C triply-labeled and selectively labeled samples were in fact necessary to improve coherence transfer in 3D experiments for backbone assignment (Buchko et al., 2021, Biomolecular NMR Assignments, 15:107-116). The 3D data confirmed poor coherence transfer for both wild-type Nsp9 and mutated triSer-Nsp9. The HSQC maps also show that the backbone amide connectivities from the residues of the dimerization interface (segments 1-7 and 96-106) are largely missing due to the intermediate exchange, on the chemical shift scale, of the dimerization process (Buchko et al., 2021, Biomolecular NMR Assignments, 15:107-116). An even more severe loss of cross-peaks affects the 15N-1H HSQC spectrum of the triSer-NSP9 mutant (FIG. 9D and FIG. 9E). Apart from the obvious lack of C14S, C23S and C73S cross-peaks, the signal loss of the mutant spectrum also concerns additional locations that significantly match the dimer-dimer interface of the tetramer (Zhang et al., 2020, Molecular Biomedicine, 1:5; Miknis et al., 2009, Journal of virology, 83:3007-3018). Therefore, the triSer-Nsp9 NMR spectrum reveals a further exchange implying the loss of the signals at the tetramerization interface because of an intermediate regime on the chemical shift scale, much like the dimerization ex-change observed also in the wild-type protein. In particular, cross-peak loss is seen for the stretches 67-69 and 17-22 of inter-dimer contact surface, whereas the stretches 30-32 and 44-46, whose signals also disappear in the triSer-Nsp9 spectrum, are located below that interface (FIG. 10A) and may report, therefore, the effect of a more distant conformational change related to tetramerization. Alternatively, this allosteric effect could reflect an additional response that maps to the monomer-monomer interface, as further inferred from the comparison of the HSQC spectra (FIG. 9E). The onset of the three cross-peak in the triSer-Nsp9 spectrum with the typical chemical shifts of glycine amides suggests that two of these signals could be tentatively assigned to G100 and G104, whereas the third is from G37, which is barely observed in wild-type Nsp9 but becomes well visible in the mutated triSer-Nsp9 probably because of dynamical changes induced by the proximity to the other dimer-dimer contact involving T35 and K36 (Zhang et al., 2020, Molecular Biomedicine, 1:5). The concurrent disappearance of A108 correlation in the triSer-Nsp9 spectrum, together with the involvement of the N- and C-terminal fragment along with the G100XXXG104 motif in the inter-monomer interface (Egloff et al., 2004, Proceedings of the National Academy of Sciences, 101:379213; Sutton et al., 2004, Structure, 12:341-353; Miknis et al., 2009, Journal of virology, 83:3007-3018; Zhang et al., 2020, Molecular Biomedicine, 1:5) suggest some rearrangement of this interface upon tetramerization (FIG. 10A). The higher extent of oligomerization in triSer-Nsp9 was further confirmed by NMR DOSY measurements of translational diffusion coefficient (D) (Morris et al., 1992, Journal of the American Chemical Society, 114:3139-3141; Jerschow et al., 1998, J. Magn. Reason, 132: 13-18) (FIG. 11). To study how 2NSP23 and 2NSP90 interact with Nsp9, we collected HSQC spectra of 15N-labeled wild-type Nsp9 upon titration with an unlabeled nanobody (Raimondi et al., 2017, Scientific Reports, 7:46711). The HSQC spectrum of Nsp9 shows the effect of the intermediate exchange between monomer and dimer that literally bleaches the am-ide cross-peaks of the residues in contact at the dimerization interface (Buchko et al., 2021, Biomolecular NMR Assignments, 15:107-116, Dudás et al., 2021, Biomolecular NMR Assignments) (FIG. 9A). To improve the signals in HSQC maps, we decreased the temperature to slow down the exchange. The overlay of the HSQC spectra of Nsp9 obtained at 298 K and 278 K confirms that this was the case, for instance, with the increase of the intensities of T18, G17, G37, G61 and G63 cross-peaks, which should improve the confidence of the analysis (FIG. 12). The titrations were therefore carried at 278 K for 2NSP23 and 276 K for 2NSP90. Upon progressive addition of 2NSP23, an increasing number of amide cross-peaks of the protein disappeared (FIG. 10B), featuring the pattern expected for an intermediate exchange on the chemical shift scale, typically observed when 200-300 nM<KD<2-3 μM, where KD is the complex dissociation constant. By the end, at a protein/nanobody ratio of 1:2, only some 35% of the backbone amide cross-peaks survive (FIG. 10C). The same pattern was also observed with 2NSP90 with signal loss always pre-ceded by progressing intensity attenuation. Table 3 lists the HSQC signals of Nsp9 that disappear as a function of the concentration of added nanobody.

TABLE 3
Peak loss and attenuation in ¹⁵N-¹H HSQC spectra
of NSP9 titrations with nanobodies.
Peak loss

| Nanobody:NSP9 | | |
|---|---|---|
| ratio | 2NSP23^{(a)} | 2NSP90^{(a)} |
| 0.17:1 | — | — |
| 0.32:1 | — | D50, W53 |
| 0.43:1 | G17, F40, S46, K52, | A28, L69 |
| | T67, L69, Y89 | |
| 0.54:1 | M12, C14, T18, T19, C23, | M12, G17, T18, C23, |
| | A30, Y31, Y32, N33, T35, | A30, Y31, T35, F40, |
| | D50, W53, A54, F56, T64, | V41, L44, L45, K52, |
| | Y66, Y87, L88, I91, G93, | A54, F56, T67, E68, |
| | A108 | L88, Y89, G93, A108 |
| 0.63:1 | Q11, S13, A15, A22, N27, | Q11, S13, A15, T19, |
| | L29, T34, G37, L44, L45, | T21, A22, T24, N27, |
| | K58, D60, T62 I65, E68, | L29, Y32, N33, N33sc1, |
| | E70, C73, K86, F90, N95 | N33sc2 T34, G37, G38, |
| | | S46, W53sc, T62, G63, |
| | | T64, Y66, E70, R74, |
| | | I91, N95 |

Fast peak attenuation ^{(b)}

| 2NSP23 | 2NSP90 |
|---|---|
| C14, G17, T18, T19, A30, Y32, | S13, A30, Y31, N33, V41, A43, L51, |
| F40, S46, K52, A54, F56, T67, | W53, A54, F56, T67, L69, A108, R111 |
| L69, Y89, G93, A108 | |

^{(a)}Although quite similar in the relative interactions, the two nanobodies exhibit slight differences for the involved epitopes, with 2NSP23 addressing first the surface encompassing the fragments 50-53, 86-89, and 2NSP90 selecting only fragment 50-53.
^{(b)}The peaks with the steepest decrease in intensity were identified when the slope of their relative intensity attenuations was larger than the average attenuation slope increased by one standard deviation.

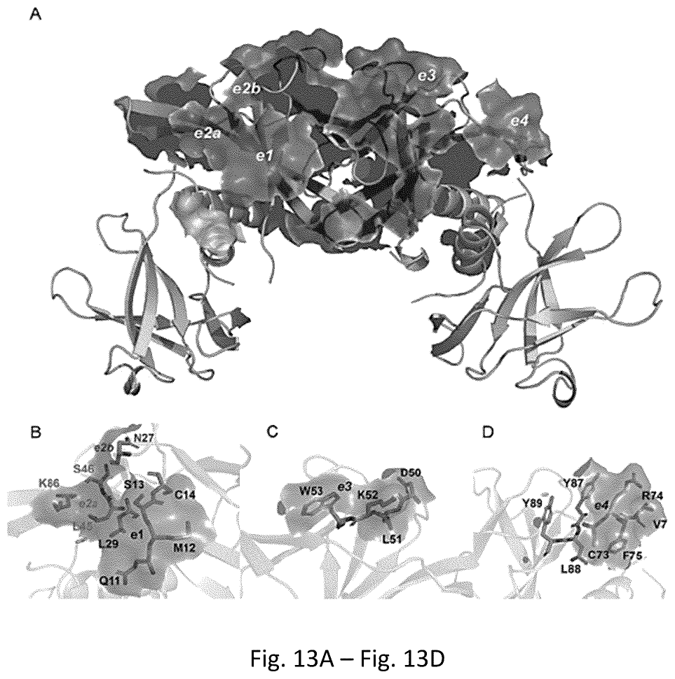

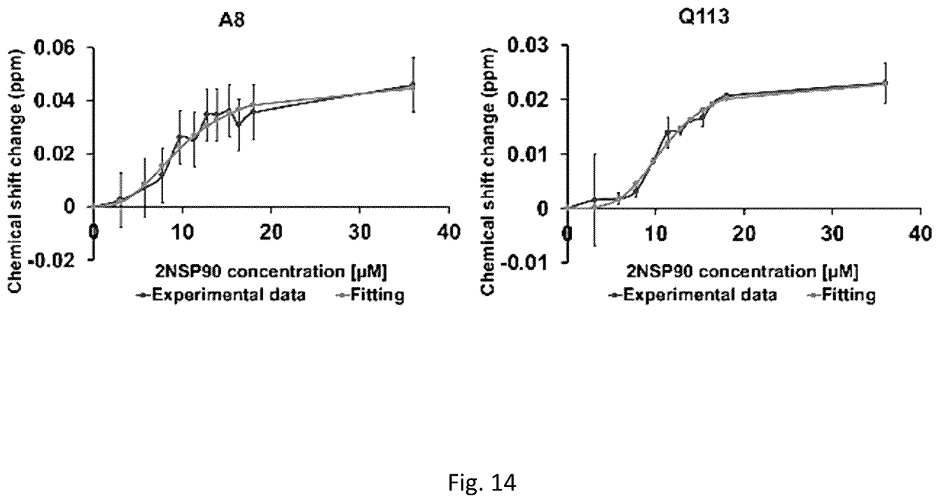

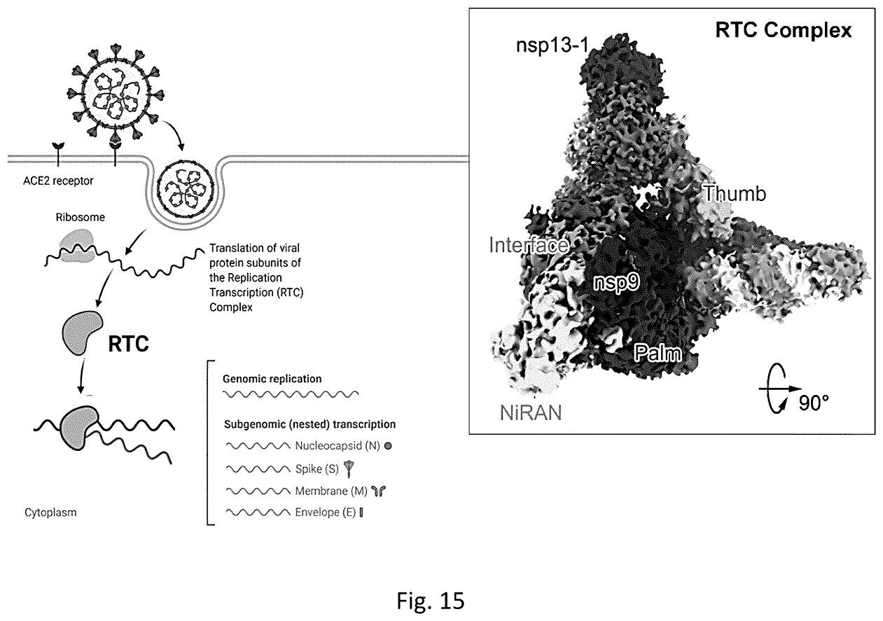

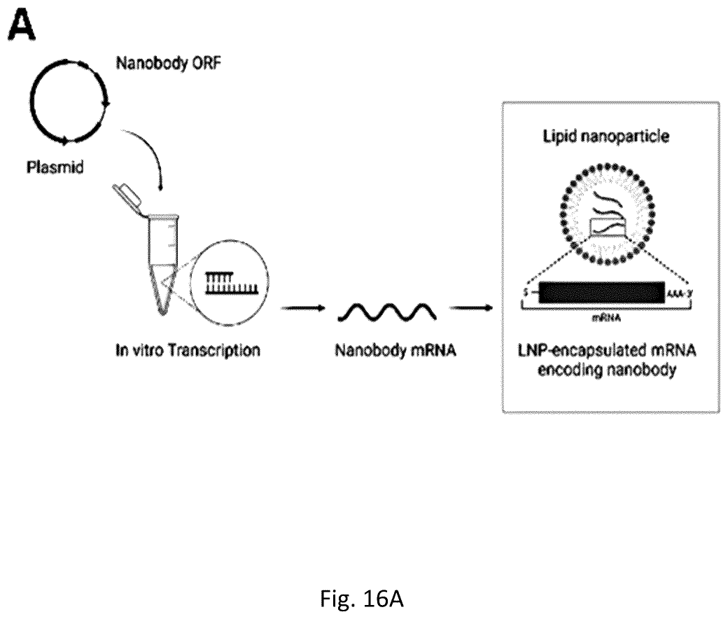

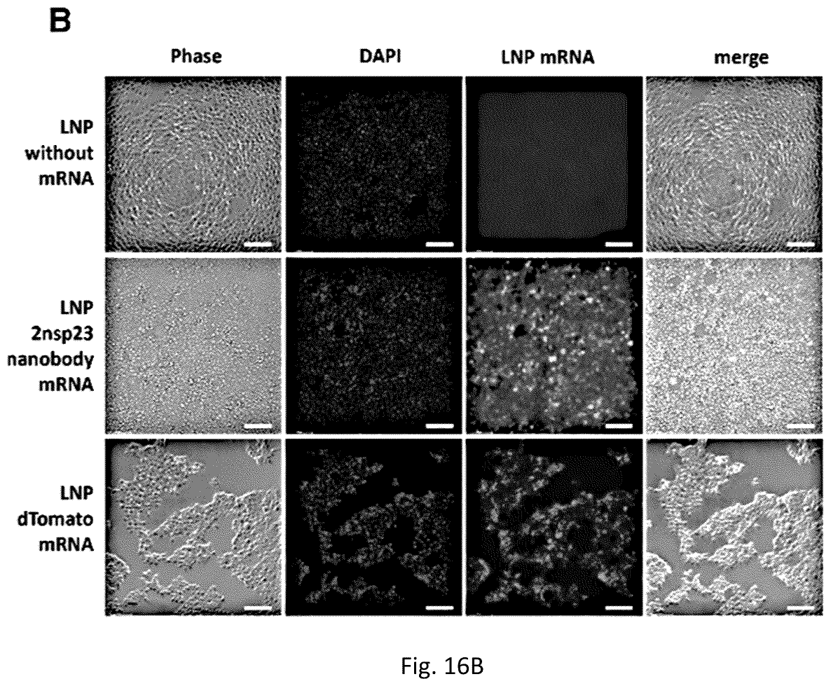

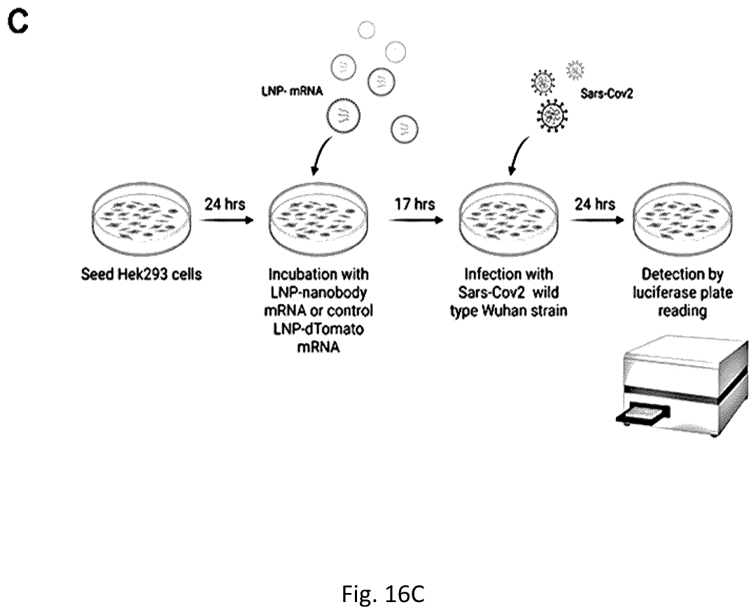

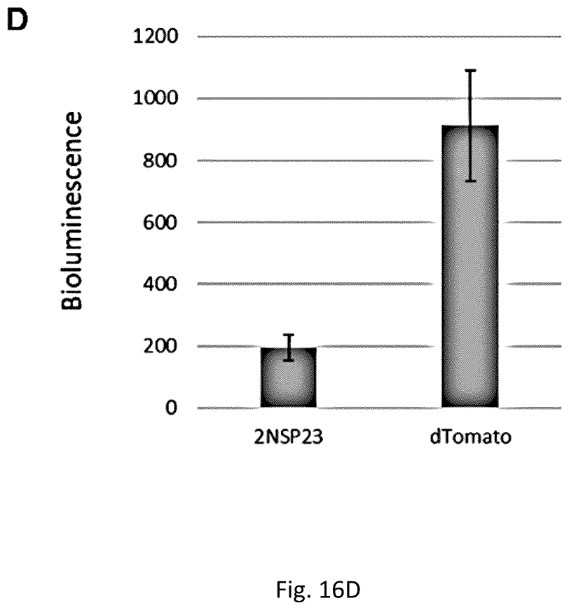

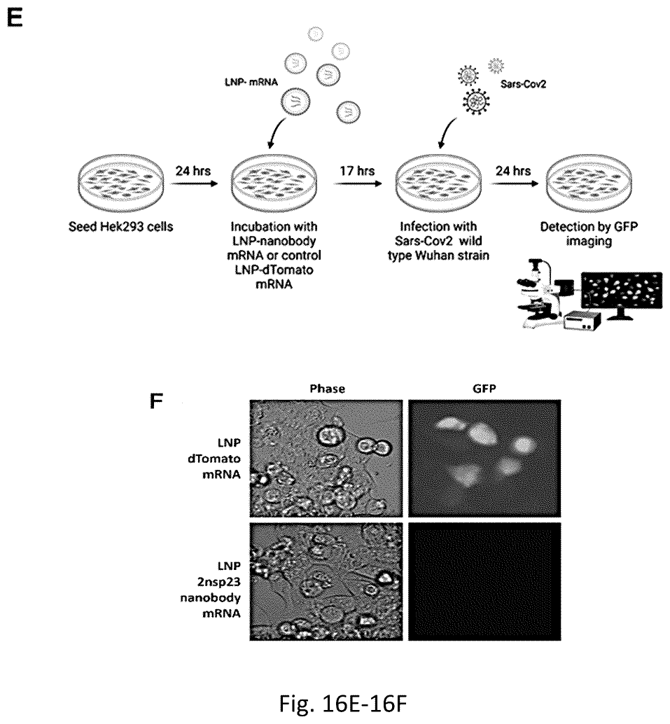

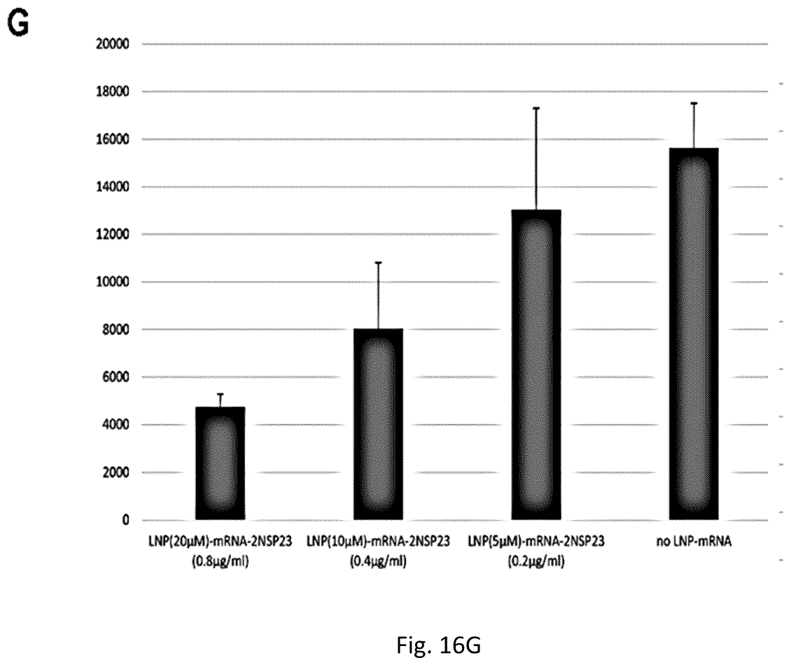

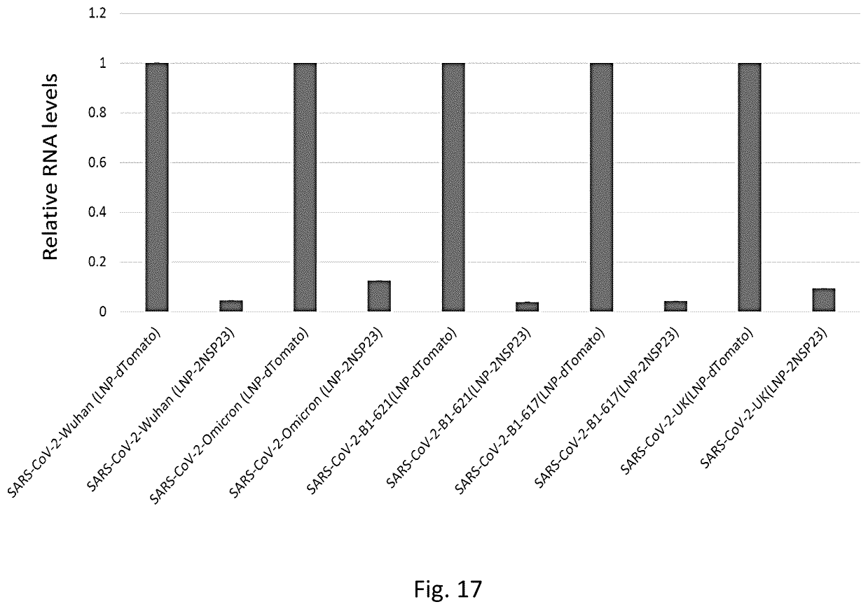

In addition to the cross-peak loss, the rate of intensity attenuation prior to loss is an indicative parameter and indicate that the two nanobodies are quite similar in the way they interact with Nsp9 (Table 3). In particular, the residues with high attenuation rates and the order of peak loss replicate the regions involved directly and indirectly in the tetramer assembly and the dimerization interface rearrangement, namely 67-69, 17-22, 37, 30-32, 44-46 and 108 (FIG. 13A), with extensions including adjacent segments or single residues. However, some fragments of Nsp9 undergo fast attenuation and/or subsequent signal loss that appear unrelated to the tetramerization interface, namely at positions 11-14, 27, 29, 50-53, 73-76, 86-89. These fragments cluster on two accessible surface regions flanking the tetramerization interface and should represent the epitopes of the Nsp9 tetramer for both nanobodies (FIG. 13). Each dimer of the Nsp9 tetramer contributes the two epitopes on opposite faces, hence the epitopes on the same face of the tetramer (FIG. 13A) are contributed by different dimers. The question arises on the number of nanobody monomers required to saturate the Nsp9 tetramer. A plausible stoichiometry for the Nsp9 tetramer could be four nanobody molecules. Evidence in favor of this stoichiometry comes from the fitting of the chemical shift variations observed for A8 and Q113 cross-peaks along Nsp9 titration with 2NSP90 (FIG. 10D), leading to statistically significant estimates of the number of nanobody-binding sites-between 3 and 4- and the half occupation constant of ˜10 μM (see FIG. 14). Besides the massive cross-peak loss representing the progressive propagation of the intermediate exchange regime with titrant saturation, we could also detect progressive chemical shift changes associated with titration (FIG. 10D). These involve mostly the N-terminal and C-terminal residue signals, namely A8, L9, R111 and Q113 and a couple of other locations (C73, V76). The pattern is compatible with the intermediate exchange regime observed for all the other residues of Nsp9 and may arise for intrinsically mobile molecular locations where the chemical shift is effectively averaged by the local dynamics, leading to a very small difference between the limiting chemical shift values and matching therefore local fast exchange regime.

### Example 2: Compositions that Block Activation of the SARS-CoV-2 Replication and Transcription Complex (RTC) and Methods of Use Thereof

A large number (136) of highly specific anti-Nsp9 nanobodies have been identified. Two of these nanobodies, 2NSP23 and 2NSP90, have been recombinantly expressed and purified, specifically recognize viral Nsp9 in saliva of Covid-19 positive patients. Using NMR spectroscopy, the epitopes of both nanobodies were mapped on Nsp9 and the data shows that they bind and tend to stabilize a tetrameric Nsp9 form (Esposito, G., et al., 2021. Adv. Biol. 5, e2101113; Hunashal, Y., et al. 2022. Anal Chem. 94, 10949-10958). Evidence of a composite binding pattern of the nanobodies to different Nsp9 forms is further supported by mass photometry data. Under the relative experimental conditions, 38/46/55 kDa complexes are detected when recombinant purified Nsp9 is mixed for 1 hr at RT with the nanobody at 1/1.5 ratio, whereas only a 42 kDa complex is detected at 1/0.5 ratio. With molecular masses of 12.5 kDa for Nsp9 and 15.8 kDa for the nanobodies, those figures could correspond to Nsp9: nanobody stoichiometries of 2:1, 1:2 and 2:2 expected to lead to complexes of 40.8, 44.1 and 56.6 kDa.

Altogether these studies are important because they suggest that anti-Nsp9 nanobodies might be specific diagnostic tools for rapid identification of Covid-19 patients and they also have a strong potential as viral inhibitors. Mechanistically, without being bound by theory, it is proposed that by binding to dimeric, tetrameric and, possibly, higher oligomeric forms of Nsp9, those nanobodies deplete the monomeric Nsp9 pool whose recruitment is required for the assembly of a functional RTC complex. Therefore, those nanobodies may serve as potential inhibitors of the RTC complex and viral replication as shown in FIG. 15, which depicts the SARS-CoV-2 life cycle. These observations have provided the foundations to file the present patent application.

The methods for preparation of the LNPs are now described

The lipid nanoparticle (LNP-mRNA) was prepared using NanoAssemblr® (Precision Nanosystems) microfluidic mixing technology under time invariant conditions. 2 ml of an aqueous solution containing the mRNA at a concentration of 174 μg/ml in aqueous 70 mM acetate buffer, pH 4.0, was mixed with 1 ml aqueous ethanolic lipid solution containing 12.5 mM lipids to form the nanoparticles. The flow rate ratio between the aqueous solution and the aqueous ethanolic lipid solution was 3:1, and the total flow rate was 12 ml/min.

The aqueous ethanolic lipid solution was prepared by dissolving C12-200 (Corden Pharma), DOPE (1,2-dioleoyl-sn-glycero-3-phosphoethanolamine), Cholesterol, DMG-PEG (PEGylated myristoyl diglyceride; Avanti catalog no. 880151P-1G) and DOTAP (1,2-di-(9Z-octadecenoyl)-3-trimethylammonium propane methylsulfate Avanti catalog no. 890890-200 mg) in a molar ratio of 29.8:13.6:39.5:2.1:15 in ethanol. This may be done by preparing separate 12.5 mM solutions of each lipid in the ethanol and mixing the solutions in the ratio given above to give the aqueous ethanolic lipid solution.

1.6 ml of the obtained LNP-mRNA product was immediately diluted with 64 ml PBS (1×) and concentrated to 1.5 ml at 2000×g for 30 minutes at 20° C. using Amicon® Ultra-15 centrifugal filtration tubes. Finally, the LNP sample was sterilized with 0.2 μm syringe filter and stored at 4° C. Size distribution of particles 70.7 nm, particle number 5.31E+11 and zeta potential 0.51 mV were measured with Zetasizer Ultra (Malvern Panalytical Ltd).

The experimental results are now described

### Anti-Nsp9 Nanobodies as Inhibitors of Sars-CoV-2 Replication

To explore whether the anti-Nsp9 nanobodies inhibit SARS-CoV-2, their ability to neutralize SARS-CoV-2 prior to cellular uptake was tested. For this purpose, a plaque assay was performed in the presence of anti-Nsp9 nanobodies. This quantitative assay is based on the number of plaques formed in cell culture upon infection with serial dilutions of a SARS-CoV-2 strain. Plaques form when a virus-infected cell lyses, leading to a subsequent cycle of infection and lysis of neighboring cells. So, by counting the number of virus plaques that are formed in the presence or absence of nanobodies, a direct measure of the nanobodies efficiency in neutralizing SARS-CoV-2 is obtained. In the assay, a confluent monolayer of HEK293-ACE2 cells was pre-incubated with recombinantly expressed and purified Nsp9-specific nanobodies 2NSP90, 2NSP23, 3NSP52, 3NSP78, 2INS27, 2INS45, 2INS64, 2INS69 or a control, unrelated nanobody (NB24), at serial dilutions (100, 50, 25, 12.5, 6.25, 3.1, 1.5 μg/ml). As positive control, a positive neutralizing serum from a patient obtained from our collaborating labs was used at 1:400 dilution. Cells were then infected with the wild-type Wuhan SARS-CoV-2 strain at virus inoculum 20-30 PFU (plaque forming units) and left for up to 72 hr at 37° C. Following infection, monolayers were covered with a solid overlay provided by carboxymethyl cellulose (CMC) and viral plaques were subsequently visualized by crystal violet. Complete inhibition (100%) was observed exclusively when cells were treated with the neutralizing serum as revealed by the lack of viral plaques which, as expected, were not detected in the absence of viral infection. In contrast, viral plaques were detected in the presence of the above anti-Nsp9 nanobodies and, likewise, in the presence of the unrelated nanobody NB24. It was concluded that Nsp9-specific nanobodies do not neutralize SARS-Cov-2 extracellularly, prior to cellular uptake, an observation compatible with the fact that Nsp9 and all other non-structural proteins involved in RTC assembly is not expressed in mature viral particles

To find out if, by targeting intracellular Nsp9, anti-Nsp9 nanobodies directly affect viral replication, 2NSP23 mRNA encapsulated into lipid nanoparticles (LNP)—referred to as LNP-mRNA-2NSP23—was designed and purchased from ISAR Bioscience, Munich, Germany, with the goal of having cells to uptake the mRNA and to intracellularly translate it into 2NSP23 nanobody (FIG. 16A). To prove cellular uptake, 105 HEK293-ACE2 cells were seeded in 24-wells plates and treated the next day with LNP (10 μM) and mRNA-2NSP23 (0.4 μg/ml) targeting SARS-CoV-2 Nsp9, or LNP (10 μM) and mRNA-NLP45 (0.4 μg/ml) expressing dTomato protein (also purchased from ISAR Bioscience, Munich, Germany). Twenty-four hours after LNP-mRNA treatment, cells were fixed and immunostained with llama's anti-VHH antibodies and imaged using a wide field microscope. Results from these experiments show that both nanobody 2NSP23 and dTomato protein are visualized intracellularly (FIG. 16B) which, in turn, indicates that both LNP-mRNA assemblies are taken up by cells and they are translated. Therefore, they may be used to test if they inhibit viral replication upon infection with SARS-CoV-2.

Based on the previous results, 10\hat{ }5 HEK293-ACE2 cells were seeded in 24-wells plates and treated the next day with LNP (10 μM) and mRNA-2NSP23 (0.4 μg/ml) expressing nanobody 2NSP23 to target SARS-CoV-2 Nsp9, or LNP (10 μM) and mRNA-NLP45 (0.4 μg/ml) expressing dTomato protein as a control. Twenty-four hours after LNP-mRNA treatment, cells were infected with SARS-CoV-2 Wuhan strain, engineered to express a nanoluciferase gene or a gene expressing the green fluorescent protein (GFP) (FIGS. 16C and 16E). Following 24 hr infection, the extent of viral replication was measured by monitoring bioluminescence with a plate reader or by imaging cells expressing GFP. Results from bioluminescence measurements from 3 independent experiments show that the amount of detected bioluminescence dropped by approximately 6 times when cells were pre-incubated with LNP-mRNA-2NSP23 in comparison with cells treated with LNP-mRNA-NLP45 expressing dTomato protein (FIG. 16D). GFP imaging of cells infected with SARS-CoV-2 expressing GFP shows no GFP expression upon LNP-mRNA-2NSP23 treatment (FIG. 16E) in contrast to cells treated with control LNP-mRNA-NLP45 expressing dTomato protein. As increasing amounts of 2NSP23, but not dTomato protein, appear to increasingly inhibit viral replication in a concentration dependent manner (FIG. 16F), taken altogether the results strongly suggest that 2NSP23 is a specific inhibitor of SARS-CoV-2 replication in cells and it has strong potential to be developed into an antiviral.

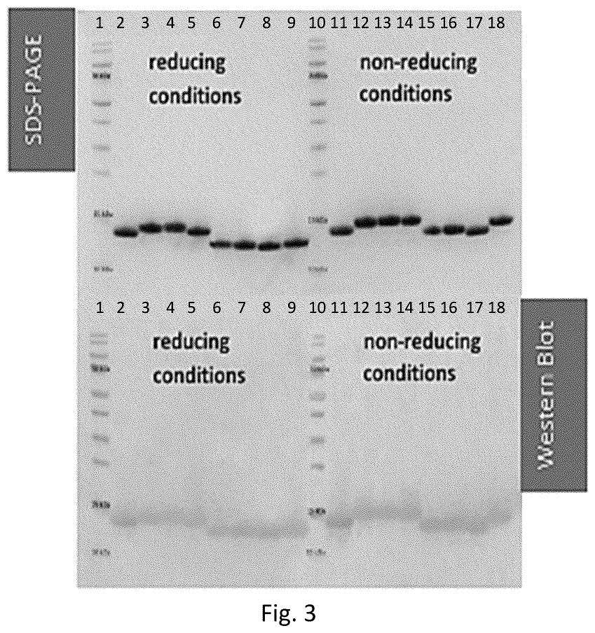

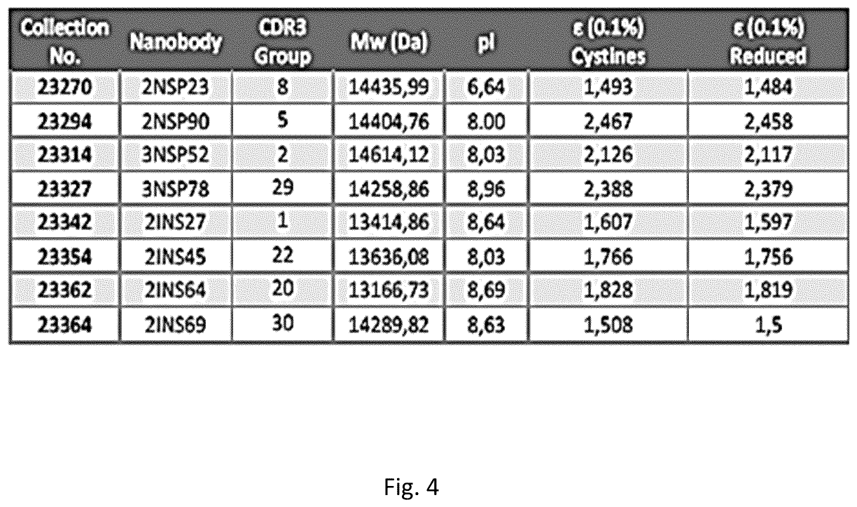

Nsp9 is highly conserved across the different SARS-CoV-2 strains so far originated during the pandemics. Previous data on geographical distribution of SARS-CoV-2 non-structural protein mutation report maximum frequencies for Nsp12, Nsp2 and Nsp3 (35.3, 26.4 and 11.7%, respectively), whereas Nsp9 mutations only account for 0.30% of non-structural protein sequence variability (Guruprasad K., 2021. ChemRxiv (2021), 10.33774/chemrxiv-2021-1f2zd-v2). In turn, non-structural protein mutations are not as frequent as the structural protein ones (including the Spike protein mutations) (Thakur S. et al, 2022. Front. Medicine, 9, 815389). Therefore, it was reasoned that 2NSP23 may inhibit replication of other SARS-CoV-2 variants by targeting Nsp9. To test this hypothesis, 10\hat{ }5 HEK293-ACE2 cells were seeded in 24-wells plates and treated the next day with LNP (10 μM) and mRNA-2NSP23 (0.4 μg/ml) expressing 2NSP23 to target SARS-CoV-2 Nsp9, or LNP (10 μM) and mRNA-NLP45 (0.4 μg/ml) expressing dTomato protein as a control. Twenty-four hours after LNP-mRNA treatment, cells were independently infected with several SARS-CoV2 strains, including Wuhan, alpha=UK=B1.1.7, Mu=B1.621, Delta=B1.617.x, and Omicron=B1.1.529, at M.O.I of 0.01. Following 24 hr infection, total RNAs from infected samples and a non-infected ones were prepared using Nucleospin RNA kit and quantified using a Nanodrop. Two ng of each sample, in triplicate, were used in a one-step qPCR to amplify and quantify the SARS-CoV-2 E gene using primer E_Sarbeco_F1: ACAGGTACGTTAATAGTTAATAGCGT (SEQ ID NO:129) and E_Sarbeco_R2: ATATTGCAGCAGTACGCACACA (SEQ ID NO:130) (Coupeau, D., et al., 2020. Methods Protoc. 3, 59). Relative viral RNAs were quantified according to the ΔΔCt standard method (Livak, K. J., Schmittgen, T. D., 2021. Method. Methods. 25, 402-8). The inhibition effect of LNP-mRNA-2NSP23 targeting Nsp9 on viral replication was determined relative to LNP-mRNA-NLP45 expressing dTomato protein. As can be seen in supplementary FIG. 3, results from qPCR analysis show that upon delivery of 2NSP23 mRNA and 2NSP23 intracellular expression, viral replication was significantly inhibited in all variants tested, in contrast to dTomato mRNA translation leading to intracellular dTomato protein, but no detectable inhibition (FIG. 17).

Based on the data, it was concluded that nanobody 2NSP23 mRNA can be efficiently delivered into human cells and once translated into the corresponding nanobody, it serves as a strong inhibitor of SARS-CoV-2 replication. Its efficacy appears conserved across within multiple SARS-CoV-2 strains including Wuhan, alpha-UK=B1.1.7, Mu=B1.621, Delta=B1.617.x, and Omicron=B1.1.529. Given the high degree of Nsp9 conservation across corona viruses (Guruprasad K., 2021. ChemRxiv (2021), doi: 10.33774/chemrxiv-2021-1f2zd-v2; Thakur S. et al, 2022. Front. Medicine, 9, 815389), nanobody 2NSP23 is likely to be a pan-inhibitor of coronaviruses replication. This conclusion is likely to be extended to the other 2NSP23-similar nanobodies that were isolated and tested in-vitro.

Sequences
SEQ ID NO: 1--2INS27 CDR1
SIFSSA
SEQ ID NO: 2--2INS27 CDR2
IGSSDT
SEQ ID NO: 3--2INS27 CDR3
KYGLGGFVY
SEQ ID NO: 4--2INS27 Full length Nanobody
QVQLQESGGGLVQAGGSLRLSCATSGSIFSSAVMGWYRQAPGNER
ELVALIGSSDTTDYSNSVKGRFTISRDNAKNTAYLRMNSLKPEDT
AVYYCTAVKYGLGGFVYWGQGTQVTVSS
SEQ ID NO: 5--2INS27 CDR1
AGCATCTTCAGTAGCGCT
SEQ ID NO: 6--2INS27 CDR2
ATTGGCAGTAGCGATACC
SEQ ID NO: 7--2INS27 CDR3
AAGTACGGGCTGGGGGGATTTGTCTAC
SEQ ID NO: 8--2INS27 Full length Nanobody
CAGGTGCAGCTGCAGGAGTCTGGGGGAGGCTTGGTGCAGGCTGGG
GGGTCTCTGAGACTCTCCTGTGCAACCTCTGGAAGCATCTTCAGT
AGCGCTGTCATGGGCTGGTACCGCCAGGCTCCAGGGAATGAGCGC
GAGTTGGTCGCACTCATTGGCAGTAGCGATACCACAGACTATTCA
AACTCTGTGAAGGGCCGATTCACCATCTCCAGAGACAACGCCAAG
AACACGGCCTATCTGCGCATGAATAGCCTGAAACCTGAGGACACA
GCCGTCTATTACTGTACTGCCGTCAAGTACGGGCTGGGGGGATTT
GTCTACTGGGGCCAGGGGACCCAGGTCACCGTCTCCTCA
SEQ ID NO: 9--3NSP52 CDR1
LTFDSY
SEQ ID NO: 10--3NSP52 CDR2
SIWSGDS
SEQ ID NO: 11--3NSP52 CDR3
ASFLHSANYHMRAKWGY
SEQ ID NO: 12--3NSP52 Full length Nanobody
QVQLQESGGGLVQPEGSLRLSCAASGLTFDSYAIGWFRQAPGKER
EFVAASIWSGDSGHYTGSVKGRFTISRDNAKNTVDLQMNSLKPED
TAVYYCAASASFLHSANYHMRAKWGYWGQGTQVTVSS
SEQ ID NO: 13--3NSP52 CDR1
CTCACCTTCGATAGCTAT
SEQ ID NO: 14--3NSP52 CDR2
AGTATCTGGAGTGGTGATAGC
SEQ ID NO: 15--3NSP52 CDR3
GCGTCTTTCTTGCACAGTGCTAATTACCACATGCGGGCAAAATGG
GGTTAC
SEQ ID NO: 16--3NSP52 Full length Nanobody
CAGGTGCAGCTGCAGGAGTCTGGGGGAGGATTGGTGCAGCCTGAG
GGCTCTCTGAGACTCTCCTGTGCAGCCTCTGGACTCACCTTCGAT
AGCTATGCCATCGGCTGGTTCCGCCAGGCTCCAGGGAAGGAGCGT
GAGTTTGTAGCAGCAAGTATCTGGAGTGGTGATAGCGGACACTAT
ACAGGTTCCGTGAAAGGCCGATTCACCATCTCCCGAGACAACGCC
AAGAACACGGTGGATCTGCAAATGAACAGCCTGAAACCCGAGGAC
ACGGCCGTTTATTACTGTGCAGCCAGCGCGTCTTTCTTGCACAGT
GCTAATTACCACATGCGGGCAAAATGGGGTTACTGGGGCCAGGGG
ACCCAGGTCACCGTCTCCTCA
SEQ ID NO: 17--2NSP90 CDR1
RTFSTY
SEQ ID NO: 18--2NSP90 CDR2
RWSGGT
SEQ ID NO: 19--2NSP90 CDR3
RGSGSYSPTYRWDY
SEQ ID NO: 20--2NSP90 Full length Nanobody
QVQLQESGGGLVQTGDSLRLSCAVSGRTFSTYSVGWFRQAPGKER
EFVALRWSGGTTYYADSVVGRFTVSRDNAKNTVYLEMNSLKPEDT
AVYYCAADRGSGSYSPTYRWDYWGQGTQVTVSS
SEQ ID NO: 21--2NSP90 CDR1
CGCACCTTCAGTACCTAT
SEQ ID NO: 22--2NSP90 CDR2
AGGTGGAGTGGTGGTACC
SEQ ID NO: 23--2NSP90 CDR3
CGGGGTAGTGGTAGTTACTCCCCGACATATCGCTGGGACTAT
SEQ ID NO: 24--2NSP90 Full length Nanobody
CAGGTGCAGCTGCAGGAGTCTGGGGGAGGGTTGGTGCAAACTGGG
GACTCTCTGAGACTCTCCTGTGCAGTGTCTGGACGCACCTTCAGT
ACCTATTCCGTGGGCTGGTTCCGCCAGGCTCCAGGAAAGGAGCGT
GAGTTTGTAGCGCTTAGGTGGAGTGGTGGTACCACATACTATGCA
GACTCCGTGGTGGGCCGGTTCACCGTCTCCAGAGACAATGCCAAG
AACACGGTGTATCTGGAAATGAACAGCCTGAAACCTGAGGACACG
GCCGTTTATTACTGTGCAGCAGATCGGGGTAGTGGTAGTTACTCC
CCGACATATCGCTGGGACTATTGGGGCCAGGGGACCCAGGTCACC
GTCTCCTCA
SEQ ID NO: 25--2NSP23 CDR1
LAFSMY
SEQ ID NO: 26--2NSP23 CDR2
IISSGDS
SEQ ID NO: 27--2NSP23 CDR3
KFRYYFSTSPGDFDS
SEQ ID NO: 28--2NSP23 Full length Nanobody
QVQLQESGGGLVQPGGSLRLSCAASGLAFSMYTMGWFRQAPGKER
EFVAMIISSGDSTDYADSVKGRFTISRDNGKNTVYLQMDSLKPED
TAVYYCAAPKFRYYFSTSPGDFDSWGQGTQVTVSS
SEQ ID NO: 29--2NSP23 CDR1
CTCGCCTTTAGTATGTAT
SEQ ID NO: 30--2NSP23 CDR2
ATTATTTCAAGTGGTGAT
SEQ ID NO: 31--2NSP23 CDR3
AAGTTTCGTTACTACTTTAGCACCTCTCCAGGTGATTTTGATTCC
SEQ ID NO: 32--2NSP23 Full length Nanobody
CAGGTGCAGCTGCAGGAGTCTGGAGGAGGATTGGTACAGCCTGGG
GGCTCTCTGAGACTCTCCTGTGCAGCCTCTGGACTCGCCTTTAGT
ATGTATACCATGGGCTGGTTCCGCCAGGCTCCAGGGAAGGAGCGT
GAGTTTGTAGCAATGATTATTTCAAGTGGTGATAGCACCGACTAC
GCAGACTCCGTGAAGGGCCGATTCACCATCTCCAGGGACAACGGC
AAGAACACGGTGTATCTGCAAATGGACAGCCTGAAACCTGAGGAC
ACGGCCGTTTATTACTGTGCAGCCCCAAAGTTTCGTTACTACTTT
AGCACCTCTCCAGGTGATTTTGATTCCTGGGGCCAGGGGACCCAG
GTCACCGTCTCCTCA
SEQ ID NO: 33--2INS64 CDR1
SILSIN
SEQ ID NO: 34--2INS64 CDR2
ITSGGS
SEQ ID NO: 35--2INS64 CDR3
TGWG PLD
SEQ ID NO: 36--2INS64 Full length Nanobody
QVQLQESGGGLVQVGGSLRLSCAASGSILSINAMGWYRQAPGKQR
ELVAAITSGGSTNYADSVKGRFTISRDNAKNMLYLQMNSLKPEDT
AVYYCHVVTGWGPLDWGQGTQVTVSS
SEQ ID NO: 37--2INS64 CDR1
AGCATCCTCAGTATCAAT
SEQ ID NO: 38--2INS64 CDR2
ATTACTAGTGGTGGTAGC
SEQ ID NO: 39--2INS64 CDR3
ACGGGTTGGGGTCCCCTAGAC
SEQ ID NO: 40--2INS64 Full length Nanobody
CAGGTGCAGCTGCAGGAGTCTGGGGGAGGCTTGGTGCAGGTTGGG
GGGTCTCTACGACTCTCCTGTGCAGCCTCTGGAAGCATCCTCAGT
ATCAATGCCATGGGCTGGTACCGCCAGGCTCCAGGGAAGCAGCGC
GAGTTGGTCGCAGCTATTACTAGTGGTGGTAGCACAAACTATGCA
GACTCCGTGAAGGGCCGATTCACCATCTCCAGAGACAACGCCAAG
AACATGCTATATCTGCAAATGAACAGCCTGAAACCTGAGGACACA
GCCGTCTATTACTGCCACGTCGTTACGGGTTGGGGTCCCCTAGAC
TGGGGCCAGGGGACCCAGGTCACCGTCTCCTCA
SEQ ID NO: 41--2INS45 CDR1
FTLSSN
SEQ ID NO: 42--2INS45 CDR2
ITSGLS
SEQ ID NO: 43--2INS45 CDR3
RGWGPPRDY
SEQ ID NO: 44--2INS45 Full length Nanobody
QVQLQESGGGLVQAGGSLRLSCAASGFTLSSNDMGWYRQAPGKQR
ELVATITSGLSTNYADSVKGRFTISRDNAKNTVFLQMNSLKIEDT
AVYYCEVERGWGPPRDYWGHGTQVTVSS
SEQ ID NO: 45--2INS45 CDR1
TTTACCTTAAGTAGCAAT
SEQ ID NO: 46--2INS45 CDR2
ATTACTAGTGGTCTGAGC
SEQ ID NO: 47--2INS45 CDR3
AGGGGTTGGGGACCGCCGAGGGACTAC
SEQ ID NO: 48--2INS45 Full length Nanobody
CAGGTGCAGCTGCAGGAGTCTGGGGGAGGCTTGGTGCAAGCTGGG
GGGTCTCTGAGACTCTCCTGCGCAGCCTCTGGATTTACCTTAAGT
AGCAATGACATGGGCTGGTACCGCCAGGCTCCAGGGAAGCAGCGC
GAATTGGTCGCAACTATTACTAGTGGTCTGAGCACAAACTATGCA
GACTCCGTGAAGGGCCGATTCACCATCTCCAGAGACAACGCCAAG
AACACGGTGTTTCTGCAAATGAATAGCCTGAAAATTGAGGACACA
GCCGTCTATTACTGTGAGGTAGAGAGGGGTTGGGGACCGCCGAGG
GACTACTGGGGCCACGGGACCCAGGTCACCGTCTCCTCA
SEQ ID NO: 49--3NSP78 CDR1
RAFSSY
SEQ ID NO: 50--3NSP78 CDR2
IHWTGAA
SEQ ID NO: 51--3NSP78 CDR3
PSGSYWPPKRYDY
SEQ ID NO: 52--3NSP78 Full length Nanobody
QVQLQESGGGLVQAGGSLRLSCAASGRAFSSYVMGWFRQAPGKER
EFVAAIHWTGAATVYVDSVKGRFAISRHNAKNTVYLEMNSLKPED
TAVYYCAADPSGSYWPPKRYDYWGQGTQVTVSS
SEQ ID NO: 53--3NSP78 CDR1
CGCGCCTTCAGTAGTTAT
SEQ ID NO: 54--3NSP78 CDR2
ATTCATTGGACTGGTGCTGCT
SEQ ID NO: 55--3NSP78 CDR3
CCTAGCGGTAGTTACTGGCCCCCCAAGAGGTATGACTAC
SEQ ID NO: 56--3NSP78 Full length Nanobody
CAGGTGCAGCTGCAGGAGTCTGGAGGAGGATTGGTGCAGGCTGGG
GGCTCTCTGAGACTCTCCTGTGCAGCCTCTGGACGCGCCTTCAGT
AGTTATGTTATGGGCTGGTTTCGCCAGGCTCCAGGAAAGGAGCGT
GAGTTTGTAGCAGCTATTCATTGGACTGGTGCTGCTACAGTCTAT
GTAGACTCCGTGAAGGGTCGATTCGCCATCTCCAGACACAACGCC
AAGAACACGGTGTATCTGGAAATGAACAGCCTGAAACCTGAGGAC
ACGGCCGTTTATTACTGTGCAGCAGACCCTAGCGGTAGTTACTGG
CCCCCCAAGAGGTATGACTACTGGGGCCAGGGGACCCAGGTCACC
GTCTCCTCA
SEQ ID NO: 57--2INS69 CDR1
RTFSSV
SEQ ID NO: 58--2INS69 CDR2
ISGSTGSV
SEQ ID NO: 59--2INS69 CDR3
FTGTFNYQGLYDY
SEQ ID NO: 60--2INS69 Full length Nanobody
QVQLQESGGGLVRAGDSLRLSCAVSGRTFSSVAMGWFRQAPGKER
EFVAFISGSTGSVTYYADSVKGRFAISRDNAKNTVYLQMNSLKPE
DTAVYDCAAKFTGTFNYQGLYDYWGQGTQVTVSS
SEQ ID NO: 61--2INS69 CDR1
CGCACCTTCAGTAGCGTT
SEQ ID NO: 62--2INS69 CDR2
ATTAGCGGGAGTACTGGTAGTGTT
SEQ ID NO: 63--2INS69 CDR3
TTTACTGGTACTTTCAACTACCAAGGTCTATATGACTAC
SEQ ID NO: 64--2INS69 Full length Nanobody
CAGGTGCAGCTGCAGGAGTCTGGGGGAGGATTGGTCCGCGCTGGG
GACTCTCTGAGACTCTCCTGTGCAGTCTCTGGACGCACCTTCAGT
AGCGTTGCCATGGGCTGGTTCCGCCAGGCTCCAGGGAAGGAGCGT
GAATTTGTAGCGTTTATTAGCGGGAGTACTGGTAGTGTTACATAC
TATGCAGACTCCGTGAAGGGCCGATTCGCCATCTCCAGAGACAAC
GCCAAGAACACGGTGTATCTACAAATGAACAGCCTGAAGCCTGAG
GACACGGCCGTTTATGACTGTGCAGCTAAGTTTACTGGTACTTTC
AACTACCAAGGTCTATATGACTACTGGGGCCAGGGGACCCAGGTC
ACCGTCTCCTCA
SEQ ID NO: 65--3NSP56 CDR1
RTFSRY
SEQ ID NO: 66--3NSP56 CDR2
INWSGTS
SEQ ID NO: 67--3NSP56 CDR3
LVRNYRLGWGDGVYDH
SEQ ID NO: 68--3NSP56 Full length Nanobody
QVQLQESGGGLVQAGGSLRLSCAASGRTFSRYAMGWFRQAPGKER
ESVAAINWSGTSIFYANSVEGRFTISRDNAKNTVYLQLNSLKPED
TAVYYCAADLVRNYRLGWGDGVYDHYGQGTQVTVSS
SEQ ID NO: 69--3NSP56 CDR1
CGCACCTTCAGTAGGTAT
SEQ ID NO: 70--3NSP56 CDR2
ATTAATTGGAGTGGTACTAGC
SEQ ID NO: 71--3NSP56 CDR3
TTGGTTCGGAACTACAGATTGGGCTGGGGAGATGGCGTCTATGAC
CAC
SEQ ID NO: 72--3NSP56 Full length Nanobody
CAGGTGCAGCTGCAGGAGTCTGGGGGAGGATTGGTGCAGGCTGGG
GGCTCTCTGAGACTCTCCTGTGCAGCCTCTGGACGCACCTTCAGT
AGGTATGCTATGGGCTGGTTCCGCCAGGCTCCAGGGAAGGAGCGT
GAGTCTGTAGCAGCTATTAATTGGAGTGGTACTAGCATATTCTAT
GCAAACTCCGTGGAGGGCCGATTCACCATCTCCAGAGACAACGCC
AAGAACACGGTGTATCTGCAACTGAACAGCCTGAAACCTGAGGAC
ACGGCCGTTTATTACTGTGCAGCAGACTTGGTTCGGAACTACAGA
TTGGGCTGGGGAGATGGCGTCTATGACCACTACGGCCAGGGGACC
CAGGTCACCGTCTCCTCA
SEQ ID NO: 73--3NSP29 CDR1
RTFSRY
SEQ ID NO: 74--3NSP29 CDR2
INWSGTS
SEQ ID NO: 75--3NSP29 CDR3
LNGMPY
SEQ ID NO: 76--3NSP29 Full length Nanobody
QVQLQESGGGLVQAGGSLRLSCAASGRTFSRYAMGWFRQAPGKER
ESVAAINWSGTSIFYANSVEGRFTISRDNAKNTVYLQLNSLKPED
TAVYYCGVVLNGMPYWGRGTQVTVSS
SEQ ID NO: 77--3NSP29 CDR1
CGCACCTTCAGTAGGTAT
SEQ ID NO: 78--3NSP29 CDR2
ATTAATTGGAGTGGTACTAGC
SEQ ID NO: 79--3NSP29 CDR3
CTGAACGGCATGCCCTAC
SEQ ID NO: 80--3NSP29 Full length Nanobody
CAGGTGCAGCTGCAGGAGTCTGGGGGAGGGCTGGTGCAGGCTGGG
GGCTCTCTGAGACTCTCCTGTGCAGCCTCTGGACGCACCTTCAGT
AGGTATGCTATGGGCTGGTTCCGCCAGGCTCCAGGGAAGGAGCGT
GAGTCTGTAGCAGCTATTAATTGGAGTGGTACTAGCATATTCTAT
GCAAACTCCGTGGAGGGCCGATTCACCATCTCCAGAGACAACGCC
AAGAACACGGTGTATCTGCAACTGAACAGCCTGAAACCTGAGGAC
ACGGCCGTTTATTACTGTGGAGTTGTCCTGAACGGCATGCCCTAC
TGGGGCAGAGGGACCCAGGTCACCGTCTCCTCA
SEQ ID NO: 81--2INS61 CDR1
SIFSIN
SEQ ID NO: 82--2INS61 CDR2
ITSGGS
SEQ ID NO: 83--2INS61 CDR3
EGWG PPVGY
SEQ ID NO: 84--2INS61 Full length Nanobody
QVQLQESGGGLVQAGGSLRLSCAASGSIFSINTMGWYRQAPGKQR
ELVAAITSGGSTNYADSVKGRFTISRDNAKNTVHLQMNSLKPEDT
AVYYCNVVEGWGPPVGYWGQGTQVTVSS
SEQ ID NO: 85--2INS61 CDR1
AGCATCTTCAGTATCAAT
SEQ ID NO: 86--2INS61 CDR2
ATTACTAGTGGTGGTAGC
SEQ ID NO: 87--2INS61 CDR3
GAGGGTTGGGGACCACCTGTAGGCTAC
SEQ ID NO: 88--2INS61 Full length Nanobody
CAGGTGCAGCTGCAGGAGTCTGGGGGAGGCTTGGTGCAGGCTGGG
GGGTCTCTGAGACTCTCCTGTGCAGCCTCTGGAAGCATCTTCAGT
ATCAATACCATGGGCTGGTACCGCCAGGCTCCAGGGAAGCAGCGC
GAGTTGGTCGCAGCTATTACTAGTGGTGGTAGCACAAACTATGCA
GACTCCGTGAAGGGCCGATTCACCATCTCCAGAGACAACGCCAAA
AACACGGTACATCTGCAAATGAACAGCCTGAAACCTGAGGACACA
GCCGTCTATTACTGTAATGTAGTTGAGGGTTGGGGACCACCTGTA
GGCTACTGGGGCCAGGGGACCCAGGTCACCGTCTCCTCA
SEQ ID NO: 89--3INS39 CDR1
IMFGIN
SEQ ID NO: 90--3INS39 CDR2
ITSSGS
SEQ ID NO: 91--3INS39 CDR3
AGWGPPPGY
SEQ ID NO: 92--3INS39 Full length Nanobody
QVQLQESGGGLVQAGGSLRVSCEVSGIMFGINTMGWYRQAPGKQR
ELVAHITSSGSTNYADSVKGRFTISRDNAKKTVYLQMNSLKPEDT
AVYYCNVVAGWGPPPGYWGQGTQVTVSS
SEQ ID NO: 93--3INS39 CDR1
ATCATGTTTGGAATTAAT
SEQ ID NO: 94--3INS39 CDR2
ATCACTAGTAGTGGTAGC
SEQ ID NO: 95--3INS39 CDR3
GCAGGTTGGGGTCCGCCACCCGGCTAC
SEQ ID NO: 96--3INS39 Full length Nanobody
CAGGTGCAGCTGCAGGAGTCTGGAGGAGGCTTGGTGCAGGCTGGG
GGGTCTCTGAGAGTCTCCTGTGAAGTCTCTGGAATCATGTTTGGA
ATTAATACCATGGGCTGGTACCGCCAGGCTCCAGGGAAGCAGCGC
GAATTGGTCGCACACATCACTAGTAGTGGTAGCACAAATTATGCA
GACTCCGTGAAGGGCCGATTCACCATCTCCAGAGACAACGCCAAG
AAAACGGTGTATCTGCAAATGAACAGTCTGAAACCTGAGGACACA
GCCGTGTACTACTGCAATGTAGTTGCAGGTTGGGGTCCGCCACCC
GGCTACTGGGGCCAGGGGACCCAGGTCACCGTCTCCTCA
2INS27 with His tag
SEQ ID NO: 105
QVQLQESGGGLVQAGGSLRLSCATSGSIFSSAVMGWYRQAPGNER
ELVALIGSSDTTDYSNSVKGRFTISRDNAKNTAYLRMNSLKPEDT
AVYYCTAVKYGLGGFVYWGQGTQVTVSSAAAYPYDVPDYGSHHHH
HH
SEQ ID NO: 106
CAGGTGCAGCTGCAGGAGTCTGGGGGAGGCTTGGTGCAGGCTGGG
GGGTCTCTGAGACTCTCCTGTGCAACCTCTGGAAGCATCTTCAGT
AGCGCTGTCATGGGCTGGTACCGCCAGGCTCCAGGGAATGAGCGC
GAGTTGGTCGCACTCATTGGCAGTAGCGATACCACAGACTATTCA
AACTCTGTGAAGGGCCGATTCACCATCTCCAGAGACAACGCCAAG
AACACGGCCTATCTGCGCATGAATAGCCTGAAACCTGAGGACACA
GCCGTCTATTACTGTACTGCCGTCAAGTACGGGCTGGGGGGATTT
GTCTACTGGGGCCAGGGGACCCAGGTCACCGTCTCCTCAGCGGCC
GCATACCCGTACGACGTTCCGGACTACGGTTCCCACCACCATCAC
CATCAC
3NSP52 with His Tag
SEQ ID NO: 107
QVQLQESGGGLVQPEGSLRLSCAASGLTFDSYAIGWFRQAPGKER
EFVAASIWSGDSGHYTGSVKGRFTISRDNAKNTVDLQMNSLKPED
TAVYYCAASASFLHSANYHMRAKWGYWGQGTQVTVSSAAAYPYDV
PDYGSHHHHHH
SEQ ID NO: 108
CAGGTGCAGCTGCAGGAGTCTGGGGGAGGATTGGTGCAGCCTGAG
GGCTCTCTGAGACTCTCCTGTGCAGCCTCTGGACTCACCTTCGAT
AGCTATGCCATCGGCTGGTTCCGCCAGGCTCCAGGGAAGGAGCGT
GAGTTTGTAGCAGCAAGTATCTGGAGTGGTGATAGCGGACACTAT
ACAGGTTCCGTGAAAGGCCGATTCACCATCTCCCGAGACAACGCC
AAGAACACGGTGGATCTGCAAATGAACAGCCTGAAACCCGAGGAC
ACGGCCGTTTATTACTGTGCAGCCAGCGCGTCTTTCTTGCACAGT
GCTAATTACCACATGCGGGCAAAATGGGGTTACTGGGGCCAGGGG
ACCCAGGTCACCGTCTCCTCAGCGGCCGCATACCCGTACGACGTT
CCGGACTACGGTTCCCACCACCATCACCATCAC
2NSP90 with His Tag
SEQ ID NO: 109
QVQLQESGGGLVQTGDSLRLSCAVSGRTFSTYSVGWFRQAPGKER
EFVALRWSGGTTYYADSVVGRFTVSRDNAKNTVYLEMNSLKPEDT
AVYYCAADRGSGSYSPTYRWDYWGQGTQVTVSSAAAYPYDVPDYG
SHHHHHH
SEQ ID NO: 110
CAGGTGCAGCTGCAGGAGTCTGGGGGAGGGTTGGTGCAAACTGGG
GACTCTCTGAGACTCTCCTGTGCAGTGTCTGGACGCACCTTCAGT
ACCTATTCCGTGGGCTGGTTCCGCCAGGCTCCAGGAAAGGAGCGT
GAGTTTGTAGCGCTTAGGTGGAGTGGTGGTACCACATACTATGCA
GACTCCGTGGTGGGCCGGTTCACCGTCTCCAGAGACAATGCCAAG
AACACGGTGTATCTGGAAATGAACAGCCTGAAACCTGAGGACACG
GCCGTTTATTACTGTGCAGCAGATCGGGGTAGTGGTAGTTACTCC
CCGACATATCGCTGGGACTATTGGGGCCAGGGGACCCAGGTCACC
GTCTCCTCAGCGGCCGCATACCCGTACGACGTTCCGGACTACGGT
TCCCACCACCATCACCATCAC
2NSP23 with His Tag
SEQ ID NO: 111
QVQLQESGGGLVQPGGSLRLSCAASGLAFSMYTMGWFRQAPGKER
EFVAMIISSGDSTDYADSVKGRFTISRDNGKNTVYLQMDSLKPED
TAVYYCAAPKFRYYFSTSPGDFDSWGQGTQVTVSSAAAYPYDVPD
YGSHHHHHH
SEQ ID NO: 112
CAGGTGCAGCTGCAGGAGTCTGGAGGAGGATTGGTACAGCCTGGG
GGCTCTCTGAGACTCTCCTGTGCAGCCTCTGGACTCGCCTTTAGT
ATGTATACCATGGGCTGGTTCCGCCAGGCTCCAGGGAAGGAGCGT
GAGTTTGTAGCAATGATTATTTCAAGTGGTGATAGCACCGACTAC
GCAGACTCCGTGAAGGGCCGATTCACCATCTCCAGGGACAACGGC
AAGAACACGGTGTATCTGCAAATGGACAGCCTGAAACCTGAGGAC
ACGGCCGTTTATTACTGTGCAGCCCCAAAGTTTCGTTACTACTTT
AGCACCTCTCCAGGTGATTTTGATTCCTGGGGCCAGGGGACCCAG
GTCACCGTCTCCTCAGCGGCCGCATACCCGTACGACGTTCCGGAC
TACGGTTCCCACCACCATCACCATCAC
2INS64 with His Tag
SEQ ID NO: 113
QVQLQESGGGLVQVGGSLRLSCAASGSILSINAMGWYRQAPGKQR
ELVAAITSGGSTNYADSVKGRFTISRDNAKNMLYLQMNSLKPEDT
AVYYCHVVTGWGPLDWGQGTQVTVSSAAAYPYDVPDYGSHHHHHH
SEQ ID NO: 114
CAGGTGCAGCTGCAGGAGTCTGGGGGAGGCTTGGTGCAGGTTGGG
GGGTCTCTACGACTCTCCTGTGCAGCCTCTGGAAGCATCCTCAGT
ATCAATGCCATGGGCTGGTACCGCCAGGCTCCAGGGAAGCAGCGC
GAGTTGGTCGCAGCTATTACTAGTGGTGGTAGCACAAACTATGCA
GACTCCGTGAAGGGCCGATTCACCATCTCCAGAGACAACGCCAAG
AACATGCTATATCTGCAAATGAACAGCCTGAAACCTGAGGACACA
GCCGTCTATTACTGCCACGTCGTTACGGGTTGGGGTCCCCTAGAC
TGGGGCCAGGGGACCCAGGTCACCGTCTCCTCAGCGGCCGCATAC
CCGTACGACGTTCCGGACTACGGTTCCCACCACCATCACCATCAC
2INS45 with His Tag
SEQ ID NO: 115
QVQLQESGGGLVQAGGSLRLSCAASGFTLSSNDMGWYRQAPGKQR
ELVATITSGLSTNYADSVKGRFTISRDNAKNTVFLQMNSLKIEDT
AVYYCEVERGWGPPRDYWGHGTQVTVSSAAAYPYDVPDYGSHHHH
HH
SEQ ID NO: 116
CAGGTGCAGCTGCAGGAGTCTGGGGGAGGCTTGGTGCAAGCTGGG
GGGTCTCTGAGACTCTCCTGCGCAGCCTCTGGATTTACCTTAAGT
AGCAATGACATGGGCTGGTACCGCCAGGCTCCAGGGAAGCAGCGC
GAATTGGTCGCAACTATTACTAGTGGTCTGAGCACAAACTATGCA
GACTCCGTGAAGGGCCGATTCACCATCTCCAGAGACAACGCCAAG
AACACGGTGTTTCTGCAAATGAATAGCCTGAAAATTGAGGACACA
GCCGTCTATTACTGTGAGGTAGAGAGGGGTTGGGGACCGCCGAGG
GACTACTGGGGCCACGGGACCCAGGTCACCGTCTCCTCAGCGGCC
GCATACCCGTACGACGTTCCGGACTACGGTTCCCACCACCATCAC
CATCAC
3NSP78 with His Tag
SEQ ID NO: 117:
QVQLQESGGGLVQAGGSLRLSCAASGRAFSSYVMGWFRQAPGKER
EFVAAIHWTGAATVYVDSVKGRFAISRHNAKNTVYLEMNSLKPED
TAVYYCAADPSGSYWPPKRYDYWGQGTQVTVSSAAAYPYDVPDYG
SHHHHHH
SEQ ID NO: 118
CAGGTGCAGCTGCAGGAGTCTGGAGGAGGATTGGTGCAGGCTGGG
GGCTCTCTGAGACTCTCCTGTGCAGCCTCTGGACGCGCCTTCAGT
AGTTATGTTATGGGCTGGTTTCGCCAGGCTCCAGGAAAGGAGCGT
GAGTTTGTAGCAGCTATTCATTGGACTGGTGCTGCTACAGTCTAT
GTAGACTCCGTGAAGGGTCGATTCGCCATCTCCAGACACAACGCC
AAGAACACGGTGTATCTGGAAATGAACAGCCTGAAACCTGAGGAC
ACGGCCGTTTATTACTGTGCAGCAGACCCTAGCGGTAGTTACTGG
CCCCCCAAGAGGTATGACTACTGGGGCCAGGGGACCCAGGTCACC
GTCTCCTCAGCGGCCGCATACCCGTACGACGTTCCGGACTACGGT
TCCCACCACCATCACCATCAC
2INS69 with His Tag
SEQ ID NO: 119
QVQLQESGGGLVRAGDSLRLSCAVSGRTFSSVAMGWFRQAPGKER
EFVAFISGSTGSVTYYADSVKGRFAISRDNAKNTVYLQMNSLKPE
DTAVYDCAAKFTGTFNYQGLYDYWGQGTQVTVSSAAAYPYDVPDY
GSHHHHHH
SEQ ID NO: 120
CAGGTGCAGCTGCAGGAGTCTGGGGGAGGATTGGTCCGCGCTGGG
GACTCTCTGAGACTCTCCTGTGCAGTCTCTGGACGCACCTTCAGT
AGCGTTGCCATGGGCTGGTTCCGCCAGGCTCCAGGGAAGGAGCGT
GAATTTGTAGCGTTTATTAGCGGGAGTACTGGTAGTGTTACATAC
TATGCAGACTCCGTGAAGGGCCGATTCGCCATCTCCAGAGACAAC
GCCAAGAACACGGTGTATCTACAAATGAACAGCCTGAAGCCTGAG
GACACGGCCGTTTATGACTGTGCAGCTAAGTTTACTGGTACTTTC
AACTACCAAGGTCTATATGACTACTGGGGCCAGGGGACCCAGGTC
ACCGTCTCCTCAGCGGCCGCATACCCGTACGACGTTCCGGACTAC
GGTTCCCACCACCATCACCATCAC
3NSP56 with His Tag
SEQ ID NO: 121
QVQLQESGGGLVQAGGSLRLSCAASGRTFSRYAMGWFRQAPGKER
ESVAAINWSGTSIFYANSVEGRFTISRDNAKNTVYLQLNSLKPED
TAVYYCAADLVRNYRLGWGDGVYDHYGQGTQVTVSSAAAYPYDVP
DYGSHHHHHH
SEQ ID NO: 122
CAGGTGCAGCTGCAGGAGTCTGGGGGAGGATTGGTGCAGGCTGGG
GGCTCTCTGAGACTCTCCTGTGCAGCCTCTGGACGCACCTTCAGT
AGGTATGCTATGGGCTGGTTCCGCCAGGCTCCAGGGAAGGAGCGT
GAGTCTGTAGCAGCTATTAATTGGAGTGGTACTAGCATATTCTAT
GCAAACTCCGTGGAGGGCCGATTCACCATCTCCAGAGACAACGCC
AAGAACACGGTGTATCTGCAACTGAACAGCCTGAAACCTGAGGAC
ACGGCCGTTTATTACTGTGCAGCAGACTTGGTTCGGAACTACAGA
TTGGGCTGGGGAGATGGCGTCTATGACCACTACGGCCAGGGGACC
CAGGTCACCGTCTCCTCAGCGGCCGCATACCCGTACGACGTTCCG
GACTACGGTTCCCACCACCATCACCATCAC
3NSP29 with His Tag
SEQ ID NO: 123
QVQLQESGGGLVQAGGSLRLSCAASGRTFSRYAMGWFRQAPGKER
ESVAAINWSGTSIFYANSVEGRFTISRDNAKNTVYLQLNSLKPED
TAVYYCGVVLNGMPYWGRGTQVTVSSAAAYPYDVPDYGSHHHHHH
SEQ ID NO: 124
CAGGTGCAGCTGCAGGAGTCTGGGGGAGGGCTGGTGCAGGCTGGG
GGCTCTCTGAGACTCTCCTGTGCAGCCTCTGGACGCACCTTCAGT
AGGTATGCTATGGGCTGGTTCCGCCAGGCTCCAGGGAAGGAGCGT
GAGTCTGTAGCAGCTATTAATTGGAGTGGTACTAGCATATTCTAT
GCAAACTCCGTGGAGGGCCGATTCACCATCTCCAGAGACAACGCC
AAGAACACGGTGTATCTGCAACTGAACAGCCTGAAACCTGAGGAC
ACGGCCGTTTATTACTGTGGAGTTGTCCTGAACGGCATGCCCTAC
TGGGGCAGAGGGACCCAGGTCACCGTCTCCTCAGCGGCCGCATAC
CCGTACGACGTTCCGGACTACGGTTCCCACCACCATCACCATCAC
2INS61 with His Tag
SEQ ID NO: 125
QVQLQESGGGLVQAGGSLRLSCAASGSIFSINTMGWYRQAPGKQR
ELVAAITSGGSTNYADSVKGRFTISRDNAKNTVHLQMNSLKPEDT
AVYYCNVVEGWGPPVGYWGQGTQVTVSSAAAYPYDVPDYGSHHHH
HH
SEQ ID NO: 126
CAGGTGCAGCTGCAGGAGTCTGGGGGAGGCTTGGTGCAGGCTGGG
GGGTCTCTGAGACTCTCCTGTGCAGCCTCTGGAAGCATCTTCAGT
ATCAATACCATGGGCTGGTACCGCCAGGCTCCAGGGAAGCAGCGC
GAGTTGGTCGCAGCTATTACTAGTGGTGGTAGCACAAACTATGCA
GACTCCGTGAAGGGCCGATTCACCATCTCCAGAGACAACGCCAAA
AACACGGTACATCTGCAAATGAACAGCCTGAAACCTGAGGACACA
GCCGTCTATTACTGTAATGTAGTTGAGGGTTGGGGACCACCTGTA
GGCTACTGGGGCCAGGGGACCCAGGTCACCGTCTCCTCAGCGGCC
GCATACCCGTACGACGTTCCGGACTACGGTTCCCACCACCATCAC
CATCAC
3INS39 with His Tag
SEQ ID NO: 127
QVQLQESGGGLVQAGGSLRVSCEVSGIMFGINTMGWYRQAPGKQR
ELVAHITSSGSTNYADSVKGRFTISRDNAKKTVYLQMNSLKPEDT
AVYYCNVVAGWGPPPGYWGQGTQVTVSSAAAYPYDVPDYGSHHHH
HH
SEQ ID NO: 128
CAGGTGCAGCTGCAGGAGTCTGGAGGAGGCTTGGTGCAGGCTGGG
GGGTCTCTGAGAGTCTCCTGTGAAGTCTCTGGAATCATGTTTGGA
ATTAATACCATGGGCTGGTACCGCCAGGCTCCAGGGAAGCAGCGC
GAATTGGTCGCACACATCACTAGTAGTGGTAGCACAAATTATGCA
GACTCCGTGAAGGGCCGATTCACCATCTCCAGAGACAACGCCAAG
AAAACGGTGTATCTGCAAATGAACAGTCTGAAACCTGAGGACACA
GCCGTGTACTACTGCAATGTAGTTGCAGGTTGGGGTCCGCCACCC
GGCTACTGGGGCCAGGGGACCCAGGTCACCGTCTCCTCAGCGGCC
GCATACCCGTACGACGTTCCGGACTACGGTTCCCACCACCATCAC
CATCAC

The disclosures of each and every patent, patent application, and publication cited herein are hereby incorporated herein by reference in their entirety.

While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. An isolated antibody or antibody fragment that specifically binds to SARS-CoV-2 non-structural protein, wherein the target SARS-CoV-2 non-structural protein is selected from the group consisting of Nsp7, Nsp8, Nsp9, Nsp12 and Nsp13.

2. The antibody or antibody fragment of claim 1, wherein the antibody or antibody fragment is selected from the group consisting of a monoclonal antibody, a polyclonal antibody, a single chain antibody, an immunoconjugate, a glycoengineered antibody, and a bispecific antibody or other multi-specific antibody.

3. The antibody or antibody fragment of claim 2, wherein the antibody is a single chain antibody, and further wherein the single chain antibody is a nanobody.

4. The antibody or antibody fragment of claim 3, wherein the nanobody is specific for binding to Nsp9.

5. The antibody or antibody fragment of claim 4, wherein the antibody comprises at least one selected from the group consisting of:
a) at least one CDR sequence selected from the group consisting of a CDR1 of SEQ ID NO:1, a CDR2 of SEQ ID NO:2 and a CDR3 of SEQ ID NO:3;
b) at least one CDR sequence selected from the group consisting of a CDR1 of SEQ ID NO:9, a CDR2 of SEQ ID NO: 10 and a CDR3 of SEQ ID NO:11;
c) at least one CDR sequence selected from the group consisting of a CDR1 of SEQ ID NO:17, a CDR2 of SEQ ID NO: 18 and a CDR3 of SEQ ID NO: 19;
d) at least one CDR sequence selected from the group consisting of a CDR1 of SEQ ID NO:25, a CDR2 of SEQ ID NO:26 and a CDR3 of SEQ ID NO:27;
e) at least one CDR sequence selected from the group consisting of a CDR1 of SEQ ID NO:33, a CDR2 of SEQ ID NO:34 and a CDR3 of SEQ ID NO:35;
f) at least one CDR sequence selected from the group consisting of a CDR1 of SEQ ID NO:41, a CDR2 of SEQ ID NO:42 and a CDR3 of SEQ ID NO:43;
g) at least one CDR sequence selected from the group consisting of a CDR1 of SEQ ID NO:49, a CDR2 of SEQ ID NO:50 and a CDR3 of SEQ ID NO:51; and
h) at least one CDR sequence selected from the group consisting of a CDR1 of SEQ ID NO:57, a CDR2 of SEQ ID NO:58 and a CDR3 of SEQ ID NO:59.

6. The antibody or antibody fragment of claim 5, wherein the antibody comprises at least one selected from the group consisting of:
a) all three CDR sequence selected from the group consisting of a CDR1 of SEQ ID NO:1, a CDR2 of SEQ ID NO:2 and a CDR3 of SEQ ID NO:3;
b) all three CDR sequence selected from the group consisting of a CDR1 of SEQ ID NO:9, a CDR2 of SEQ ID NO: 10 and a CDR3 of SEQ ID NO: 11;
c) all three CDR sequence selected from the group consisting of a CDR1 of SEQ ID NO: 17, a CDR2 of SEQ ID NO: 18 and a CDR3 of SEQ ID NO:19;
d) all three CDR sequence selected from the group consisting of a CDR1 of SEQ ID NO:25, a CDR2 of SEQ ID NO:26 and a CDR3 of SEQ ID NO:27;
e) all three CDR sequence selected from the group consisting of a CDR1 of SEQ ID NO:33, a CDR2 of SEQ ID NO:34 and a CDR3 of SEQ ID NO:35;
f) all three CDR sequence selected from the group consisting of a CDR1 of SEQ ID NO:41, a CDR2 of SEQ ID NO:42 and a CDR3 of SEQ ID NO:43;
g) all three CDR sequence selected from the group consisting of a CDR1 of SEQ ID NO:49, a CDR2 of SEQ ID NO:50 and a CDR3 of SEQ ID NO:51; and
h) all three CDR sequence selected from the group consisting of a CDR1 of SEQ ID NO:57, a CDR2 of SEQ ID NO:58 and a CDR3 of SEQ ID NO:59.

7. The antibody or antibody fragment of claim 6, wherein the antibody comprises an amino acid sequence selected from the group consisting of SEQ ID NO:4, SEQ ID NO: 12, SEQ ID NO:20, SEQ ID NO:28, SEQ ID NO:36, SEQ ID NO:44, SEQ ID NO:52, and SEQ ID NO:60.

8. A composition comprising an antibody or antibody fragment of claim 1.

9. The composition of claim 8, comprising a lipid nanoparticle (LNP) comprising the antibody or antibody fragment.

10. A nucleic acid molecule comprising a nucleotide sequence encoding an antibody or antibody fragment of claim 1.

11. The nucleic acid molecule of claim 10, comprising nucleotide monomer units selected from RNA, DNA and chemically modified nucleotide monomer units.

12. The nucleic acid molecule of claim 10, comprising RNA nucleotide monomer units and further comprises one or more nucleotide monomer units selected from DNA and chemically modified nucleotide monomer units.

13. The nucleic acid molecule of claim 10, which is an RNA molecule.

14. The nucleic acid molecule of claim 10, comprising a 5′-CAP and/or a poly-A tail.

15. The nucleic acid molecule of claim 10, comprising at least one selected from the group consisting of:
a) at least one nucleotide sequence selected from the group consisting of SEQ ID NO:5, SEQ ID NO:6 and SEQ ID NO:7, encoding CDR1, CDR2 and CDR3 respectively;
b) at least one nucleotide sequence selected from the group consisting of SEQ ID NO:13, SEQ ID NO:14 and SEQ ID NO:15, encoding CDR1, CDR2 and CDR3 respectively;
c) at least one nucleotide sequence selected from the group consisting of SEQ ID NO:21, SEQ ID NO:22 and SEQ ID NO:23, encoding CDR1, CDR2 and CDR3 respectively;
d) at least one nucleotide sequence selected from the group consisting of SEQ ID NO:29, SEQ ID NO:30 and SEQ ID NO:31, encoding CDR1, CDR2 and CDR3 respectively;
e) at least one nucleotide sequence selected from the group consisting of SEQ ID NO:37, SEQ ID NO:38 and SEQ ID NO:39, encoding CDR1, CDR2 and CDR3 respectively;
f) at least one nucleotide sequence selected from the group consisting of SEQ ID NO:45, SEQ ID NO:46 and SEQ ID NO:47, encoding CDR1, CDR2 and CDR3 respectively;
g) at least one nucleotide sequence selected from the group consisting of SEQ ID NO:53, SEQ ID NO:54 and SEQ ID NO:55, encoding CDR1, CDR2 and CDR3 respectively; and
h) at least one nucleotide sequence selected from the group consisting of SEQ ID NO:61, SEQ ID NO:62 and SEQ ID NO:63, encoding CDR1, CDR2 and CDR3 respectively.

16. The nucleic acid molecule of claim 15, comprising at least one selected from the group consisting of:
a) a nucleotide sequence comprising each of SEQ ID NO:5, SEQ ID NO:6 and SEQ ID NO:7, encoding CDR1, CDR2 and CDR3 respectively;
b) a nucleotide sequence comprising each of SEQ ID NO:13, SEQ ID NO:14 and SEQ ID NO:15, encoding CDR1, CDR2 and CDR3 respectively;
c) a nucleotide sequence comprising each of SEQ ID NO:21, SEQ ID NO:22 and SEQ ID NO:23, encoding CDR1, CDR2 and CDR3 respectively;
d) a nucleotide sequence comprising each of SEQ ID NO:29, SEQ ID NO:30 and SEQ ID NO:31, encoding CDR1, CDR2 and CDR3 respectively;
e) a nucleotide sequence comprising each of SEQ ID NO:37, SEQ ID NO:38 and SEQ ID NO:39, encoding CDR1, CDR2 and CDR3 respectively;
f) a nucleotide sequence comprising each of SEQ ID NO:45, SEQ ID NO:46 and SEQ ID NO:47, encoding CDR1, CDR2 and CDR3 respectively;
g) a nucleotide sequence comprising each of SEQ ID NO:53, SEQ ID NO:54 and SEQ ID NO:55, encoding CDR1, CDR2 and CDR3 respectively; and
h) a nucleotide sequence comprising each of SEQ ID NO:61, SEQ ID NO:62 and SEQ ID NO:63, encoding CDR1, CDR2 and CDR3 respectively.

17. The nucleic acid molecule of claim 16, comprising at least one selected from the group consisting of: SEQ ID NO:8, SEQ ID NO:16, SEQ ID NO:24, SEQ ID NO:32, SEQ ID NO:40, SEQ ID NO:48, SEQ ID NO:56, and SEQ ID NO:64.

18. A composition comprising a nucleic acid molecule of claim 10.

19. The composition of claim 18, comprising a lipid nanoparticle (LNP) comprising the nucleic acid molecule.

20. The composition of claim 19, comprising wherein the LNP comprises one or more lipids.

21. The composition of claim 19, wherein the LNP comprises at least one ionizable cationic lipid.

22. The composition of claim 21, wherein the ionizable cationic lipid is C12-200.

23. The composition of claim 19, wherein the LNP comprises at least one neutral amphoteric or zwitterionic lipid.

24. The composition of claim 23, wherein the neutral amphoteric or zwitterionic lipid comprises DOPE.

25. The composition of claim 19, wherein the LNP comprises cholesterol.

26. The composition of claim 19, wherein the LNP comprises at least one non-ionic lipid.

27. The composition of claim 26, wherein the at least one non-ionic lipid comprises at least one PEGylated non-ionic lipid.

28. The composition of claim 27, wherein the PEGylated non-ionic lipid comprises DMG-PEG.

29. The composition of claim 19, wherein the LNP comprises at least one quaternary ammonium cationic lipid.

30. The composition of claim 29, wherein the at least one quaternary ammonium cationic lipid comprises DOTAP.

31. The composition of claim 19, wherein the LNP comprises two or more lipids selected from the group consisting of an ionizable cationic lipid, a neutral amphoteric or zwitterionic lipid, a non-ionic lipid, cholesterol, and a quaternary ammonium cationic lipid.

32. The composition of claim 19, wherein the LNP comprises an ionizable cationic lipid, a neutral amphoteric or zwitterionic lipid, a non-ionic lipid, cholesterol, and a quaternary ammonium cationic lipid.

33. The composition of claim 32, wherein the LNP comprises C12-200, DOPE, cholesterol, DMG-PEG and DOTAP.

34. The composition of claim 18, wherein the nucleic acid molecule is an RNA molecule encoding the antibody or antibody fragment.

35. An expression vector comprising a nucleic acid molecule of claim 10.

36. A host cell comprising a nucleic acid molecule of claim 10.

37. A method of preventing SARS-CoV-2 viral replication in a subject in need thereof, the method comprising the step of administering a composition comprising the antibody or antibody fragment of claim 1; or a nucleic acid molecule encoding the antibody or antibody fragment to a subject in need thereof.

38. A method of treating or preventing a disease or disorder associated with SARS-CoV-2 infection in a subject in need thereof, the method comprising the step of administering a composition comprising the antibody or antibody fragment of claim 1; or a nucleic acid molecule encoding the antibody or antibody fragment to a subject in need thereof.

39. The method of claim 38, wherein the disease associated with SARS-CoV-2 infection comprises COVID-19.

40. A method of detecting a SARS-CoV-2 Nsp in a sample, the method comprising:
a) contacting the sample with a composition comprising the antibody or antibody fragment of claim 1; or a nucleic acid molecule encoding the antibody or antibody fragment, and
b) detecting binding of the antibody or antibody fragment to the target SARS-CoV-2 Nsp.

41. A method of diagnosing SARS-CoV-2 infection in a subject in need thereof, the method comprising the steps of:
a. contacting a biological sample of the subject with a composition comprising the antibody or antibody fragment of claim 1; or a nucleic acid molecule encoding the antibody or antibody fragment,
b. determining the presence of the SARS-CoV-2 Nsp in the biological sample of the subject, and
c. diagnosing the subject with a SARS-CoV-2 infection when SARS-CoV-2 Nsp is detected the in the biological sample of the subject.

42. The method of claim 41, comprising the further step of administering a treatment to the subject that was diagnosed as having SARS-CoV-2 infection.
